(19) United States
(12) Patent Application Publication
(10) Pub. No.: US 2013/0011393 A1
(43) Pub. Date: Jan. 10, 2013

(54) BAD PATHWAY GENE SIGNATURE

(76) Inventors: Johnathan Mark Lancaster, Tampa, FL (US); Dung-Tsa Chen, Tampa, FL (US); Yin Xiong, Lutz, FL (US)

(21) Appl. No.: 13/519,544

(22) PCT Filed: Jan. 12, 2011
(86) PCT No.: PCT/US2011/021013

§ 371 Date: Sep. 24, 2012

Related U.S. Application Data
(60) Provisional application No. 61/294,168, filed on Jan. 12, 2010.

(51) Int. Cl. C40B 30/04; C40B 40/06; G06F 19/24; A61K 39/395

(52) U.S. Cl. 424/133.1; 506/9; 506/16; 702/19

(57) ABSTRACT

The invention provides materials and methods for prognosing cancer, and predicting an individual's responsiveness to cancer treatments, methods of treating cancer, and materials and methods for obtaining BAD pathway gene expression profiles useful in carrying out the methods of the invention.

### CROSS-REFERENCE TO RELATED APPLICATION

The present application claims the benefit of U.S. Provisional Application Ser. No. 61/294,168, filed Jan. 12, 2010, which is hereby incorporated by reference herein in its entirety, including any figures, tables, nucleic acid sequences, amino acid sequences, and drawings.

### GOVERNMENT SUPPORT

This invention was made with Government support under Grant No. W81XWH-08-2-0101 awarded by the Department of Defense (ARMY/MRMC). The Government has certain rights in the invention.

### BACKGROUND OF THE INVENTION

BAD (BCL-2 associated death promoter) is a member of the BCL-2 family of proteins, which are characterized by the presence of up to 4 BCL-2-homology domains (Danial et al, “Cell death: critical control points” Cell, 2004, 116:205-219). This family includes inhibitors and promoters of apoptosis, such that cell survival versus death is determined by the relative ratio of pro-apoptotic (e.g., BCL-Xs, BAD, Bax, Bak) and anti-apoptotic (e.g., Bcl-2, Bcl-xL, MCL-1, A1, BAG-1) family members (Danial et al. “Cell death: critical control points” Cell, 2004, 116:205-219; Dejean et al. “Oligomeric Bax is a component of the putative cytochrome c release channel MAC, mitochondrial apoptosis-induced channel” Mol Biol Cell, 2005, 16:2424-2432; Desagher et al. “Bid-induced conformational change of Bax is responsible for mitochondrial cytochrome c release during apoptosis” J Cell Biol, 1999, 144:891-901; Kuwana et al. “Bid, Bax, and lipids cooperate to form supramolecular openings in the outer mitochondrial membrane” Cell, 2002, 111:331-342). BAD selectively hetero-dimerizes with Bcl-xL and Bcl-2 but not with Bax, Bcl-xs, Mcl-1, A1, or itself. When BAD dimerizes with Bcl-xL, Bax is displaced, mitochondrial membrane permeability increases, and apoptosis is induced (Yang et al. “Bad, a heterodimeric partner for Bcl-XL and Bcl-2, displaces Bax and promotes cell death” Cell, 1995, 80:285-291). However, BAD function is regulated by phosphorylation (including serine-112, -136, and -155). When phosphorylated, BAD is unable to heterodimerize with Bcl-2 or Bcl-xL, freeing Bcl-xL to dimerize and functionally sequestrate Bax, such that it is no longer free to induce apoptosis (Yang et al. “Bad, a heterodimeric partner for Bcl-XL and Bcl-2, displaces Bax and promotes cell death” Cell, 1995, 80:285-291). Thus, the phosphorylation status of BAD determines whether Bax is displaced from Bcl-xL to drive cell death. BAD is thought to be phorphorylated at serine-136 by protein kinase B (PKB/Akt) (del Peso et al. “Interleukin-3-induced phosphorylation of BAD through the protein kinase Akt” Science, 1997, 278:687-689). In contrast, serine-112 is phosphorylated by mitogen-activated protein kinase-activated protein kinase-1 (MAPKAP-K1, also called RSK) and PKA. Serine-155, at the center of the BAD BH3 domain, is phosphorylated preferentially by PKA, which also inhibits Bcl-xL binding (Lizcano et al. “Regulation of BAD by cAMP-dependent protein kinase is mediated via phosphorylation of a novel site, Ser155” Biochem J, 2000, 349:547-557; Tan et al. “BAD Ser-155 phosphorylation regulates BAD/Bcl-XL interaction and cell survival” J Biol Chem, 2000, 275:25865-25869; Zhou et al. “Growth factors inactivate the cell death promoter BAD by phosphorylation of its BH3 domain on Ser155” J Biol Chem, 2000, 275:25046-25051). Conversely, the activity of a series of phosphatases, including PP1, PP2A, and PPM1 (PP2C/PPM1A), as well as calcineurin, has been shown to have pro-apoptotic effects via de-phosphorylation of BAD (Klumpp et al. “Protein phosphatase type 2C dephosphorylates BAD” Neurochem. Int, 2003, 42:555-560).

### BRIEF SUMMARY OF THE INVENTION

The invention provides biomarkers based on the gene expression of members of the BCL-2 associated death promoter (BAD) pathway, which can discriminate between patients with longer versus shorter survival from many human cancers. The present invention relates to the use of genes from the BAD pathway as prognostic biomarkers for various human cancers including but not limited to ovarian cancer, brain cancer, and breast cancer.

The invention provides compositions and methods for predicting the development and progression of cancer, and predicting an individual's responsiveness to cancer treatments, methods of treating cancer, and methods of obtaining BAD pathway gene expression profiles useful in carrying out the methods of the invention.

The BAD pathway is a critical driver of cellular apoptosis control and a key component of ovarian cancer (OVCA) chemo-sensitivity. A BAD pathway gene expression signature was developed which identified 53 genes in the BAD apoptosis pathway. A pathway score was developed to represent an overall gene expression level for the 53 BAD pathway genes, and subsets thereof. The influence of the BAD pathway expression signature (also referred to herein as a “BAD pathway signature”, “BAD pathway score” or simply “pathway score”) on cancer patient survival (overall survival or relapse-free survival) for various datasets was evaluated. The BAD pathway expression signature has clinical utility as a prognostic biomarker for various types of cancer.

The present invention provides methods and materials (e.g., kits, arrays, and other compositions of matter) for preparing a gene expression profile indicative of cancer prognosis, or cancer chemo-resistance/chemo-sensitivity. In one aspect, the present invention is a method for preparing a gene expression profile indicative of cancer prognosis, comprising: obtaining a biological sample, and determining the level of expression for a plurality of genes of the BCL2 antagonist of cell death (BAD) pathway, thereby preparing the gene expression profile. The sample may be a biological sample from a subject or a cell line, for example.

In some embodiments, the prognosis is with respect to at least one factor selected from the group consisting of overall survival, disease- or relapse-free survival, and rate of progression of tumor. In some embodiments, the prognosis is with respect to disease development or progression (e.g., metastasis, transition, tumor size progression, progression from chemo-sensitivity to chemo-resistance). In some embodiments, the prognosis is with respect to survival, and the cancer is selected from among ovarian cancer, breast cancer, colon cancer, and brain cancer. In some embodiments, the prognosis is with respect to development and progression of cancer, and the cancer is selected from among breast cancer and endometrial cancer. By predicting the subject's prognosis, this aspect of the invention thereby provides information to guide individualized cancer treatment.

Preferably, the plurality of genes of the BAD pathway used for the gene expression profile comprises a plurality of genes listed in Table 1.

In some embodiments, the plurality of BAD pathway genes is 53 BAD pathway genes, or a subset thereof. In some embodiments, the plurality of BAD pathway genes is 43 BAD pathway genes or 47 BAD pathway genes. In some embodiments, the 53 BAD pathway genes are those represented by U133Plus of Table 1. In some embodiments, the 47 BAD pathway genes are those represented by U133A of Table 1.

In some embodiments, the plurality of BAD pathway genes comprises or consists of the following 53 BAD pathway genes: MAP2K2, RAF1, HRAS, SCH1, EGFR, IRS1, PIK3CA, PIK3CB, PIK3CD, RPS6KA1, RPS6KA2, RPS6KA3, BAD, BCL2L1, BAX, AKT1, AKT2, AKT3, GNB1, GNB2, GNB3, GNG4, GNB5, GNG10, GNG11, GNG12, GNG13, GNG2, GNG3, GNG4, GNG5, GNG7, GNG8, GNGT1, GNGT2, PPM1A, PPM1B, PPM1D, PPM1F, PPM1G, PPM1L, PPM2C, PPTC7, PTPN11, GNAS, PRKAR1A, PRKAR1B, PRKAR2A, PRKAR2B, CDC2, PRKACA, PRKACB, and PRKACG.

In some embodiments, the plurality of BAD pathway genes comprises or consists of the following 47 BAD pathway genes: MAP2K2, RAF1, HRAS, SCH1, EGFR, IRS1, PIK3CA, PIK3CB, PIK3CD, RPS6KA1, RPS6KA2, RPS6KA3, BAD, BCL2L1, BAX, AKT1, AKT2, AKT3, GNB1, GNB2, GNB3, GNB5, GNG10///LOC552891, GNG11, GNG12, GNG13, GNG3, GNG4, GNG5, GNG7, GNGT1, PPM1A, PPM1B, PPM1D, PPM1F, PPM1G, PPM2C, PTPN11, GNAS, PRKAR1A, PRKAR1B, PRKAR2A, PRKAR2B, CDC2, PRKACA, PRKACB, and PRKACG.

Preferably, the gene expression profile is expressed as a pathway score (also referred to herein as a BAD pathway signature, BAD pathway score, or BAD pathway expression signature) representative of the overall expression level for the plurality of genes. Preferably, the pathway score is obtained using principle components analysis. Principal components analysis can be performed to reduce data dimension into a small set of uncorrelated principal components. This set of principal components is then generated based on its ability to account for variation. For example, the pathway score can be defined as:

Σwᵢxᵢ, a weighted average expression among the plurality of BAD pathway genes, where xᵢ represents gene i expression level, wᵢ is the corresponding weight (loading coefficient) with Σwᵢ²=1, and the wᵢ values maximize the variance of Σwᵢxᵢ.

The gene expression profile may be compared to one or more reference gene expression profiles (preferably, compared to one or more reference gene expression scores) that are each indicative of an aspect of cancer prognosis (e.g., survival such as overall survival, disease- or relapse-free survival; disease progression (e.g., rate of progression of tumor growth, progression of chemo-sensitive cancer to chemo-resistant cancer, etc.) to thereby score or classify the sample and/or the sample's gene expression profile as consistent or inconsistent with a cancer prognosis. For example, the subject's gene expression profile may be predictive of (consistent with) survival or duration of survival, a pathological complete response (pCR) to treatment, or other measure of patient outcome, such as progression free interval or tumor size, cancer transition (e.g., transition from atyptical ductal hyperplasia (ADH) to ductal carcinoma in situ (DCIS) to invasive ductal carcinoma (IDS)), progression of cancer from chemosensitive to chemo-resistant, among others.

Optionally, after the subject's prognosis is assessed, the method may further comprise administering an agent that targets the BAD pathway. In some embodiments, the agent comprises one or more compounds listed in Table 2 or Table 3. Preferably, an effective amount of the agent is administered to alleviate at least one symptom of cancer in the subject.

Another aspect of the invention is a method for preparing a gene expression profile indicative of chemo-resistance or chemo-sensitivity, comprising: obtaining a biological sample from the subject, and determining the level of expression for a plurality of genes of BAD pathway in the sample, thereby preparing the gene expression profile. In some embodiments, the chemo-resistance or chemo-sensitivity comprises resistance or sensitivity to platinum-based therapy. Thus, the gene expression profile may be obtained from a subject and may be used for evaluating the sensitivity and/or resistance of cancer specimens (e.g., tumor specimens) to anti-cancer therapies, such as platinum-based therapies (monotherapy or combination therapies) for the subject. Particularly, the invention provides gene expression profiles that are indicative of a cancer's sensitivity and/or resistance to candidate therapeutic regimens, such as regimens that include platinum-based therapies.

Preferably, the plurality of genes of the BAD pathway comprises a plurality of genes listed in Table 1. In some embodiments, the plurality of BAD pathway genes is 53 BAD pathway genes, or a subset thereof. In some embodiments, the plurality of BAD pathway genes is 47 BAD pathway genes. In some embodiments, the 53 BAD pathway genes are those represented by U133Plus of Table 1. In some embodiments, the 47 BAD pathway genes are those represented by U133A of Table 1.

In some embodiments, the plurality of BAD pathway genes comprises or consists of the following 53 BAD pathway genes: MAP2K2, RAF1, HRAS, SCH1, EGFR, IRS1, PIK3CA, PIK3CB, PIK3CD, RPS6KA1, RPS6KA2, RPS6KA3, BAD, BCL2L1, BAX, AKT1, AKT2, AKT3, GNB1, GNB2, GNB3, GNB4, GNB5, GNG10, GNG11, GNG12, GNG13, GNG2, GNG3, GNG4, GNG5, GNG7, GNG8, GNGT1, GNGT2, PPM1A, PPM1B, PPM1D, PPM1F, PPM1G, PPM1L, PPM2C, PPTC7, PTPN11, GNAS, PRKAR1A, PRKAR1B, PRKAR2A, PRKAR2B, CDC2, PRKACA, PRKACB, and PRKACG.

In some embodiments, the plurality of BAD pathway genes comprises or consists of the following 47 BAD pathway genes: MAP2K2, RAF1, HRAS, SCH1, EGFR, IRS1, PIK3CA, PIK3CB, PIK3CD, RPS6KA1, RPS6KA2, RPS6KA3, BAD, BCL2L1, BAX, AKT1, AKT2, AKT3, GNB1, GNB2, GNB3, GNB5, GNG10///LOC552891, GNG11, GNG12, GNG13, GNG3, GNG4, GNG5, GNG7, GNGT1, PPM1A, PPM1B, PPM1D, PPM1F, PPM1G, PPM2C, PTPN11, GNAS, PRKAR1A, PRKAR1B, PRKAR2A, PRKAR2B, CDC2, PRKACA, PRKACB, and PRKACG.

Thus, in one aspect, the invention provides methods for preparing a gene expression profile for a biological sample (such as a tumor specimens or cultured cells), as well as methods for predicting a cancer's sensitivity or resistance to therapeutic by evaluating the subject's BAD gene expression profile, preferably expressed as a BAD pathway score (also referred to as a BAD pathway signature), and determining whether the profile is indicative of resistance or sensitivity. The sample may be a biological sample from a subject or a cell line, for example. By predicting the cancer's sensitivity or resistance to candidate therapeutic agents, this aspect of the invention thereby provides information to guide individualized cancer treatment.

The gene expression profile may be compared to one or more reference gene expression profiles (preferably, compared to one or more reference gene expression scores) that are each indicative of sensitivity or resistance to a candidate agent or combination of agents, to thereby score or classify the test sample and/or the test gene expression profile as sensitive or resistant to such agents or combinations. For example, the gene expression profile may be indicative of sensitivity or resistance to one or more of carboplatin, paclitaxel, doxetaxel, doxorubicin, topetcan, cisplatin, gemcitabine, cyclophosphamide, or a combination of two or more of the foregoing.

Optionally, in the aforementioned methods of the invention, the results of gene expression analysis are combined with results from in vitro chemosensitivity testing, to provide a more complete and/or accurate prognostic and/or predictive tool for guiding patient therapy.

In the aforementioned methods of the invention, the gene expression profile may be prepared directly from patient specimens, e.g., by a process comprising RNA extraction or isolation directly from tumor specimens, or alternatively, and particularly where specimens are amenable to culture, malignant cells may be enriched (e.g., expanded) in culture for gene expression analysis. For example, malignant cells may be enriched in culture by disaggregating or mincing the tumor specimen to prepare tumor tissue explants, and allowing one or more tumor tissue explants to form a cell culture monolayer. RNA is then extracted from the cultured cells for gene expression analysis. The resulting gene expression profile, whether prepared directly from patient tissue (e.g., tumor tissue) or prepared from cultured cells, contains gene transcript levels (or “expression levels”) for BAD pathway genes that are indicative of cancer prognosis or indicative of either chemo-resistance or chemo-sensitivity (chemo-resistance/chemo-sensitivity).

In the aforementioned methods, the gene expression profiles in some embodiments include those generally applicable to a variety of cancer types and/or therapeutic agent(s). Alternatively, or in addition, the gene expression profiles are predictive for a particular type of cancer, such as breast cancer, and/or for a particular course of treatment.

In the aforementioned methods of the invention, cultured cells may be immortalized cell lines, or may be derived directly from patient tumor specimens, for example, by enriching or expanding malignant from the tumor specimen in monolayer culture, and suspending the cultured cells for testing and/or RNA isolation. The resulting gene expression profiles can then be independently validated in patient test populations having available gene expression data and corresponding clinical data, including information regarding the treatment regimen and outcome of treatment. This aspect of the invention reduces the length of time and quantity of patient samples needed for identifying and validating such gene expression signatures.

Another aspect of the invention concerns a method for treating cancer in a subject, comprising administering an agent that targets the BAD pathway. In some embodiments, the agent comprises one or more compounds listed in Table 2 or Table 3. Preferably, an effective amount of the agent is administered to alleviate at least one symptom of cancer in the subject.

Another aspect of the invention is a method for treating cancer in a subject, comprising administering an agent that targets the BAD pathway to the subject, wherein the subject is predetermined to have a poor cancer prognosis based on the level of expression of a plurality of genes of the BAD pathway. Another aspect of the invention is a method for treating cancer in a subject, comprising: (a) assessing the prognosis of cancer in the subject, comprising comparing the level of expression of a plurality of genes of the BAD pathway in a sample from the subject to a reference BAD pathway gene expression level; and (b) administering an agent that targets the BAD pathway to the subject if the subject is assessed to have a poor or undesirable prognosis. In some embodiments, the agent comprises one or more of those listed in Table 2 or Table 3. Preferably, an effective amount of the agent is administered to alleviate at least one symptom of cancer in the subject.

Another aspect of the invention is a method for treating chemo-sensitive cancer in a subject, comprising administering a chemotherapeutic agent to the subject, wherein the cancer is predetermined to be chemo-sensitive based on the level of expression of a plurality of genes of the BCL2 antagonist of cell death (BAD) pathway. Another aspect of the invention is a method for treating cancer in a subject, comprising: (a) assessing the chemo-sensitivity or chemo-resistance of the cancer, comprising comparing the level of expression of a plurality of genes of the BCL2 antagonist of cell death (BAD) pathway in a sample of the cancer to a reference BAD pathway gene expression level; and (b) administering a chemotherapeutic agent to the subject if the cancer is determined to be chemo-sensitive based on the assessment of (a). Preferably, an effective amount of the chemotherapeutic agent is administered to alleviate at least one symptom of cancer in the subject.

In other aspects, the invention provides computer systems, kits, and other compositions of matter (e.g., microarray, bead set, probe set) for generating gene expression profiles that are useful for determining prognosis or for predicting a cancer's response to a chemotherapeutic agent, for example, in connection with the methods of the invention.

### BRIEF DESCRIPTION OF THE DRAWINGS

For a fuller understanding of the invention, reference should be made to the following detailed description, taken in connection with the accompanying drawings, in which:

FIG. 1A is a graph of the survival of 143 OVCA patients showing low PC1 versus high PC1 (Hope sig. gene, 98 probe sets, cutoff=median)

FIG. 1B is a graph of the survival of 143 OVCA patients showing CR versus IR.

FIG. 1C is a graph of the survival of 104 OVCA patients (CR) showing low PC1 versus high PC1 (Hope sig. gene, 98 probe sets, cutoff=median)

FIG. 1D is a graph of the survival of 39 OVCA patients (IR) showing low PC1 versus high PC1 (Hope sig. gene, 98 probe sets, cutoff=median)

FIG. 1E is a graph of the survival of 143 OVCA patients (CRIR*PC1) (Hope sig. gene, 98 probe sets, cutoff=median)

FIG. 1F is a graph of the survival of 142 OVCA patients showing optimal versus suboptimal.

FIG. 1G is a graph of the survival of 74 OVCA patients (optimal) showing low PC1 versus high PC1 (Hope sig. gene, 98 probe sets, cutoff=median)

FIG. 1H is a graph of the survival of 68 OVCA patients (sub-optimal) showing low PC1 versus high PC1 (Hope sig. gene, 98 probe sets, cutoff=median)

FIG. 1I is a graph of the survival of 143 OVCA patients (optimal/sub-optimal*PC1) (Hope sig. gene, 98 probe sets, cutoff=median)

FIG. 2A is a graph showing the results for prediction (derived from Moffitt dataset) using unstandardized expression data (scale=F) for 240 patients with ovarian cancer treated in Australia (the Australian OVCA dataset).

FIG. 2B is a graph showing the results for prediction (derived from Moffitt dataset) using standardized expression data (scale=T) for 240 patients with ovarian cancer treated in Australia (the Australian OVCA dataset).

FIG. 2C is a graph showing the results for association using unstandardized expression data (scale=F) for 240 patients with ovarian cancer treated in Australia (the Australian OVCA dataset).

FIG. 2D is a graph showing the results for association using standardized expression data (scale=T) for 240 patients with ovarian cancer treated in Australia (the Australian OVCA dataset).

FIG. 2E is a graph showing the correlation of weight (loading coefficient) between unstandardized versus standardized expression data for 240 patients with ovarian cancer treated in Australia (the Australian OVCA dataset).

FIG. 3A is a graph showing the results for prediction (derived from Moffitt dataset) using unstandardized expression data (scale=F) for 286 patients with breast cancer and followed for both relapse-free survival and also distant metastasis free survival (the breast cancer 286 dataset).

FIG. 3B is a graph showing the results for prediction (derived from Moffitt dataset) using standardized expression data (scale=T) for 286 patients with breast cancer and followed for both relapse-free survival and also distant metastasis free survival (the breast cancer 286 dataset).

FIG. 3C is a graph showing the results for association using unstandardized expression data (scale=F) for 286 patients with breast cancer and followed for both relapse-free survival and also distant metastasis free survival (the breast cancer 286 dataset).

FIG. 3D is a graph showing the results for association using standardized expression data (scale=T) for 286 patients with breast cancer and followed for both relapse-free survival and also distant metastasis free survival (the breast cancer 286 dataset).

FIG. 3E is a graph showing the correlation of weight (loading coefficient) between unstandardized versus standardized expression data for 286 patients with breast cancer and followed for both relapse-free survival and also distant metastasis free survival (the breast cancer 286 dataset).

FIG. 4A is a graph showing the results for association using unstandardized expression data (scale=F) for the 50 brain cancers.

FIG. 4B is a graph showing the results for association using standardized expression data (scale=T) for the 50 brain cancers.

FIG. 4C is a graph showing the correlation of weight (loading coefficient) between unstandardized versus standardized expression data for the 50 brain cancers.

FIG. 5A is a graph showing the results for prediction (derived from Moffitt dataset) using unstandardized expression data (scale=F) for the 182 brain cancers.

FIG. 5B is a graph showing the results for prediction (derived from Moffitt dataset) using standardized expression data (scale=T) for the 182 brain cancers.

FIG. 5C is a graph showing the results for association using unstandardized expression data (scale=F) for the 182 brain cancers.

FIG. 5D is a graph showing the results for association using standardized expression data (scale=T) for the 182 brain cancers.

FIG. 5E is a graph showing the correlation of weight (loading coefficient) between unstandardized versus standardized expression data for the 182 brain cancers.

FIG. 6A is a graph showing the results for prediction (derived from Moffitt dataset) using unstandardized expression data (scale=F) for the 130 lung cancers.

FIG. 6B is a graph showing the results for prediction (derived from Moffitt dataset) using standardized expression data (scale=T) for the 130 lung cancers.

FIG. 6C is a graph showing the results for association using unstandardized expression data (scale=F) for the 130 lung cancers.

FIG. 6D is a graph showing the results for association using standardized expression data (scale=T) for the 130 lung cancers.

FIG. 6E is a graph showing the correlation of weight (loading coefficient) between unstandardized versus standardized expression data for the 130 lung cancers.

FIG. 7A is a graph showing the results for prediction (derived from Moffitt dataset) using unstandardized expression data (scale=F) for 205 patients with colon cancer treated at Moffitt Cancer Center (the MCC colon dataset).

FIG. 7B is a graph showing the results for prediction (derived from Moffitt dataset) using standardized expression data (scale=T) for 205 patients with colon cancer treated at Moffitt Cancer Center (the MCC colon dataset).

FIG. 7C is a graph showing the results for association using unstandardized expression data (scale=F) for 205 patients with colon cancer treated at Moffitt Cancer Center (the MCC colon dataset).

FIG. 7D is a graph showing the results for association using standardized expression data (scale=T) for 205 patients with colon cancer treated at Moffitt Cancer Center (the MCC colon dataset).

FIG. 7E is a graph showing the correlation of weight (loading coefficient) between unstandardized versus standardized expression data for 205 patients with colon cancer treated at Moffitt Cancer Center (the MCC colon dataset).

FIG. 8A is a graph showing the association of the BAD pathway score with the transition from normal to atypical hyperplasia to invasive cancer for the 33 endometrial samples (the MCC endometrial dataset).

FIG. 8B is a graph showing the differences in mean levels of the 33 endometrial samples (the MCC endometrial dataset).

FIG. 9 is a graph showing the BAD pathway score was associated with the transition from atypical ductal hyperplasia (ADH) to ductal carcinoma in situ (DCIS) to invasive ductal carcinoma (IDC) for the 61 breast samples (ADH, DCIS, IDC)(the Ma et al. dataset).

FIG. 10 is a graph showing the BAD pathway score was associated with the transition from normal breast tissue to ductal carcinoma in situ (DCIS) to invasive ductal carcinoma (IDC) for the 197 breast samples (normal, DCIS, and IDC)(the MCC 197 dataset).

FIG. 11 is a graph showing the BAD score was associated with relapse-free survival for the Chanrion Tamoxifen-Treated Primary Breast Cancer Study (relapse-free versus relapse)(Charion dataset).

FIG. 12 shows BAD pathway genes associated with induced cisplatin-resistance. Thermometers indicate those genes that demonstrated a positive (red, with thermometer extending upward from number) and negative (blue, with thermometer extending downward from number) correlation between expression and increased cisplatin-resistance (EC50) (P<0.001 for pathway enrichment): red thermometers identify those genes with increasing expression associated with increasing OVCA cisplatin-resistance, and blue thermometers identify those genes with decreasing expression associated with increasing OVCA cisplatin-resistance. Numbers 1-8 at thermometer base identify the cell line (1=T8, 2=OVCAR5, 3=OV2008, 4=IGROV1, 5=C13, 6=A2780S, 7=A2780CP, 8=A2008) that demonstrated changes in expression of that gene with increasing cisplatin-resistance.

FIG. 13A-E show that BAD-protein phosphorylation is associated with platinum resistance. FIGS. 13A-13D show cisplatin EC50 results and percent expression of phosphorylated-BAD at serine-155 (P-BAD155), non-phosphorylated BAD (NP-BAD155), total BAD, and PP2C (PPM1A) in ovarian cancer cell lines (A2780S, A2780CP, A2008, and C13, respectively) measured by MTS and immunofluorescence, respectively. FIG. 13E shows percent expression of P-BAD at serine-155, -136, and -112 by immunofluorescence in an independent set of 148 primary advanced-stage OVCA samples, including platinum-sensitive/complete responders (CR, n=80) and platinum-resistant/incomplete-responders (IR, n=68). Error bars indicate standard error of the mean.

FIGS. 14A-D show that modulation of BAD-protein phosphorylation status influences cisplatin sensitivity. In FIGS. 14A and 14B, OVCA cell lines A2780S and A2780CP, respectively, were transfected with Flag vectors expressing wild-type BAD (WT) or BAD harboring serine (S) to alanine point mutations in serine-112, -136, or -155 (S112A, S136A, S155A). These S to A phosphorylation site mutations prevent phosphorylation of the BAD protein. Transfected cells were treated with vehicle or 1 μM (A2780S) or 10 μM (A2780CP) cisplatin for 48 hours and evaluated for the presence of apoptotic nuclei. FIG. 14C is a Western blot showing depletion of PP2C and PKA by siRNA. Controls included a non-targeting siRNA (NT). GAPDH was used as a loading control. FIG. 14D shows percent apoptotic nuclei in A2780S cells in the presence of 1 μM cisplatin after siRNA depletion of PKA and PP2C. Error bars indicate standard error of the mean.

FIGS. 15A-I show that high BAD-pathway signature principal component analysis (PCA) score is associated with favorable clinical outcome. Kaplan-Meier curves depicting the association between BAD-pathway signature PCA score and overall survival from cancer. FIGS. 15A-C: North American ovarian cancer dataset (*MCC). ̂Information available for 141 of 142 samples. FIG. 15D: Australian ovarian cancer dataset (Tothill et al.¹⁸). FIG. 15E: colon cancer dataset (***MCC). FIGS. 15F and 15G: brain cancer dataset (Nutt et al.¹⁹ and Lee et al.²⁰, respectively). FIGS. 15H and 15I: disease-free survival from breast cancer (Wang et al²² and Chanrion et al.²³, respectively). The numbers at risk are shown at the bottom of graphs. Log-rank test P values indicate significance. CR, complete response; IR, incomplete response; O, optimal; S, suboptimal.

FIG. 16 is a graph showing mean BAD pathway score (mean PC1 score) versus platinum response in 142 samples from ovarian cancer patients who experienced either a complete response (CR) or an incomplete response (IR) (p=0.26).

FIG. 17 is a graph showing mean BAD pathway score (mean PC1 score) versus platinum response in 178 formalin-fixed paraffin-embedded (FFPE) tissue samples from patients who experienced either a CR or IR (p=0.0954).

FIG. 18 is a graph showing BAD pathway score (PC1 score) versus platinum response in 178 FFPE tissue samples from patients who experienced either a CR or IR (p=0.0954).

### DETAILED DISCLOSURE OF THE INVENTION

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

The BAD pathway is a critical driver of cellular apoptosis control. Using genome-wide expression analysis, the inventors recently identified the BAD pathway to be a key component of ovarian cancer (OVCA) chemo-sensitivity. They evaluated the utility of panels of BAD pathway genes as determinants of survival for patients with cancer, and as determinants of chemo-resistance/chemo-sensitivity of cancers.

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons, Inc., New York, 2000) and Casarett and Doull's Toxicology The Basic Science of Poisons, C. Klaassen, ed., 6th edition (2001).

In the methods of the invention, a sample is analyzed to obtain a BAD pathway gene expression profile based on a plurality of BAD pathway genes. This can be achieved any number of ways. One method that can be used is to isolate RNA (e.g., total RNA) from a cellular sample and use a publicly available microarray systems to analyze the gene expression profile from the cellular sample. One microarray that may be used is Affymetrix Human U133A chip. One of skill in the art can follow the standard directions that come with a commercially available microarray. Other types of microarrays may be used, for example, microarrays using RT-PCR for measurement. Other sources of microarrays include, but are not limited to, Stratagene (e.g., Universal Human Microarray), Genomic Health (e.g., Oncotype DX chip), Clontech (e.g., Atlas™ Glass Microarrays), and other types of Affymetrix microarrays. In some embodiments, customized microarrays, which include the particular set of genes that are particularly suitable for prediction, can be used.

Once a BAD pathway gene expression profile has been obtained from the sample, then it can be used to compare with one or more reference gene expression profiles to determine an aspect of prognosis or chemotherapy responsivity (chemo-sensitivity/chemo-resistance). Preferably, the BAD pathway gene expression profile is expressed as a BAD pathway signature (BAD pathway score); likewise, preferably, the one or more reference gene expression profiles are expressed as a reference BAD pathway signature (reference BAD pathway score). In some embodiments, the BAD pathway score is a threshold or cutoff in which a BAD pathway score above the cutoff is indicative of an aspect of prognosis or cancer responsivity as opposed to a different predicted outcome (e.g., the opposite outcome) if the BAD pathway score is below the cutoff. The cutoff may be a mean or median pathway score. For example, if the sample's BAD pathway score is higher than the reference pathway score (i.e., a high BAD pathway score or desirable prognosis), it may be indicative of one aspect of cancer prognosis (such as a survival advantage, prediction of no relapse, no disease progression or slower rate of cancer progression) relative to a BAD pathway score that is lower than the reference pathway score (i.e., a low BAD pathway score or poor or undesirable prognosis). It will be appreciated to those skilled in the art, however, that, depending upon how the BAD pathway score is obtained (the reference pathway score and the sample pathway score), a sample pathway score that is either “higher” or “lower” than the cutoff may be predictive of the same clinical outcome (e.g., survival, disease progression, relapse, chemoresistance/chemosensitivity). For example, a pathway score obtained by one method may be higher than a reference cutoff, but a pathway score obtained by a different method may be lower than the reference cutoff. The pathway score can be a relative value and the invention is not limited in this respect. For example, depending upon the method by which the pathway score is obtained, a “high” pathway score (higher than the reference cutoff) may represent a good (desirable) prognosis or it may represent a poor (undesirable) prognosis. Likewise, depending upon the method by which the pathway score is obtained, a “low” pathway score (lower than the reference cutoff) may represent a good prognosis or a poor prognosis. This aspect of the comparison between the sample pathway score and the reference pathway score is not critical to the invention.

Preferably, the pathway score is obtained using principle components analysis. Principal components analysis can be performed to reduce data dimension into a small set of uncorrelated principal components. This set of principal components is then generated based on its ability to account for variation. For example, the pathway score can be defined as:

Σwᵢxᵢ, a weighted average expression among the plurality of BAD pathway genes, where xᵢ represents gene i expression level, wᵢ is the corresponding weight (loading coefficient) with Σwᵢ²=1, and the wᵢ values maximize the variance of Σwᵢxᵢ.

### Biological Samples

In some embodiments, the methods of the invention include determining the expression level of genes in a biological sample (for example, a tumor sample). In some embodiments, the methods comprise the step of surgically removing a tumor sample from a subject, obtaining a tumor sample from the subject, or providing a tumor sample from the subject. In some embodiments, the sample contains at least 40%, 50%, 60%, 70%, 80% or 90% tumor cells. In some embodiments, the tumor sample is a frozen sample. In one embodiments, the sample is one that was frozen within less than 5, 4, 3, 2, 1, 0.75, 0.5, 0.25, 0.1, 0.05 or less hours after extraction from a subject. Preferred frozen sample include those stored in liquid nitrogen or at a temperature of about −80 C or below.

### Gene Expression

The expression of the BAD pathway genes may be determined using any methods known in the art for assaying gene expression. Gene expression may be determined by measuring RNA for the genes. In a preferred embodiment, an mRNA transcript of a gene may be detected for determining the expression level of the gene. Based on the sequence information provided by the GenBank™ database entries, the genes can be detected and expression levels measured using techniques well known to one of ordinary skill in the art. For example, sequences within the sequence database entries corresponding to polynucleotides of the genes can be used to construct probes for detecting mRNAs by, e.g., Northern blot hybridization analyses. The hybridization of the probe to a gene transcript in a subject biological sample can also be carried out on a DNA array. The use of an array is preferable for detecting the expression level of a plurality of the genes. As another example, the sequences can be used to construct primers for specifically amplifying the polynucleotides in, e.g., amplification-based detection methods such as reverse-transcription based polymerase chain reaction (RT-PCR). Furthermore, the expression level of the genes can be analyzed based on the biological activity or quantity of proteins encoded by the genes.

In some embodiments, cancer tissue is added to a chilled tissue pulverizer, such as to a BioPulverizer H tube (Bio101 Systems, Carlsbad, Calif.). Lysis buffer, such as from the Qiagen Rneasy Mini kit, is added to the tissue and homogenized. Devices such as a Mini-Beadbeater (Biospec Products, Bartlesville, Okla.) may be used. Tubes may be spun briefly as needed to pellet the garnet mixture and reduce foam. The resulting lysate may be passed through syringes, such as a 21 gauge needle, to shear DNA. Total RNA may be extracted using commercially available kits, such as the Qiagen RNeasy Mini kit. The samples may be prepared and arrayed using Affymetrix U133 plus 2.0 GeneChips or Affymetrix U133A GeneChips, for example.

In some embodiments, deter wining the expression level of multiple genes in a sample from the subject comprises extracting a nucleic acid sample from the sample from the subject, preferably an mRNA sample. In one embodiment, the expression level of the nucleic acid is determined by hybridizing the nucleic acid, or amplification products thereof, to a DNA microarray. Amplification products may be generated, for example, with reverse transcription, optionally followed by PCR amplification of the products.

### Methods for Assessing Cancer Prognosis and Predicting Response to Cancer Treatment

The invention provides methods for preparing gene expression profiles for samples such as tumor specimens, as well as methods for prognosis and methods for evaluating a cancer's sensitivity and/or resistance to one or more therapeutic agents or combinations of agents. For example, the gene expression profile generated for a tumor specimen, or cultured cells derived therefrom, can be evaluated for the presence of one or more indicative BAD pathway gene expression signatures. The gene expression signatures are indicative of an aspect of cancer prognosis, indicative of a response to a treatment regimen, or both. In this way, the methods of the invention provide information to guide a physician in designing/administering an individualized therapeutic regimen for a cancer patient.

The patient generally is one with a cancer or neoplastic condition. The patient may suffer from cancer of essentially any tissue or organ, including but not limited to breast, ovaries, lung, colon, skin, prostate, kidney, endometrium, nasopharynx, pancreas, head and neck, kidney, and brain, among others. The patient may be inflicted with a carcinoma or sarcoma. The patient may have a solid tumor of epithelial origin. The cancer specimen may be obtained from the patient by surgery, or may be obtained by biopsy, such as a fine needle biopsy or other procedure prior to the selection/initiation of therapy. In certain embodiments, the cancer is breast cancer, including preoperative or post-operative breast cancer. In certain embodiments, the patient has not undergone treatment to remove the breast tumor.

The cancer may be primary or recurrent, and may be of any type (as described above), stage (e.g., Stage I, II, III, or IV or an equivalent of other staging system), and/or histology (e.g., serous adenocarcinoma, endometroid adenocarcinoma, mucinous adenocarcinoma, undifferentiated adenocarcinoma, transitional cell adenocarcinoma, or adenocarcinoma, etc.). The patient may be of any age, sex, performance status, and/or extent and duration of remission.

In certain embodiments, the patient is a candidate for treatment with one or more platinum-based therapies. In some embodiments, the patient is a candidate for treatment with carboplatin, paclitaxel, doxetaxel, doxorubicin, topetcan, cisplatin, gemcitabine, cyclophosphamide, or a combination of two or more of the foregoing.

In some embodiments, the subject has undergone a treatment for cancer before and/or after a sample is obtained from the subject. For example, the cancer treatment may include primary surgery, chemotherapy (for example, platinum-based therapy), or both. In some embodiments, the subject has experienced a complete response (CR) to the cancer treatment before or after the sample is obtained from the subject. In other embodiments, the subject has experienced an incomplete response (IR) to the cancer treatment before or after the sample is obtained from the subject.

In some embodiments, the subject has undergone primary surgical cytoreduction (debulking) before and/or after a sample is obtained from the subject. The debulking may be obtimal debulking (left with residual disease less than 1 centimeter in greatest diameter) or sub-optimal debulking (left with residual disease greater than 1 centimeter in greatest diameter), for example.

The BAD pathway gene expression profile can be determined for a tumor tissue or cell sample, such as a tumor sample removed from the patient by surgery or biopsy. The tumor sample may be “fresh,” in that it was removed from the patent within about five days of processing, and remains suitable or amenable to culture. In some embodiments, the tumor sample is not “fresh,” in that the sample is not suitable or amenable to culture. The sample may be frozen after removal from the patient, and preserved for later RNA isolation. The sample for RNA isolation may be a formalin-fixed paraffin-embedded (FFPE) tissue.

In some embodiments, the malignant cells are enriched or expanded in culture by forming a monolayer culture from tumor sample explants. For example, cohesive multicellular particulates (explants) are prepared from a patient's tissue sample (e.g., a biopsy sample or surgical specimen) using mechanical fragmentation. This mechanical fragmentation of the explant may take place in a medium substantially free of enzymes that are capable of digesting the explant. Some enzymatic digestion may take place in certain embodiments, such as for ovarian or colorectal tumors.

Where it is desirable to expand and/or enrich malignant cells in culture relative to non-malignant cells that reside in the tumor, the tissue sample can be systematically minced using two sterile scalpels in a scissor-like motion, or mechanically equivalent manual or automated opposing incisor blades. The process for enriching or expanding malignant cells in culture is described in U.S. Pat. Nos. 5,728,541, 6,900,027, 6,887,680, 6,933,129, 6,416,967, 7,112,415, 7,314,731, and 7,501,260 (all of which are hereby incorporated by reference in their entireties). The process may further employ the variations described in US Published Patent Application Nos. 2007/0059821 and 2008/0085519, both of which are hereby incorporated by reference in their entireties.

In preparing the gene expression profile, RNA can be extracted from tumor tissue or cultured cells by any known method. For example, RNA may be purified from cells using a variety of standard procedures as described, for example, in RNA Methodologies, A laboratory guide for isolation and characterization, 2nd edition, 1998, Robert E. Farrell, Jr., Ed., Academic Press. In addition, there are various products commercially available for RNA isolation which may be used. Total RNA or polyA+ RNA may be used for preparing gene expression profiles in accordance with the invention.

The gene expression profile can then generated for the samples using any of various techniques known in the art, and described in detail elsewhere herein. Such methods generally include, without limitation, hybridization-based assays, such as microarray analysis and similar formats (e.g., Whole Genome DASL™ Assay, Illumina, Inc.), polymerase-based assays, such as RT-PCR (e.g., Tagman™), flap-endonuclease-based assays (e.g., Invader™), as well as direct mRNA capture with branched DNA (QuantiGene™) or Hybrid Capture™ (Digene).

The gene expression profile contains gene expression levels for a plurality of BAD pathway genes whose expression levels are predictive or indicative of an aspect of cancer prognosis or the cancer's response to one or a combination of therapeutic agents. As used herein, the term “gene,” refers to a DNA sequence expressed in a sample as an RNA transcript, and may be a full-length gene (protein encoding or non-encoding) or an expressed portion thereof such as expressed sequence tag or “EST.”

The plurality of BAD pathway genes utilized may be differentially expressed in chemo-sensitive samples versus chemo-resistant samples, or in positive prognosis samples or negative prognosis samples, as described below. As used herein, “differentially expressed” means that the level or abundance of an RNA transcript (or abundance of an RNA population sharing a common target (or probe-hybridizing) sequence, such as a group of splice variant RNAs) is significantly higher or lower in a sample as compared to a reference level (e.g., a chemo-resistant sample). For example, the level of the RNA or RNA population may be higher or lower than a reference level. The reference level may be the level of the same RNA or RNA population in a control sample or control population (e.g., a mean level for a chemo-resistant sample), or may represent a cut-off or threshold level.

The BAD gene expression profile generally contains the expression levels for at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 or more BAD pathway genes. As discussed, these expression levels represent the gene expression state of a sample (such as a patient's malignant cells or tumor), and are evaluated for the presence of one or more gene signatures (e.g., BAD pathway score) indicative of a subject's prognosis or indicative of the cancer's sensitivity and/or resistance to chemotherapeutic agents. Preferably, the raw expression levels of the plurality of BAD pathway genes are expressed as an overall expression level or BAD pathway score.

Various classification schemes are known for classifying samples between two or more classes or groups, and these include, without limitation: Principal Components Analysis (PCA), Naive Bayes, Support Vector Machines, Nearest Neighbors, Decision Trees, Logistic, Artificial Neural Networks, and Rule-based schemes. In addition, the predictions from multiple models can be combined to generate an overall prediction. For example, a “majority rules” prediction may be generated from the outputs of a Naive Bayes model, a Support Vector Machine model, and a Nearest Neighbor model.

Preferably, the pathway score is obtained using principle components analysis. Principal components analysis can be performed to reduce data dimension into a small set of uncorrelated principal components. This set of principal components is then generated based on its ability to account for variation. For example, the pathway score can be defined as:

Σwᵢxᵢ, a weighted average expression among the plurality of BAD pathway genes, where xᵢ represents gene i expression level, wᵢ is the corresponding weight (loading coefficient) with Σwᵢ²=1, and the wᵢ values maximize the variance of Σwᵢxᵢ.

Generally, the gene expression profiles for samples are scored or classified as signatures consistent or inconsistent with an aspect of cancer prognosis such as survival or clinical outcome, or as chemo-sensitive signatures or chemo-resistant signatures. These classifications may include stratified or continuous intermediate classifications or scores reflective of cancer prognosis or chemo-sensitivity/chemo-resistance. The signatures may be stored in a database and correlated to patient gene expression profiles in response to user inputs.

After comparing the sample gene expression signature (e.g., a patient's gene expression profile) to a reference gene expression signature, the sample can be classified as, or for example, having a positive (desirable) or negative (undesirable or poor) prognosis profile, or of having a chemo-sensitive profile or a chemo-resistant profile, or having a probability of having such profiles. The classification may be determined computationally based upon known methods as described above. The result of the computation may be displayed on a computer screen or presented in a tangible form, for example, as a probability (e.g., from 0 to 100%) of the patient responding to a given treatment. The report will aid a physician in selecting a course of treatment for the cancer patient. For example, in certain embodiments of the invention, the patient's gene expression profile will be determined, on the basis of probability, to be one of survival (e.g., 5-year survival), lack of survival, disease progression, lack of disease progression, chemo-sensitive, or chemo-resistant, and the patient will be subsequently treated accordingly. In some situations, this information can allow a physician to either include or exclude a candidate treatment for the patient (such as a chemotherapeutic agent and/or agent that targets the BAD pathway), perhaps sparing the patient unnecessary toxicity.

The methods of the invention aid the prediction of an outcome of treatment. That is, the gene expression signatures are each predictive of a clinical outcome. The outcome may be quantified in a number of ways. For example, the outcome may be an objective response, a clinical response, or a pathological response to a candidate treatment. The outcome may be determined based upon the techniques for evaluating response to treatment of solid tumors as described in Therasse et al., New Guidelines to Evaluate the Response to Treatment in Solid Tumors, J. of the National Cancer Institute 92(3):205-207 (2000), which is hereby incorporated by reference in its entirety. For example, the outcome may be survival (including overall survival or the duration of survival), progression-free interval, or survival after recurrence. The timing or duration of such events may be determined from about the time of diagnosis or from about the time treatment (e.g., chemotherapy) is initiated. Alternatively, the outcome may be based upon a reduction in tumor size, tumor volume, or tumor metabolism, or based upon overall tumor burden, or based upon levels of serum markers especially where elevated in the disease state (e.g., PSA). The outcome in some embodiments may be characterized as a complete response, a partial or incomplete response, stable disease, and progressive disease. The methods of the invention may further comprise selecting a treatment for the patient for which a more favorable prognosis can be obtained, based on the subject's BAD pathway signature.

In some embodiments, the BAD pathway gene signature is indicative of a pathological complete response upon treatment with a particular candidate agent or combination (as already described). A pathological complete response, e.g., as determined by a pathologist following examination of tissue (e.g., breast or nodes in the case of breast cancer) removed at the time of surgery, generally refers to an absence of histological evidence of invasive tumor cells in the surgical specimen.

The present invention may further comprise conducting chemo-response testing with a panel of chemotherapeutic agents on cultured cells from a cancer patient, to thereby add additional predictive value. That is, the presence of one or more gene expression signatures in tumor cells, and the in vitro chemoresponse results for the tumor specimen, are used to predict an outcome of treatment (e.g., survival, pCR, etc.). For example, where the BAD pathway gene expression profile and chemoresponse test both indicate that a tumor is sensitive or resistant to a particular treatment, the predictive value of the method may be particularly high. Likewise, where the BAD pathway gene expression profile is consistent or inconsistent with an aspect of cancer prognosis (such as survival) and the chemoresponse test indicates that a tumor is sensitive or resistant to a particular treatment, the predictive value of the method may be particularly high.

Several in vitro chemoresponse systems are known and art, and some are reviewed in Fruehauf et al., In vitro assay-assisted treatment selection for women with breast or ovarian cancer, Endocrine-Related Cancer 9: 171-82 (2002). In certain embodiments, the chemoresponse assay is as described in U.S. Pat. Nos. 5,728,541, 6,900,027, 6,887,680, 6,933,129, 6,416,967, 7,112,415, 7,314,731, 7,501,260 (all of which are hereby incorporated by reference in their entireties). The chemoresponse method may further employ the variations described in US Published Patent Application Nos. 2007/0059821 and 2008/0085519, both of which are hereby incorporated by reference in their entireties.

### Gene Expression Assay Formats

BAD pathway gene expression profiles, including patient gene expression profiles and reference gene expression profiles may be prepared according to any suitable method for measuring gene expression. That is, the profiles may be prepared using any quantitative or semi-quantitative method for determining RNA transcript levels in samples. Such methods include polymerase-based assays, such as RT-PCR, Taqman™, hybridization-based assays, for example using DNA microarrays or other solid support (e.g., Whole Genome DASL™ Assay, Illumine, Inc.), nucleic acid sequence based amplification (NASBA), flap endonuclease-based assays, as well as direct mRNA capture with branched DNA (QuantiGene™) or Hybrid Capture™ (Digene). The assay format, in addition to determining the gene expression levels for a combination of BAD pathway genes, can also allow for the control of, inter alia, intrinsic signal intensity variation between tests. Such controls may include, for example, controls for background signal intensity and/or sample processing, and/or other desirable controls for gene expression quantification across samples. For example, expression levels between samples may be controlled by testing for the expression level of one or more genes that are not differentially expressed between chemo-sensitive and chemo-resistant cells, or which are generally expressed at similar levels across the population. Such genes may include constitutively expressed genes, many of which are known in the art. Exemplary assay formats for determining gene expression levels, and thus for preparing gene expression profiles are described herein.

A nucleic acid sample is typically in the form of mRNA or reverse transcribed mRNA (cDNA) isolated from a biological sample such as a tumor sample or a derived cultured cell population. In some embodiments, the nucleic acids in the sample may be cloned or amplified, generally in a manner that does not bias the representation of the transcripts within a sample. In some embodiments, it may be preferable to use total RNA or polyA+ RNA as a source without cloning or amplification, to avoid additional processing steps.

As is apparent to one of skill in the art, nucleic acid samples used in the methods of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology, Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or specimen of interest. Such samples also include DNA amplified from the cDNA, and RNA transcribed from the amplified DNA.

In determining a cancer's gene expression profile, a hybridization-based assay may be employed. Nucleic acid hybridization involves contacting a probe and a target sample under conditions where the probe and its complementary target sequence (if present) in the sample can form stable hybrid duplexes through complementary base pairing. The nucleic acids that do not form hybrid duplexes may be washed away leaving the hybridized nucleic acids to be detected, typically through detection of an attached detectable label. It is generally recognized that nucleic acids may be denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In some embodiments, hybridization is performed at low stringency, such as 6×SSPET at 37 degrees C. (0.005% Triton X-100), to ensure hybridization, and then subsequent washes are performed at higher stringency (e.g., 1×SSPET at 37 degrees C.) to eliminate mismatched hybrid duplexes. Successive washes may be performed at increasingly higher stringency (e.g., down to as low as 0.25×SSPET at 37 degrees C. to 50 degrees C.) until a desired level of hybridization specificity is obtained. Stringency can also be increased by addition of agents such as formamide. Hybridization specificity may be evaluated by comparison of hybridization to the test probes with hybridization to the various controls that may be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. The hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

The hybridized nucleic acids are typically detected by detecting one or more labels attached to the sample nucleic acids. The labels may be incorporated by any of a number of means well known to those of skill in the art. See WO 99/32660.

Numerous hybridization assay formats are known, and which may be used in accordance with the invention. Such hybridization-based formats include solution-based and solid support-based assay formats. Solid supports containing oligonucleotide probes designed to detect differentially expressed genes (e.g., BAD pathway genes) can be filters, polyvinyl chloride dishes, particles, beads, microparticles or silicon or glass based chips, etc. Any solid surface to which oligonucleotides can be bound, either directly or indirectly, either covalently or non-covalently, may be used. Bead-based assays are described, for example, in U.S. Pat. Nos. 6,355,431, 6,396,995, and 6,429,027, which are hereby incorporated by reference. Other chip-based assays are described in U.S. Pat. Nos. 6,673,579, 6,733,977, and 6,576,424, which are hereby incorporated by reference.

An exemplary solid support is a high density array or DNA chip, which may contain a particular oligonucleotide probes at predetermined locations on the array. Each predetermined location may contain more than one molecule of the probe, but each molecule within the predetermined location has an identical probe sequence. Such predetermined locations are termed features. Probes corresponding to BAD pathway genes may be attached to single or multiple solid support structures, e.g., the probes may be attached to a single chip or to multiple chips to comprise a chip set. An exemplary chip format is that of the U133A or U95A gene chips (Affymetrix).

Oligonucleotide probe arrays for determining gene expression can be made and used according to any techniques known in the art (see for example, Lockhart et al. (1996), Nat Biotechnol 14:1675-1680; McGall et al. (1996), Proc Nat Acad Sci USA 93:13555-13460). Such probe arrays may contain the oligonucleotide probes necessary for determining a cancer's BAD pathway gene expression profile. Thus, such arrays may contain oligonucleotide designed to hybridize to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or more genes of the BAD pathway. In some embodiments, the array contains probes designed to hybridize to all or nearly all of the genes contributing to the BAD pathway signature (score). In still other embodiments, arrays are constructed that contain oligonucleotides designed to detect all or nearly all of the genes contributing to the BAD pathway signature on a single solid support substrate, such as a chip or a set of beads. In some embodiments, the array, bead set, or probe set may contain, in some embodiments, no more than 3000 probes, no more than 2000 probes, no more than 1000 probes, no more than 500 probes, no more than 400 probes, no more than 300 probes, or no more than 200 probes so as to embody a custom probe set for determining gene expression signatures in accordance with the invention.

Probes based on the sequences of the genes described herein for preparing expression profiles may be prepared by any suitable method. Oligonucleotide probes, for hybridization-based assays, will be of sufficient length or composition (including nucleotide analogs) to specifically hybridize only to appropriate, complementary nucleic acids (e.g., exactly or substantially complementary RNA transcripts or cDNA). Typically the oligonucleotide probes will be at least about 10, 12, 14, 16, 18, 20 or 25 nucleotides in length. In some cases, longer probes of at least 30, 40, or 50 nucleotides may be desirable. In some embodiments, complementary hybridization between a probe nucleic acid and a target nucleic acid embraces minor mismatches (e.g., one, two, or three mismatches) that can be accommodated by reducing the stringency of the hybridization media to achieve the desired detection of the target polynucleotide sequence. Of course, the probes may be perfect matches with the intended target probe sequence, for example, the probes may each have a probe sequence that is perfectly complementary to a target BAD pathway gene sequence.

A probe is a nucleic acid capable of binding to a target nucleic acid of complementary sequence through one or more types of chemical bonds, usually through complementary base pairing, usually through hydrogen bond formation. A probe may include natural (i.e., A, G, U, C, or T) or modified bases (7-deazaguanosine, inosine, etc.), or locked nucleic acid (LNA). In addition, the nucleotide bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages.

When using hybridization-based assays, in may be necessary to control for background signals. The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each location of the array. In an exemplary embodiment, background is calculated as the average hybridization signal intensity for the lowest 5% to 10% of the probes in the array. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all. Of course, one of skill in the art will appreciate that hybridization signals may be controlled for background using one or a combination of known approached, including one or a combination of approaches described in this paragraph.

The hybridization-based assay will be generally conducted under conditions in which the probe(s) will hybridize to their intended target subsequence, but with only insubstantial hybridization to other sequences or to other sequences, such that the difference may be identified. Such conditions are sometimes called “stringent conditions.” Stringent conditions are sequence-dependent and can vary under different circumstances. For example, longer probe sequences generally hybridize to perfectly complementary sequences (over less than fully complementary sequences) at higher temperatures. Generally, stringent conditions may be selected to be about 5 degrees C. lower than the thermal melting point (Tm) for the specific sequence at a defined ionic strength and pH. Exemplary stringent conditions may include those in which the salt concentration is at least about 0.01 to 1.0 M Na⁺ ion concentration (or other salts) at pH 7.0 to 8.3 and the temperature is at least about 30 degrees C. for short probes (e.g., 10 to 50 nucleotides). Desired hybridization conditions may also be achieved with the addition of agents such as formamide or tetramethyl ammonium chloride (TMAC).

When using an array, one of skill in the art will appreciate that a large number of array designs are suitable for the practice of this invention. The array will typically include a number of test probes that specifically hybridize to the sequences of interest. That is, the array will include probes designed to hybridize to any region of the BAD pathway genes contributing to the gene expression profile. In instances where the gene reference is an EST, probes may be designed from that sequence or from other regions of the corresponding full-length transcript that may be available in any of the public sequence databases, such as those herein described. See WO 99/32660 for methods of producing probes for a given gene or genes. In addition, software is commercially available for designing specific probe sequences. Typically, the array will also include one or more control probes, such as probes specific for a constitutively expressed gene, thereby allowing data from different hybridizations to be normalized or controlled.

The hybridization-based assays may include, in addition to “test probes” (e.g., that bind the target sequences of interest, which are BAD pathway gene sequences), the assay may also test for hybridization to one or a combination of control probes. Exemplary control probes include: normalization controls, expression level controls, and mismatch controls. For example, when determining the levels of gene expression in patient or control samples, the expression values may be normalized to control between samples. That is, the levels of gene expression in each sample may be normalized by determining the level of expression of at least one constitutively expressed gene in each sample. In accordance with the invention, the constitutively expressed gene is generally a transcript that is not differentially expressed in test samples (e.g., tumor samples).

Other useful controls are normalization controls, for example, using probes designed to be complementary to a labeled reference oligonucleotide added to the nucleic acid sample to be assayed. The signals obtained from the normalization controls after hybridization provide a control for variations in hybridization conditions, label intensity, “reading” efficiency and other factors that may cause the signal of a perfect hybridization to vary between arrays. In some embodiment, signals (e.g., fluorescence intensity) read from all other probes in the array are divided by the signal (e.g., fluorescence intensity) from the control probes thereby normalizing the measurements. Exemplary normalization probes are selected to reflect the average length of the other probes (e.g., test probes) present in the array, however, they may be selected to cover a range of lengths. The normalization control(s) may also be selected to reflect the (average) base composition of the other probes in the array. In some embodiments, the assay employs one or a few normalization probes, and they are selected such that they hybridize well (i.e., no secondary structure) and do not hybridize to any potential targets.

The hybridization-based assay may employ expression level controls, for example, probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes”.

The hybridization-based assay may also employ mismatch controls for the target sequences, and/or for expression level controls or for normalization controls. Mismatch controls are probes designed to be identical to their corresponding test or control probes, except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20-mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present, the perfect match probes should provide a more intense signal than the mismatch probes. The difference in intensity between the perfect match and the mismatch probe helps to provide a good measure of the concentration of the hybridized material.

Alternatively, the invention may employ reverse transcription polymerase chain reaction (RT-PCR), which is a sensitive method for the detection of mRNA, including low abundant mRNAs present in clinical samples. The application of fluorescence techniques to RT-PCR combined with suitable instrumentation has led to quantitative RT-PCR methods that combine amplification, detection and quantification in a closed system. Two commonly used quantitative RT-PCR techniques are the Taqman RT-PCR assay (ABI, Foster City, USA) and the Lightcycler assay (Roche, USA).

The design of appropriate probes for hybridizing to a particular target nucleic acid, and as configured for any appropriate nucleic acid detection assay, is well known.

### Computer Systems

In another aspect, the invention is a computer system that contains a database, on a computer-readable medium, of gene expression values indicative of a BAD pathway gene signature (BAD pathway score) and/or the BAD pathway score itself. These gene expression values and scores can be determined (as already described) in established cell lines, cell cultures established from patient samples, or directly from patient specimens, and for BAD pathway genes disclosed herein. The database may include, for each gene and/or score, chemo-sensitive and chemo-resistant gene expression levels, prognostic gene expression levels, thresholds, or Mean values, as well as various statistical measures, including measures of value dispersion (e.g., Standard Variation), fold change (e.g., between sensitive and resistant samples), and statistical significance (statistical association with drug sensitivity or resistance). Generally, signatures may be assembled based upon parameters to be selected and input by a user, with these parameters including of cancer or tumor type, histology, and/or candidate chemotherapeutic agents or combinations.

In certain embodiments, the database contains mean gene expression values for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 or more BAD pathway genes.

The computer system of the invention may be programmed to compare, score, or classify (e.g., in response to user inputs) a gene expression profile (preferably, a BAD pathway score) against a reference gene expression profile (preferably, a BAD pathway score) stored and/or generated from the database, to determine whether the gene expression profile of interest is itself a chemo-sensitive or chemo-resistant profile, or a profile consistent or inconsistent with an aspect of cancer prognosis such as survival or disease development or progression (e.g., metastasis, transition, tumor size progression, progression from chemo-sensitivity to chemo-resistance). For example, the computer system may be programmed to perform any of the known classification schemes for classifying gene expression profiles. Various classification schemes are known for classifying samples, and these include, without limitation: Principal Components Analysis (PCA), Naive Bayes, Support Vector Machines, Nearest Neighbors, Decision Trees, Logistic, Artificial Neural Networks, and Rule-based schemes. The computer system may employ a classification algorithm or “class predictor” as described in R. Simon, Diagnostic and prognostic prediction using gene expression profiles in high-dimensional microarray data, British Journal of Cancer 89:1599-1604 (2003), which is hereby incorporated by reference in its entirety.

The computer system of the invention may comprise a user interface, allowing a user to input gene expression values for comparison to a drug-sensitive and/or drug-resistant gene expression profile. The patient's gene expression values may be input from a location remote from the database.

The computer system may further comprise a display, for presenting and/or displaying a result, such as a BAD pathway signature assembled from the database, or the result of a comparison (or classification) between input gene expression values and one or more chemo-sensitive and/or chemo-resistant gene signatures, and/or one or more prognostic gene signatures. Such results may further be provided in any form (e.g., as a printable or printed report or other output).

The computer system of the invention may further comprise relational databases containing sequence information, for instance, for the BAD pathway genes. For example, the database may contain information associated with a given gene, cell line, or patient sample used for preparing BAD pathway gene signatures, such as descriptive information about the gene associated with the sequence information, or descriptive information concerning the clinical status of the patient (e.g., treatment regimen and outcome). The database may be designed to include different parts, for instance a sequence database and a gene expression database. Methods for the configuration and construction of such databases and computer-readable media to which such databases are saved are widely available, for instance, see U.S. Pat. No. 5,953,727, which is hereby incorporated by reference in its entirety.

The databases of the invention may be linked to an outside or external database (e.g., on the world wide web) such as GenBank (ncbi.nlm.nih.gov/entrez.index.html); KEGG (genome.ad.jp/kegg); SPAD (grt.kuyshu-u.ac.jp/spad/ind ex.html); HUGO (gene.ucl.ac.uk/hugo); Swiss-Prot (expasy.ch.sprot); Prosite (expasy.ch/tools/scnpsitl.html); OMIM (ncbi.nlm.nih.gov/omim); and GDB (gdb.org). In certain embodiments, the external database is GenBank and the associated databases maintained by the National Center for Biotechnology Information (NCBI) (ncbi.nlm.nih.gov).

Any appropriate computer platform, user interface, etc. may be used to perform the necessary comparisons between sequence information, gene expression information (e.g., BAD pathway gene expression profiles) and any other information in the database or information provided as an input. For example, a large number of computer workstations are available from a variety of manufacturers. Client/server environments, database servers and networks are also widely available and appropriate platforms for the databases described herein.

The databases of the invention may be used to produce, among other things, electronic Northerns that allow the user to determine the samples in which a given BAD pathway gene is expressed and to allow determination of the abundance or expression level of the given BAD pathway gene.

### Kits

The invention further provides a kit or probe array containing nucleic acid primers and/or probes for determining the level of expression in a sample (e.g., a patient tumor specimen or cell culture) of a plurality of BAD pathway genes. The probe array may contain 3000 probes or less, 2000 probes or less, 1000 probes or less, or 500 probes or less, or 400 or less probes, or 300 or less probes, or 200 or less probes so to embody a custom set for preparing BAD pathway gene expression profiles as described herein. In some embodiments, the kit may consist essentially of primers and/or probes related to evaluating chemo-sensitivity/resistance, and or prognosis, in a sample, and primers and/or probes related to necessary or meaningful assay controls (such as expression level controls and normalization controls). The kit for evaluating chemo-sensitivity/resistance and/or cancer prognosis may comprise nucleic acid probes and/or primers designed to detect the BAD pathway gene expression level of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 or more BAD pathway genes.

The kit may include a set of probes and/or primers designed to detect or quantify the expression levels of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 or more BAD pathway genes. The primers and/or probes may be designed to detect BAD pathway gene expression levels in accordance with any assay format, including those described herein. Exemplary assay formats include polymerase-based assays, such as RT-PCR, Taqman™, hybridization-based assays, for example using DNA microarrays or other solid support, nucleic acid sequence based amplification (NASBA), flap endonuclease-based assays. The kit may, but need not employ a DNA microarray or other high density detection format.

In accordance with this aspect, the probes and primers may comprise antisense nucleic acids or oligonucleotides that are wholly or partially complementary to the nucleic acid targets described herein (e.g., BAD pathway genes). The probes and primers will be designed to detect the particular target via an available nucleic acid detection assay format, which are well known in the art. The kits of the invention may comprise probes and/or primers designed to detect the diagnostic targets via detection methods that include amplification, endonuclease cleavage, and hybridization.

The methods of the present invention can be used with humans and other animal subjects of any gender. The terms “subject”, “patient”, and “individual” are used interchangeably to refer to human and non-human animals. The non-human animals contemplated within the scope of the invention include mammalian and non-mammalian animals, including domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses.

As used herein, the term “level” refers to the amount, accumulation, or rate of a biomarker molecule. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a molecule accumulated in a cell, including, for example, the amount of particular post-synthetic modifications of a molecule such as a polypeptide, nucleic acid or small molecule. The term can be used to refer to an absolute amount of a molecule in a sample or to a relative amount of the molecule, including amounts determined under steady-state or non-steady-state conditions. The expression level of a molecule can be determined relative to a control component molecule in a sample.

A gene expression level of a molecule is intended to mean the amount, accumulation, or rate of synthesis of a biomarker gene. The gene expression level can be represented by, for example, the amount or transcription rate of hnRNA or mRNA encoded by a gene. A gene expression level similarly refers to an absolute or relative amount or a synthesis rate determined, for example, under steady-state or non-steady-state conditions.

As used herein, the term “sample” is intended to mean any biological fluid, cell, tissue, organ, or portion of any of the foregoing, that includes or potentially includes genetic material for a gene expression signature. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid molecule or polypeptide preparation.

Biological samples refer to a composition obtained from a human or animal subject. Biological samples within the scope of the invention include, but are not limited to, cancer cells (e.g., tumor cells), ascites fluid, whole blood, blood plasma, serum, urine, tears, saliva, sputum, exhaled breath, nasal secretions, pharyngeal exudates, bronchioalveolar lavage, tracheal aspirations, interstitial fluid, lymph fluid, meningeal fluid, amniotic fluid, glandular fluid, feces, perspiration, mucous, vaginal or urethral secretion, cerebrospinal fluid, and transdermal exudate. A biological sample also includes experimentally separated fractions of all of the preceding solutions or mixtures containing homogenized solid material, such as feces, tissues, and biopsy samples.

Samples and/or binding moieties may be arrayed on a solid support, or multiple supports can be utilized, for multiplex detection or analysis. “Arraying” refers to the act of organizing or arranging members of a library (e.g., an array of different samples), or other collection, into a logical or physical array. Thus, an “array” refers to a physical or logical arrangement of, e.g., biological samples. A physical array can be any “spatial format” or physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding moieties can be plated or otherwise deposited in microtitered, e.g., 96-well, 384-well, or-1536 well, plates (or trays). Optionally, binding moieties may be immobilized on the solid support.

As used herein, the terms “array” and “microarray” are interchangeable and can include an arrangement of a collection of nucleotide sequences in a centralized location. Arrays can be on a solid substrate, such as a glass slide, or on a semi-solid substrate, such as nitrocellulose membrane. The nucleotide sequences can be DNA, RNA, or any permutations thereof. The nucleotide sequences can also be partial sequences from a gene, primers, whole gene sequences, non-coding sequences, coding sequences, published sequences, known sequences, or novel sequences.

Detection of cancer biomarkers, and other assays that are to be carried out on samples, can be carried out simultaneously or sequentially, and may be carried out in an automated fashion, in a high-throughput format.

“Platinum-based therapy” and “platinum-based chemotherapy” are used interchangeably herein and refers to agents or compounds that are associated with platinum.

A “complete response” (CR) is defined as a complete disappearance of all measurable and assessable disease or, in the absence of measurable lesions. An individual who exhibits a complete response is known as a “complete responder.”

An “incomplete response” (IR) includes those who exhibited a “partial response” (PR), had “stable disease” (SD), or demonstrated “progressive disease” (PD) during primary therapy.

The terms “treatment”, “treating” and the like are intended to mean obtaining a desired pharmacologic and/or physiologic effect, e.g., slowing or stopping cancer progression, time to relapse, or alleviating one or more symptoms of a disorder such as cancer. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease (for example, cancer) in a mammal, particularly a human, and includes: (a) preventing a disease or condition (e.g., preventing cancer) from occurring or recurring in an individual who may be predisposed to the disease but has not yet been diagnosed as having it; (b) inhibiting the disease, (e.g., arresting its development); or (c) relieving the disease (e.g., reducing symptoms associated with the disease). In some embodiments, the subject is suffering from the disorder (e.g., cancer), and treatment includes identifying the subject as suffering from the disorder (e.g., cancer) prior to administration of an effective amount of an agent such as a chemotherapeutic agent or an agent that targets the BAD pathway.

An “effective amount” refers to an amount of an agent, such as a chemotherapeutic agent or an agent that targets the BAD pathway, that is sufficient to exert a biological effect in the individual. In some embodiments, an effective amount is an amount sufficient to exert cytotoxic effects on cancerous cells. In some embodiments, an effective amount is an amount sufficient to alleviate at least one symptom of a cancer or malignancy.

“Predicting” and “prediction” as used herein does not necessarily mean that the event will happen with 100% certainty; rather, it is intended to mean the event will more likely occur than not occur.

As used herein, the term “disease free survival” refers to the lack of detectable disease recurrence and the fate of a patient after diagnosis, i.e., a patient who is alive without disease recurrence. For example, if the patient has prostate cancer, disease recurrence would be recurrence of a prostate tumor or metastasis from such a tumor. The phrase “overall survival” refers to the fate of the patient after diagnosis, regardless of whether the patient has a recurrence of the disease.

As used in the context of a course of treatment, “effectiveness” refers to the ability of the course of treatment to decrease the risk of disease recurrence or spread and therefore to increase the likelihood of disease-free or overall survival of the patient. This method will have particular utility when the gene expression level in a sample of a patient is abnormal compared to a reference gene expression level. Comparison of gene expression levels in a sample from a patient before and after treatment can thereby serve to indicate whether a gene expression level is returning to a normal or healthy level, implying a more effective course of treatment, or whether a gene expression level is remaining abnormal or increasing in abnormality, implying a less effective course of treatment.

As used herein, the terms solid “support”, “substrate”, and “surface” refer to a solid phase which is a porous or non-porous water insoluble material that can have any of a number of shapes, such as strip, rod, particle, beads, or multi-welled plate. In some embodiments, the support has a fixed organizational support matrix that preferably functions as an organization matrix, such as a microtiter tray. Solid support materials include, but are not limited to, cellulose, polysaccharide such as Sephadex, glass, polyacryloylmorpholide, silica, controlled pore glass (CPG), polystyrene, polystyrene/latex, polyethylene such as ultra high molecular weight polyethylene (UPE), polyamide, polyvinylidine fluoride (PVDF), polytetrafluoroethylene (PTFE; TEFLON), carboxyl modified teflon, nylon, nitrocellulose, and metals and alloys such as gold, platinum and palladium. The solid support can be biological, non-biological, organic, inorganic, or a combination of any of these, existing as particles, strands, precipitates, gels, sheets, pads, cards, strips, dipsticks, test strips, tubing, spheres, containers, capillaries, pads, slices, films, plates, slides, etc., depending upon the particular application. Preferably, the solid support is planar in shape, to facilitate contact with a biological sample such as urine, whole blood, plasma, serum, peritoneal fluid, or ascites fluid. Other suitable solid support materials will be readily apparent to those of skill in the art. The solid support can be a membrane, with or without a backing (e.g., polystyrene or polyester card backing), such as those available from Millipore Corp. (Bedford, Mass.), e.g., HI-FLOW Plus membrane cards. The surface of the solid support may contain reactive groups, such as carboxyl, amino, hydroxyl, thiol, or the like for the attachment of nucleic acids, proteins, etc. Surfaces on the solid support will sometimes, though not always, be composed of the same material as the support. Thus, the surface can be composed of any of a wide variety of materials, such as polymers, plastics, resins, polysaccharides, silica or silica-based materials, carbon, metals, inorganic glasses, membranes, or any of the aforementioned support materials (e.g., as a layer or coating).

As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, quantum dots, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group.

As used in this specification, the singular forms “a”, “an”, and “the” include plural reference unless the context clearly dictates otherwise. Thus, for example, a reference to “a gene” includes more than one such gene. A reference to “a compound” includes more than one such compound, and so forth.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### BAD Pathway Gene Signature as Determinant of Development and Progression of Cancer, and Patient Survival

Accurate methods are needed to predict survival and treatment response in individual cancer patients. The inventors have developed a genetic signature score associated with survival in patients with certain cancers. This score is a useful prognostic tool as well as an opportunity for targeted therapy.

The BAD pathway score is based on expression of certain genes in the BAD pathway (Bcl-2 Associated Death promoter). This pathway may regulate phosphorylation of the BAD protein, and in turn influence how susceptible cancer cells are to therapy, and how likely they are to self-destruct. The BAD pathway score is higher in cancer samples than in pre-cancer samples or normal tissue, suggesting that the BAD pathway is important in cancer development and progression as well as clinical outcome.

The BAD pathway score was tested on more than 1,700 patient samples from ovarian, endometrial, colon, lung, breast and brain tumors. The score is most highly associated with survival in ovarian, breast, colon and brain cancers; and with development and progression of breast and endometrial cancers. The score is based on gene expression analysis of 53 genes, or a subset thereof, and has been validated with external data sets. The score of a patient's sample can be compared to a reference profile represented by a reference score, such as a cut-off value, indicating either long or short survival time. The BAD pathway score can be used to determine whether aggressive therapy is be needed, or that chemotherapy targeting the BAD pathway may be efficacious. Evidence from ovarian cancer suggests the score is more accurate than de-bulking outcome in predicting survival.

### Materials and Methods

The inventors have identified 53 genes (Table 1) in the BAD apoptosis pathway by using genomic data previously generated by their group (from a series of ovarian cancer cell line cisplatin treatments, paralleled by measures of cisplatin resistance and global gene expression). These genes were evaluated as a “BAD-pathway gene expression signature”.

TABLE 1
BAD Pathway Signature Genes

| U133Plus: | Gene name | probe set ID | u133A: | probe set ID | gene names |
|---|---|---|---|---|---|
| 1 | MAP2K2 | 202424_at | 1 | 202424_at | MAP2K2 |
| 2 | MAP2K2 | 213487_at | 2 | 213487_at | MAP2K2 |
| 3 | MAP2K2 | 213490_s_at | 3 | 213490_s_at | MAP2K2 |
| 4 | RAF1 | 1557675_at | 4 | 201244_s_at | RAF1 |
| 5 | RAF1 | 201244_s_at | 5 | 212983_at | HRAS |
| 6 | HRAS | 212983_at | 6 | 201469_ s _at | SHC1 |
| 7 | SHC1 | 201469_s_at | 7 | 214853_s_at | SHC1 |
| 8 | SHC1 | 214853_s_at | 8 | 217048_at | — |
| 9 | SHC1 | 217048_at | 9 | 201983_s_at | EGFR |
| 10 | EGFR | 1565483_at | 10 | 201984_s_at | EGFR |
| 11 | EGFR | 1565484_x_at | 11 | 210984_x_at | EGFR |
| 12 | EGFR | 201983_s_at | 12 | 211550_at | EGFR |
| 13 | EGFR | 201984_s_at | 13 | 211551_at | EGFR |
| 14 | EGFR | 210984_x_at | 14 | 211607_x_at | EGFR |
| 15 | EGFR | 211550_at | 15 | 204686_at | IRS1 |
| 16 | EGFR | 211551_at | 16 | 204369_at | PIK3CA |
| 17 | EGFR | 211607_x_at | 17 | 212688_at | PIK3CB |
| 18 | IRS1 | 204686_at | 18 | 217620_s_at | PIK3CB |
| 19 | IRS1 | 238933_at | 19 | 203879_at | PIK3CD |
| 20 | PIK3CA | 204369_at | 20 | 211230_s_at | PIK3CD |
| 21 | PIK3CA | 231854_at | 21 | 203379_at | RPS6KA1 |
| 22 | PIK3CA | 235980_at | 22 | 204906_at | RPS6KA2 |
| 23 | PIK3CB | 212688_at | 23 | 212912_at | RPS6KA2 |
| 24 | PIK3CB | 217620_s_at | 24 | 203843_at | RPS6KA3 |
| 25 | PIK3CD | 203879_at | 25 | 1861 _at | BAD |
| 26 | PIK3CD | 211230_s_at | 26 | 209364_at | BAD |
| 27 | RPS6KA1 | 203379_at | 27 | 206665_s_at | BCL2L1 |
| 28 | RPS6KA2 | 1557970_s_at | 28 | 212312_at | BCL2L1 |
| 29 | RPS6KA2 | 204906_at | 29 | 215037_s_at | BCL2L1 |
| 30 | RPS6KA2 | 212912_at | 30 | 208478_s_at | BAX |
| 31 | RPS6KA3 | 203843_at | 31 | 211833_s_at | BAX |
| 32 | RPS6KA3 | 226335_at | 32 | 207163_s_at | AKT1 |
| 33 | BAD | 1861_at | 33 | 203808_at | — |
| 34 | BAD | 209364_at | 34 | 203809_s_at | AKT2 |
| 35 | BAD | 232660_at | 35 | 211453_s_at | AKT2 |
| 36 | BCL2L1 | 206665_s_at | 36 | 212607_at | AKT3 |
| 37 | BCL2L1 | 212312_at | 37 | 212609_s_at | AKT3 |
| 38 | BCL2L1 | 215037_s_at | 38 | 219393_s_at | AKT3 |
| 39 | BCL2L1 | 231228_at | 39 | 200744_s_at | GNB1 |
| 40 | BAX | 208478_s_at | 40 | 200745_s_at | GNB1 |
| 41 | BAX | 211833_s_at | 41 | 200746_s_at | GNB1 |
| 42 | AKT1 | 207163_s_at | 42 | 200852_x_at | GNB2 |
| 43 | AKT2 | 1560689_s_at | 43 | 217450_at | — |
| 44 | AKT2 | 203808_at | 44 | 206047_at | GNB3 |
| 45 | AKT2 | 203809_s_at | 45 | 204000_at | GNB5 |
| 46 | AKT2 | 211453_s_at | 46 | 207124_s_at | GNB5 |
| 47 | AKT2 | 225471_s_at | 47 | 211871_x_at | GNB5 |
| 48 | AKT2 | 226156_at | 48 | 201921_at | GNG10///LOC552891 |
| 49 | AKT2 | 236664_at | 49 | 204115_at | GNG11 |
| 50 | AKT3 | 212607_at | 50 | 212294_at | GNG12 |
| 51 | AKT3 | 212609_s_at | 51 | 220806_x_at | GNG13 |
| 52 | AKT3 | 219393_s_at | 52 | 222005_s_at | GNG3 |
| 53 | AKT3 | 222880_at | 53 | 205184 _at | GNG4 |
| 54 | AKT3 | 224229_s_at | 54 | 207157_s_at | GNG5 |
| 55 | AKT3 | 242876_at | 55 | 206896_s_at | GNG7 |
| 56 | GNB1 | 1570108_at | 56 | 214227 _at | GNG7 |
| 57 | GNB1 | 200744_s_at | 57 | 217327_ at | — |
| 58 | GNB1 | 200745_s_at | 58 | 207166_at | GNGT1 |
| 59 | GNB1 | 200746_s_at | 59 | 203966_s_at | PPM1A |
| 60 | GNB2 | 200852_x_at | 60 | 210407_at | PPM1A |
| 61 | GNB2 | 217450_at | 61 | 209296_ at | PPM1B |
| 62 | GN B3 | 206047_at | 62 | 213225_at | PPM1B |
| 63 | GNB4 | 223487_x_at | 63 | 204566_at | PPM1D |
| 64 | GNB4 | 223488_s_at | 64 | 203063_at | PPM1F |
| 65 | GNB4 | 225710_at | 65 | 37384_at | PPM1F |
| 66 | GNB5 | 1554346_at | 66 | 200913_at | PPM1G |
| 67 | GNB5 | 204000_at | 67 | 218273_s_at | PPM2C |
| 68 | GNB5 | 207124_s_at | 68 | 205867_at | PTPN11 |
| 69 | GNB5 | 211871_x_at | 69 | 205868_s_at | PTPN11 |
| 70 | GNG10 | 201921_at | 70 | 209895_ at | PTPN11 |
| 71 | GNG11 | 204115_at | 71 | 209896_s_at | PTPN11 |
| 72 | GNG12 | 1555240_s_at | 72 | 212610_at | PTPN11 |
| 73 | GNG12 | 212294_at | 73 | 200780_x_at | GNAS |
| 74 | GNG12 | 222834_s_at | 74 | 200981_x_at | GNAS |
| 75 | GNG13 | 220806_x_at | 75 | 211858_x_at | GNAS |
| 76 | GNG2 | 1555766_a_at | 76 | 212273_x_at | GNAS |
| 77 | GNG2 | 223943_s_at | 77 | 214157_at | GNAS |
| 78 | GNG2 | 224964_s_at | 78 | 214548_x_at | GNAS |
| 79 | GNG2 | 224965_at | 79 | 217057_s_at | GNAS |
| 80 | GNG3 | 222005_s_at | 80 | 217058 _at | GNAS |
| 81 | GNB4 | 223487_x_at | 81 | 217673_x_at | GNAS |
| 82 | GNB4 | 223488_s_at | 82 | 200603_at | PRKAR1A |
| 83 | GNB4 | 225710_at | 83 | 200604_s_at | PRKAR1A |
| 84 | GNB5 | 1554346_at | 84 | 200605_s_at | PRKAR1A |
| 85 | GNB5 | 204000_at | 85 | 212555_at | PRKAR1B |
| 86 | GNB5 | 207124_s_at | 86 | 212559 _at | PRKAR1B |
| 87 | GNB5 | 211871_x_at | 87 | 204842_x_at | PRKAR2A |
| 88 | GNG10 | 201921_at | 88 | 204843_s_at | PRKAR2A |
| 89 | GNG11 | 204115_at | 89 | 213052_at | PRKAR2A |
| 90 | GNG12 | 1555240_s_at | 90 | 203680_at | PRKAR2B |
| 91 | GNG12 | 212294_at | 91 | 203213_at | CDC2 |
| 92 | GNG12 | 222834_s_at | 92 | 203214_x_at | CDC2 |
| 93 | GNG13 | 220806_x_at | 93 | 210559_s_at | CDC2 |
| 94 | GNG2 | 1555766_a_at | 94 | 202801_at | PRKACA |
| 95 | GNG2 | 223943_s_at | 95 | 216234_s_at | LOC730418///PRKACA |
| 96 | GNG2 | 224964_s_at | 96 | 202741_at | PRKACB |
| 97 | GNG2 | 224965_at | 97 | 202742_s_at | PRKACB |
| 98 | GNG3 | 222005_s_at | 98 | 207228_at | PRKACG |
| 99 | GNG4 | 1555765_a_at | | | |
| 100 | GNG4 | 1555867_at | | | |
| 101 | GNG4 | 1566513_a_at | | | |
| 102 | GNG4 | 205184_at | | | |
| 103 | GN G5 | 207157_s_at | | | |
| 104 | GNG7 | 206896_s_at | | | |
| 105 | GNG7 | 214227_at | | | |
| 106 | GNG7 | 217327_at | | | |
| 107 | GNG7 | 228831_s_at | | | |
| 108 | GNG7 | 232043_at | | | |
| 109 | GNG8 | 234284_at | | | |
| 110 | GNGT1 | 207166_at | | | |
| 111 | GNGT2 | 235139_at | | | |
| 112 | PPM1A | 203966_s_at | | | |
| 113 | PPM1A | 210407_at | | | |
| 114 | PPM1A | 231370_at | | | |
| 115 | PPM1B | 209296_at | | | |
| 116 | PPM1B | 213225_at | | | |
| 117 | PPM1D | 204566_at | | | |
| 118 | PPM1F | 1555091_at | | | |
| 119 | PPM1F | 1555470_a_at | | | |
| 120 | PPM1F | 203063_at | | | |
| 121 | PPM1F | 37384_at | | | |
| 122 | PPM1G | 200913_at | | | |
| 123 | PPM1L | 239855_at | | | |
| 124 | PPM2C | 218273_s_at | | | |
| 125 | PPM2C | 222572_at | | | |
| 126 | PPTC7 | 225204_at | | | |
| 127 | PPTC7 | 225213_at | | | |
| 128 | PPTC7 | 235744_at | | | |
| 129 | PTPN11 | 1552637_at | | | |
| 130 | PTPN11 | 205867_at | | | |
| 131 | PTPN11 | 205868_s_at | | | |
| 132 | PTPN11 | 209895_at | | | |
| 133 | PTPN11 | 209896_s_at | | | |
| 134 | PTPN11 | 212610_at | | | |
| 135 | GNAS | 200780_x_at | | | |
| 136 | GNAS | 200981_x_at | | | |
| 137 | GNAS | 211858_x_at | | | |
| 138 | GNAS | 212273_x_at | | | |
| 139 | GNAS | 214157_at | | | |
| 140 | GNAS | 214548_x_at | | | |
| 141 | GNAS | 217057_s_at | | | |
| 142 | GNAS | 217058_at | | | |
| 143 | GNAS | 217673_x_at | | | |
| 144 | GNAS | 228173_at | | | |
| 145 | GNAS | 229274_at | | | |
| 146 | GNAS | 235851_s_at | | | |
| 147 | GNAS | 239037_at | | | |
| 148 | PRKAR1A | 200603_at | | | |
| 149 | PRKAR1A | 200604_s_at | | | |
| 150 | PRKAR1A | 200605_s_at | | | |
| 151 | PRKAR1A | 242482_at | | | |
| 152 | PRKAR1B | 212555_at | | | |
| 153 | PRKAR1B | 212559_at | | | |
| 154 | PRKAR2A | 204842_x_at | | | |
| 155 | PRKAR2A | 204843_s_at | | | |
| 156 | PRKAR2A | 213052_at | | | |
| 157 | PRKAR2A | 225000_at | | | |
| 158 | PRKAR2A | 225011_at | | | |
| 159 | PRKAR2B | 203680_at | | | |
| 160 | CDC2 | 203213_at | | | |
| 161 | CDC2 | 203214_x_at | | | |
| 162 | CDC2 | 210559_s_at | | | |
| 163 | CDC2 | 231534_at | | | |
| 164 | PRKACA | 202801_at | | | |
| 165 | PRKACA | 216234_s_at | | | |
| 166 | PRKACB | 202741_at | | | |
| 167 | PRKACB | 202742_s_at | | | |
| 168 | PRKACB | 235780_at | | | |
| 169 | PRKACG | 207228_at | | | |

These 53 genes are represented by up to 169 Affymetrix U133Plus2 probe sets (47 genes and 98 probe sets for Affymetrix U133A genechips and 43 genes and 72 probe sets for Affymetrix U95A genechips). Using principal components analysis (PCA) method, the inventors derived a “pathway score” to represent an overall gene expression level for the 53 BAD pathway genes (or subsets thereof for data sets generated by U133A or U95A). Specifically, the inventors performed principal components analysis to reduce data dimension into a small set of uncorrelated principal components. This set of principal components was generated based on its ability to account for variation.

The inventors used the first principal component (1st PCA), as it accounts for the largest variability in the data, as a pathway score to represent the overall expression level for the BAD pathway. That is, pathway score=Σwᵢxᵢ, a weighted average expression among the BAD pathway genes, where xᵢ represents gene i expression level, wᵢ is the corresponding weight (loading coefficient) with Σwᵢ²=1, and the wᵢ values maximize the variance of Σwᵢxᵢ. This approach has been used to derive a malignancy pathway gene signature in a breast cancer study (Chen et. al., 2009).

The influence of this BAD pathway expression signature (pathway score) on patient survival was then evaluated in additional Affymetrix expression array datasets including:

i) 143 patients with ovarian cancer treated at Moffitt Cancer Center and Duke University Medical Center (The Moffitt/Duke OVCA dataset);

ii) GSE9891: 240 patients with ovarian cancer treated in Australia (The Australian OVCA dataset, Tothill et al, 2008);

iii) GSE2034: 286 patients with breast cancer, (The breast cancer 286 dataset, Carroll et al, 2006);

iv) 50 patients with Glioblastoma (Glioblastoma 50 set, Nutt et al, 2003);

v) 182 patients with Glioblastoma. (Glioblastoma 182 set, Lee et al, 2008);

vi) GSE4573: Lung 130 (Raponi et al, 2006);

vii) 205 patients with colon cancer treated at Moffitt Cancer Center (The MCC colon dataset);

viii) 33 endometrial samples; Boren et al. August; 110(2):206-15. Gynecol Oncol. 2008 (The MCC endometrial dataset);

ix) 61 breast samples (atypical ductal hyperplasia, ductal carcinoma in situ, and invasive ductal carcinoma) (The Ma et al. dataset);

x) 197 breast samples (normal, ductal carcinoma in situ, and invasive ductal carcinoma) (The MCC 197 breast dataset);

xi) Chanrion Tamoxifen-Treated Primary Breast Cancer Study (Relapse-Free vs. Relapse) (Charion dataset).

Specifically, BAD pathway analysis was performed in two ways. In the first way, association of BAD pathway with overall survival or relapse free survival was determined. For each dataset, expression data are first standardized (i.e., centered at mean and divided by standard deviation) and then PCA is implemented to obtain BAD pathway score for each subject. The median of the BAD pathway score was used as the cutoff to form two groups: high-level of BAD pathway group (>median) and low-level of BAD pathway group (<median). KM survival curves are generated and log-rank test is used to test any significant difference of survival curves. Results show significant association in the Moffitt/Duke OVCA dataset, the Australian OVCA dataset (GSE9891; n=240), and Wang's breast cancer relapse free survival (GSE2034; n=286).

In the second way, prediction of BAD pathway was determined by using the Moffitt/Duke OVCA dataset as the training set and the others as the test sets. For the Moffitt/Duke OVCA dataset, PCA is implemented to obtain the weight (loading coefficient) of each BAD pathway gene. The weights (derived from the Moffitt/Duke OVCA dataset) are then used to calculate BAD pathway score for each test dataset. The median of the BAD pathway score was used as the cutoff to form two groups: high-level of BAD pathway group (>median) and low-level of BAD pathway group (<median). KM survival curves are generated and log-rank test is used to test any significant difference of survival curves. Results do not show any significant evidence of prediction.

### Results

A. The Moffitt/Duke Ovarian Cancer (n=143) Dataset:

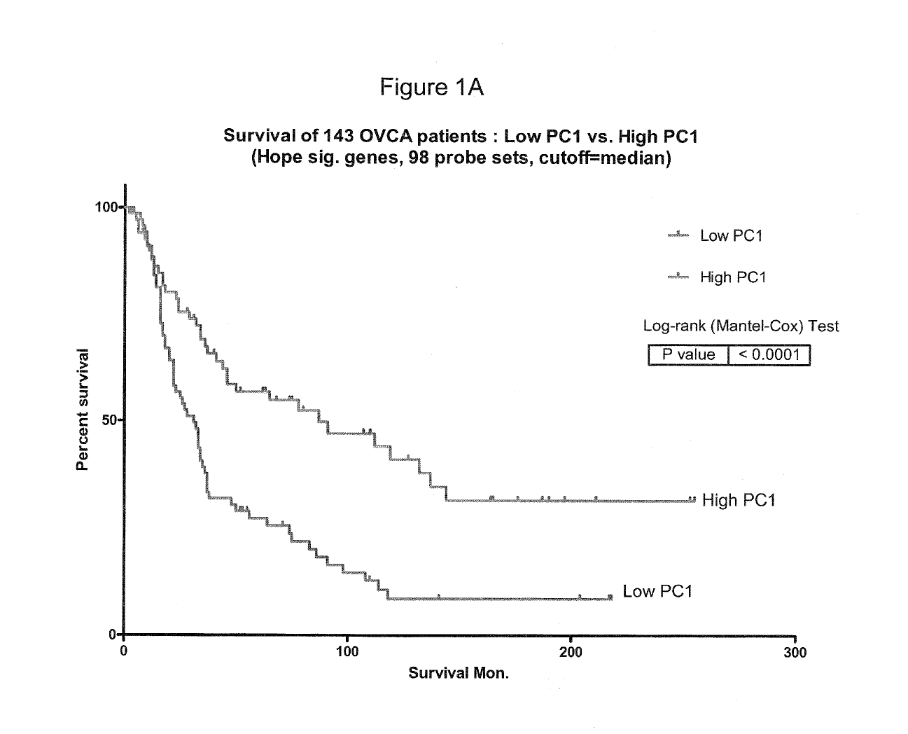

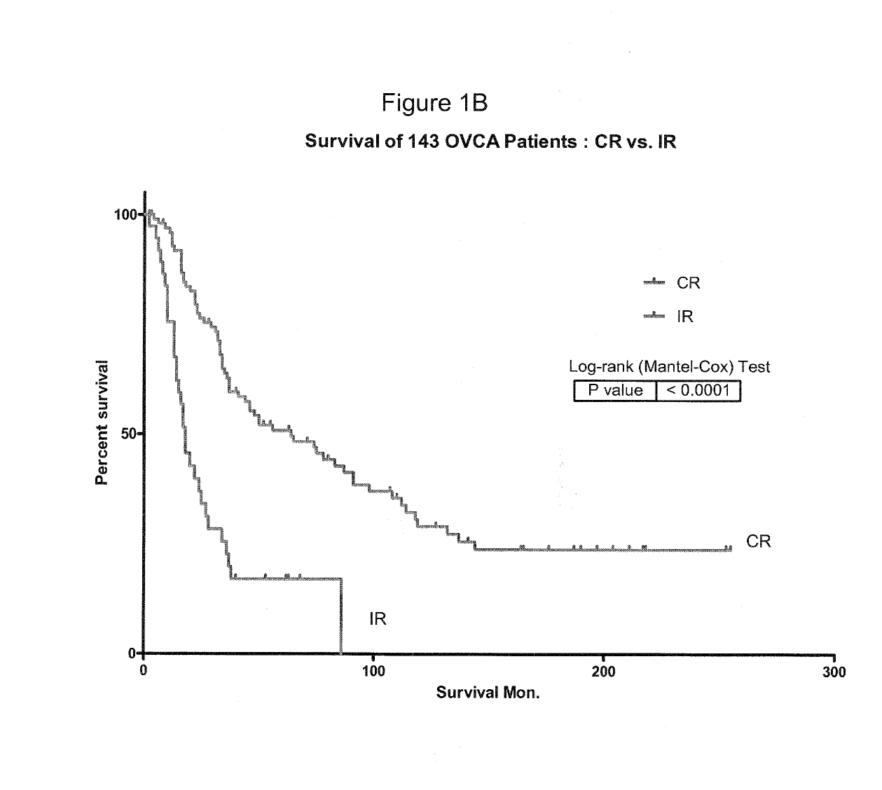

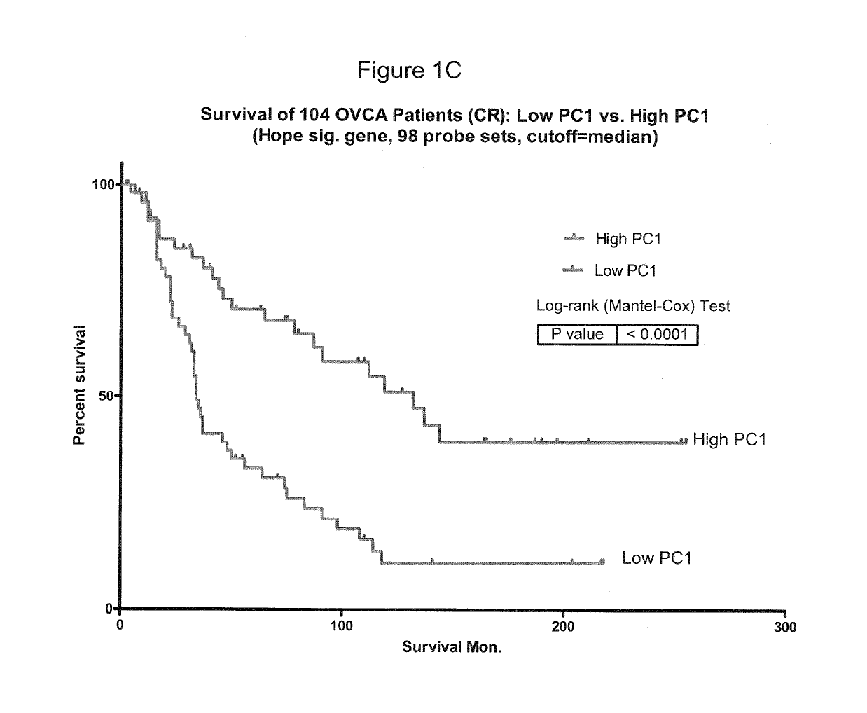

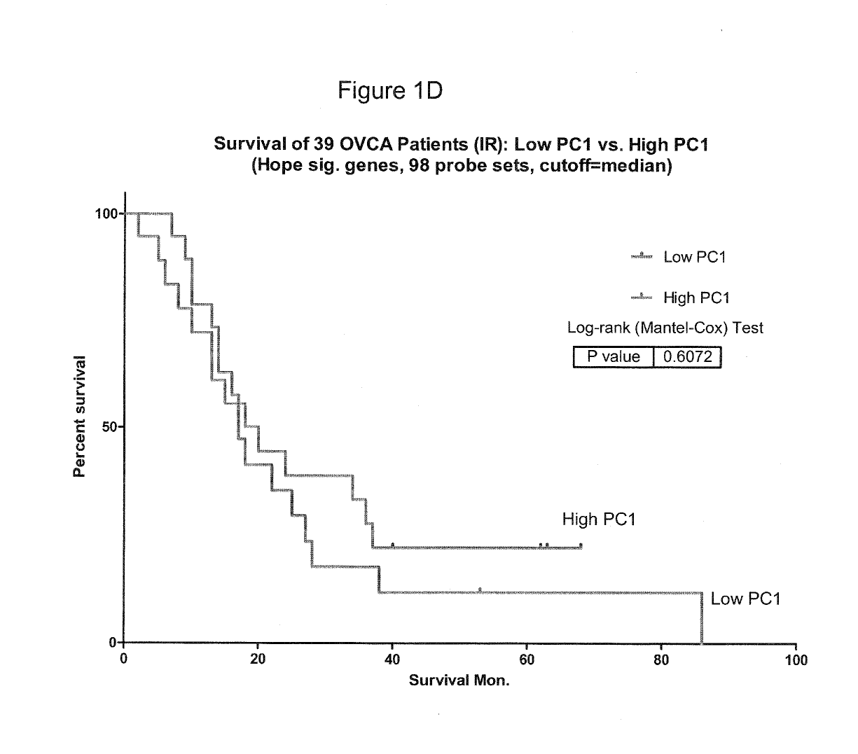

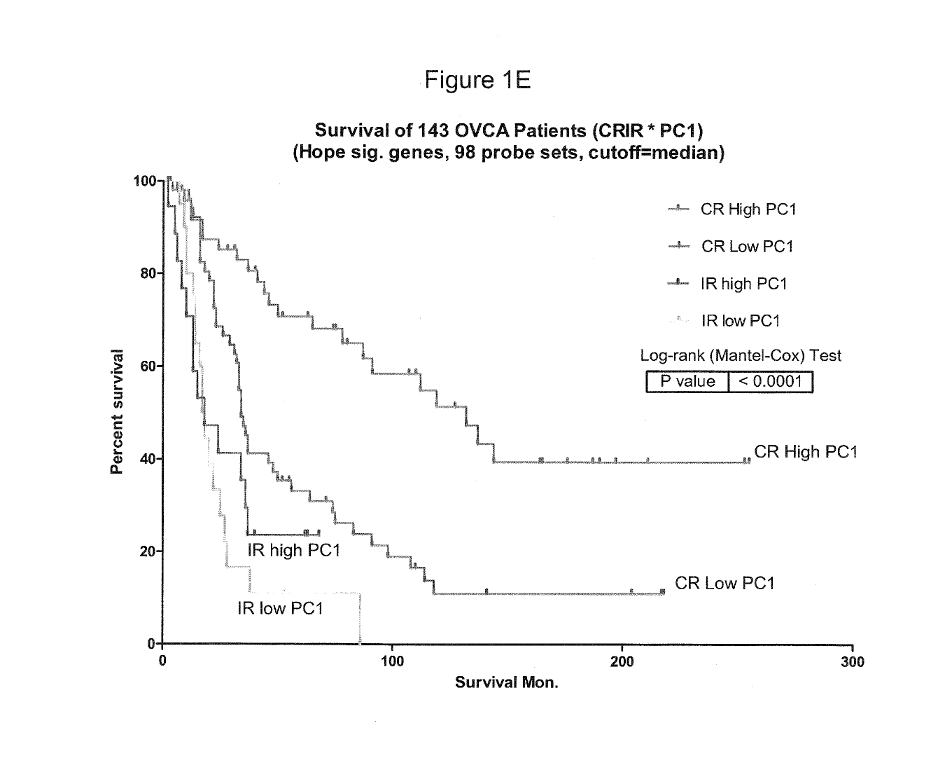

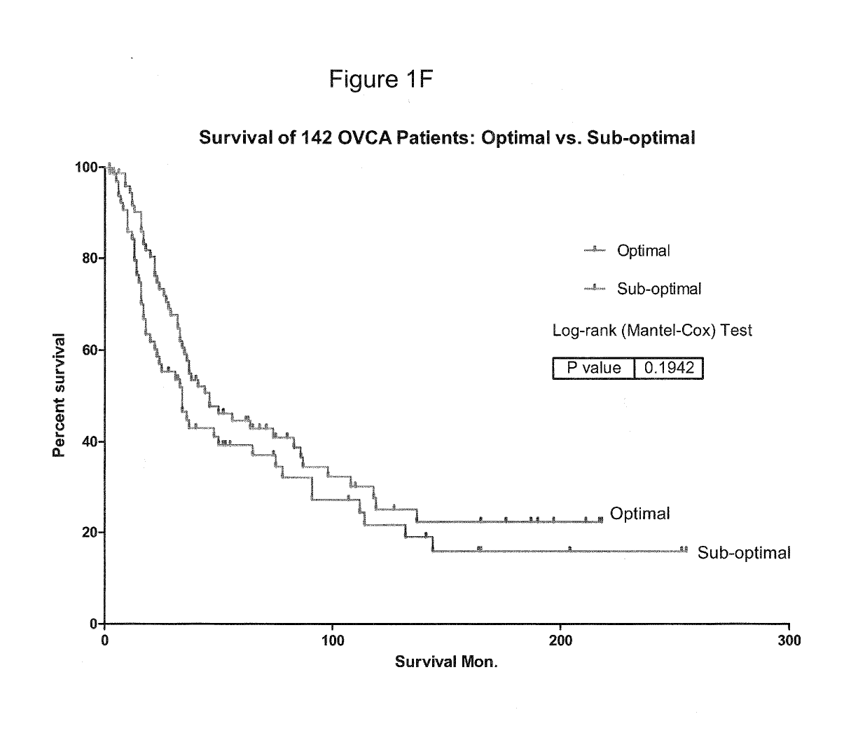

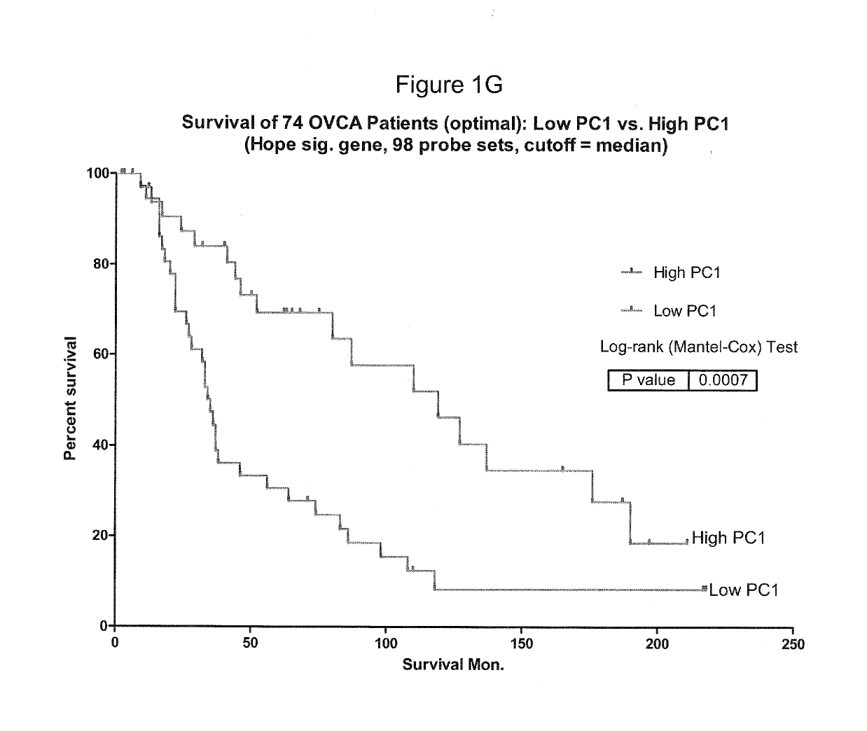

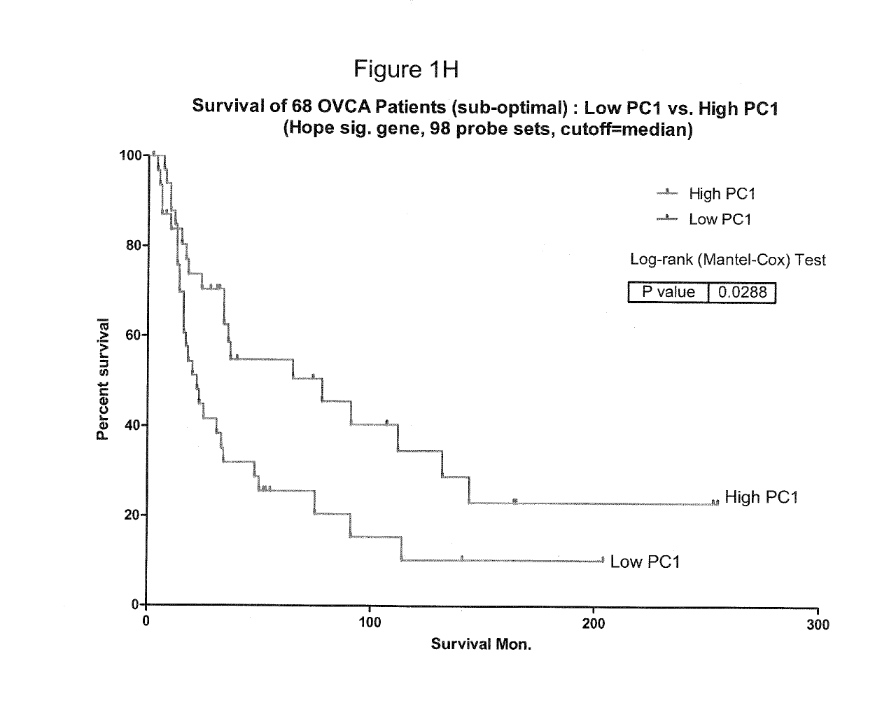

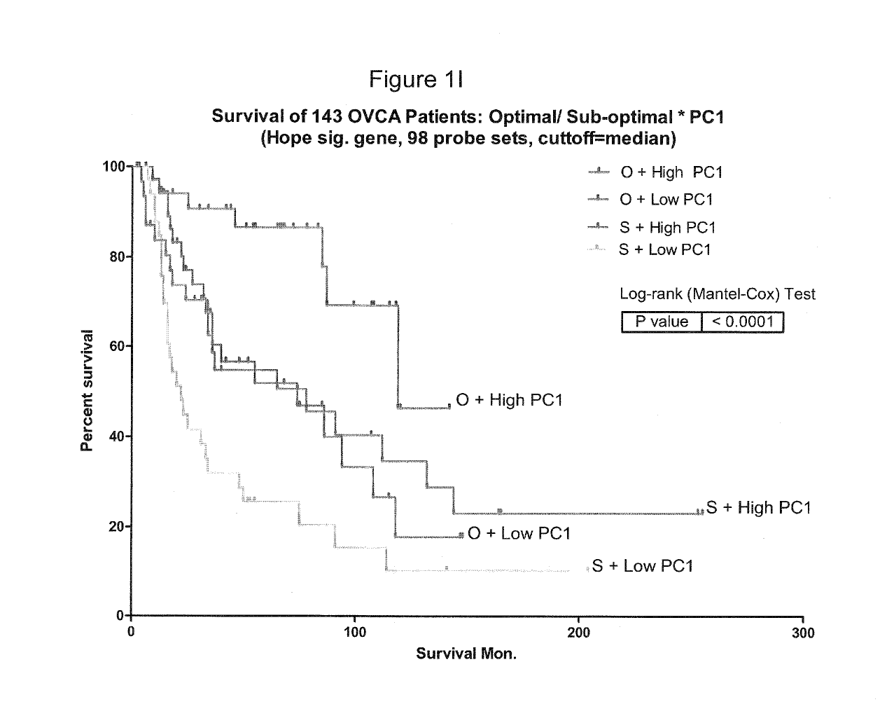

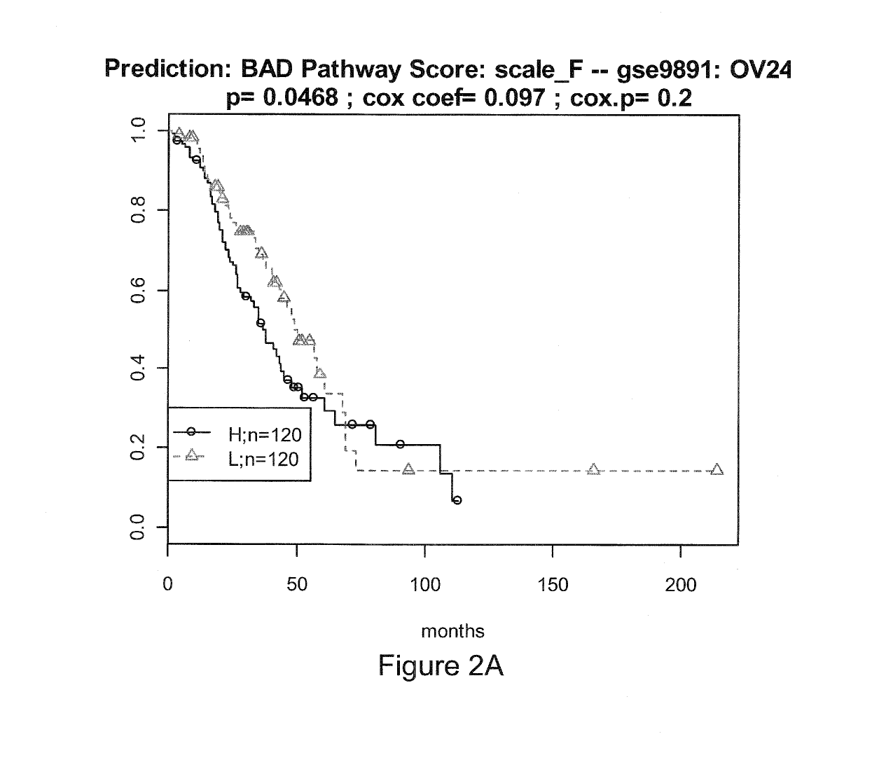

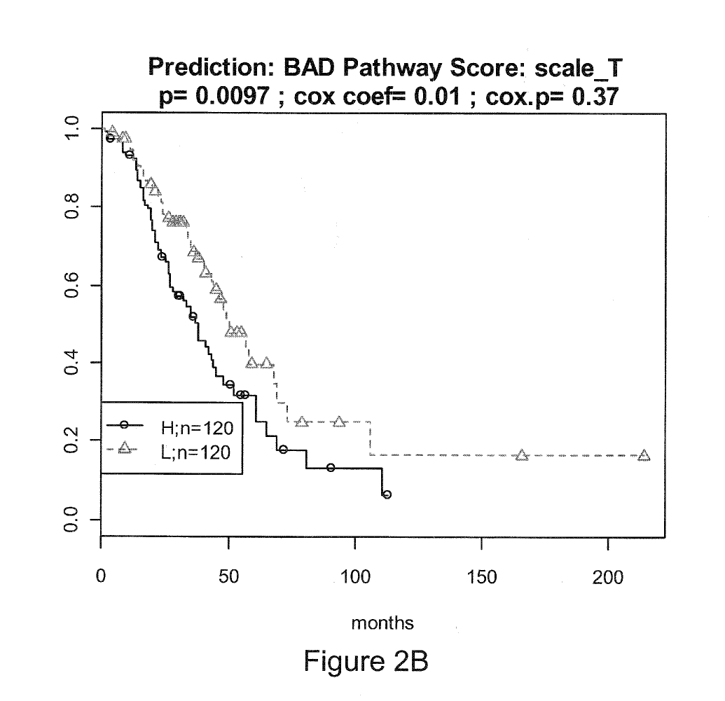

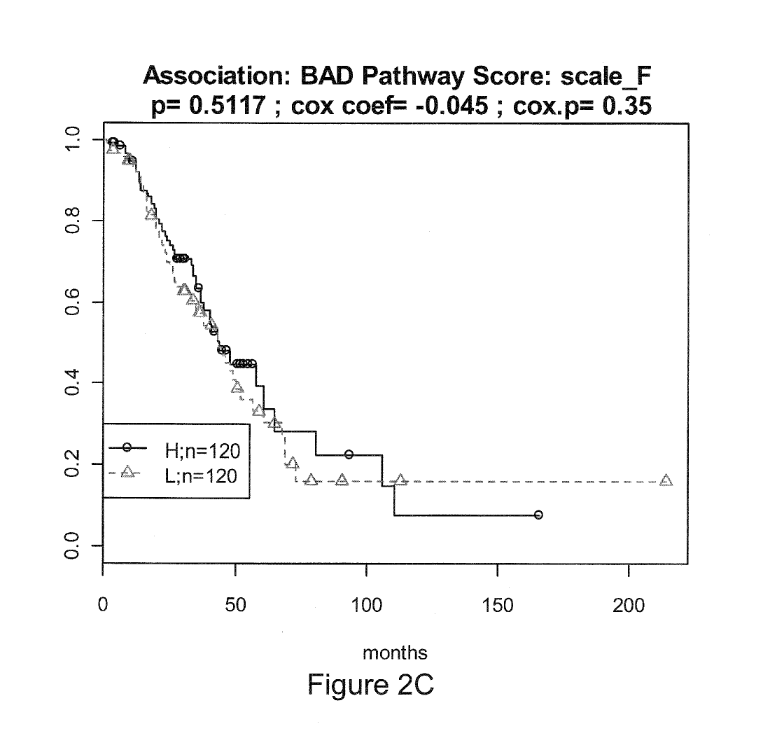

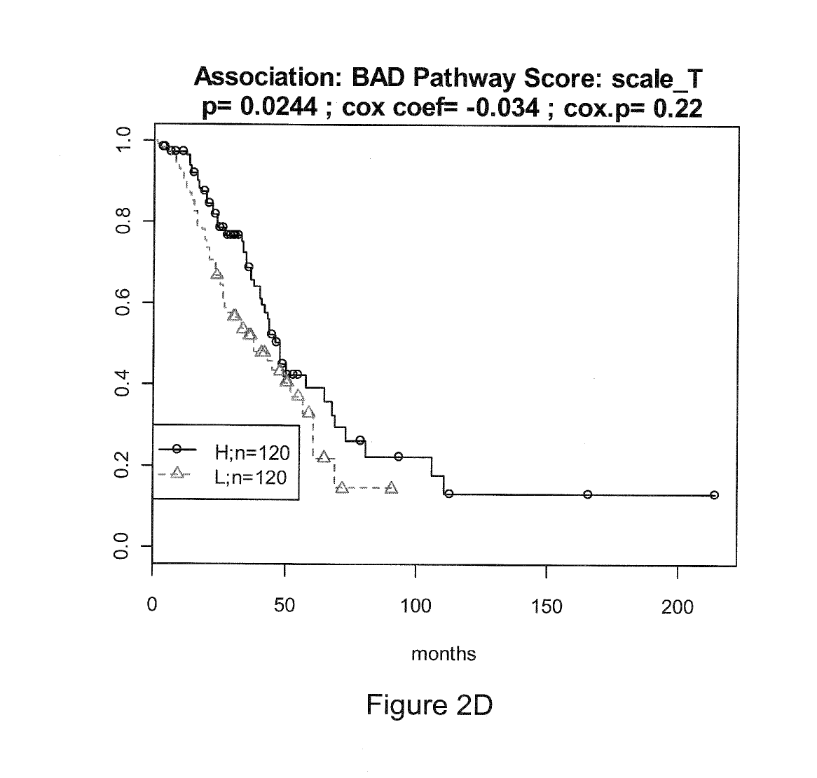

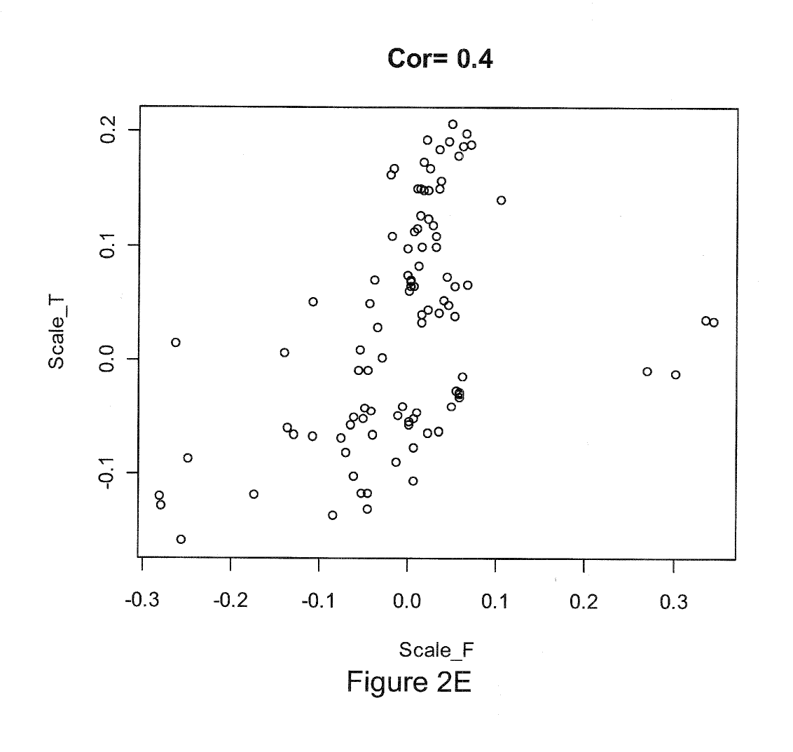

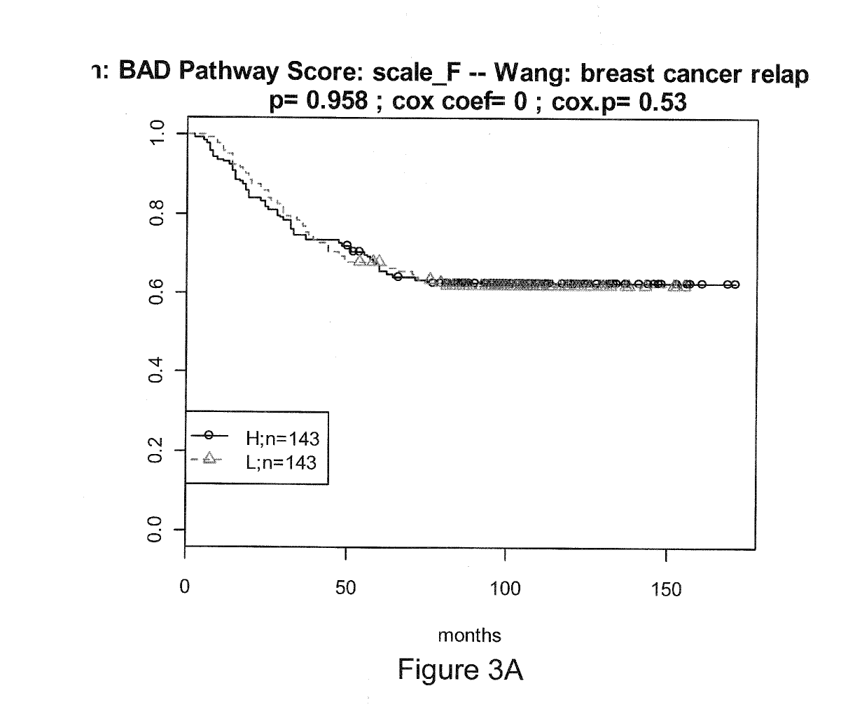

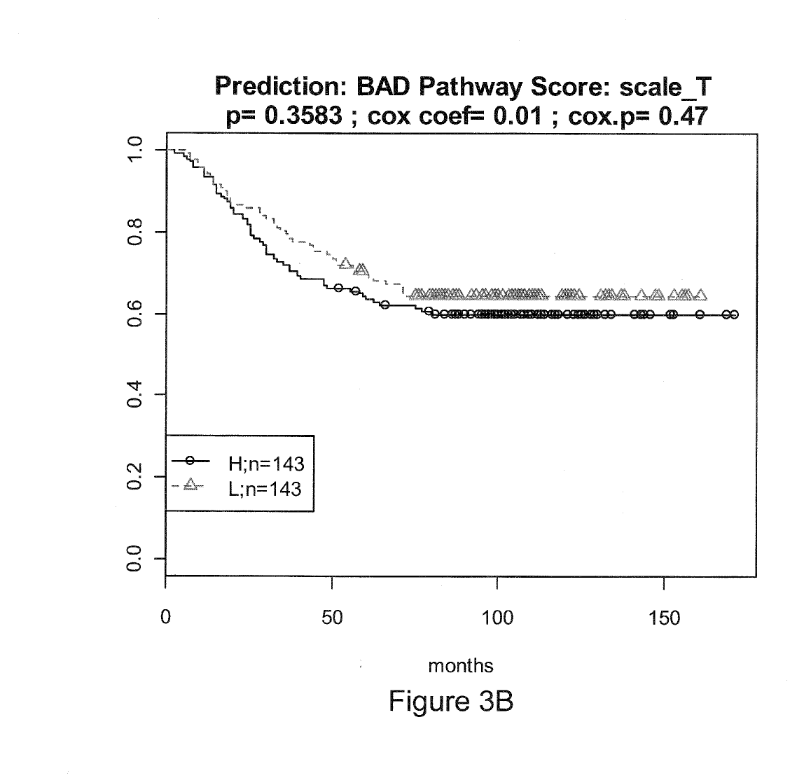

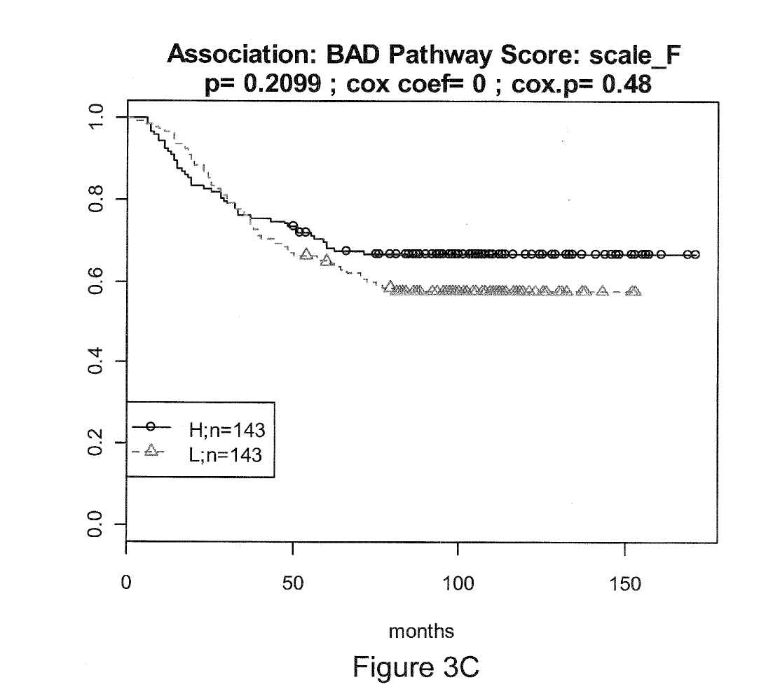

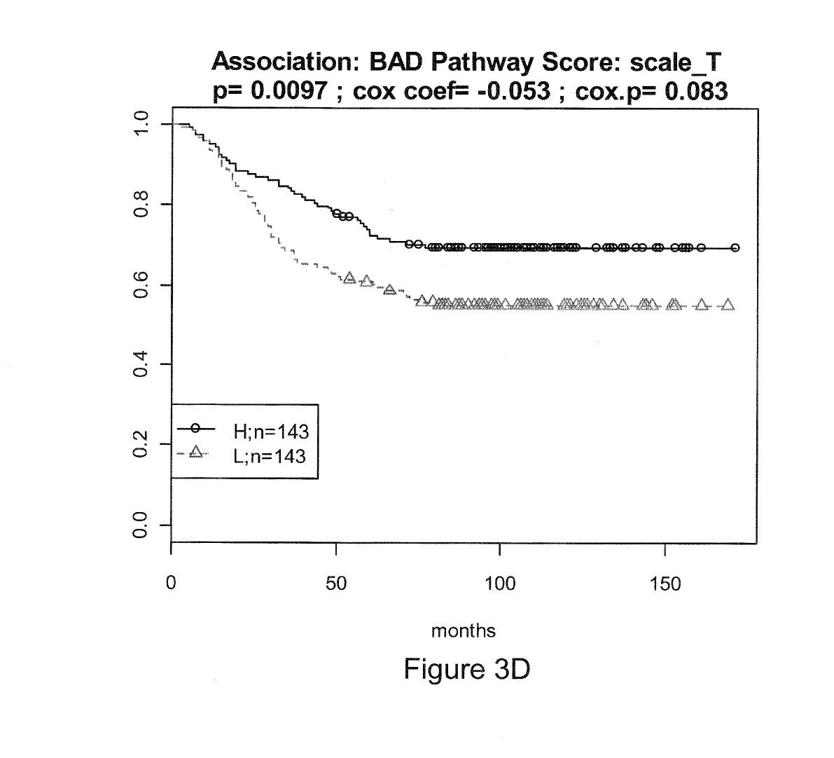

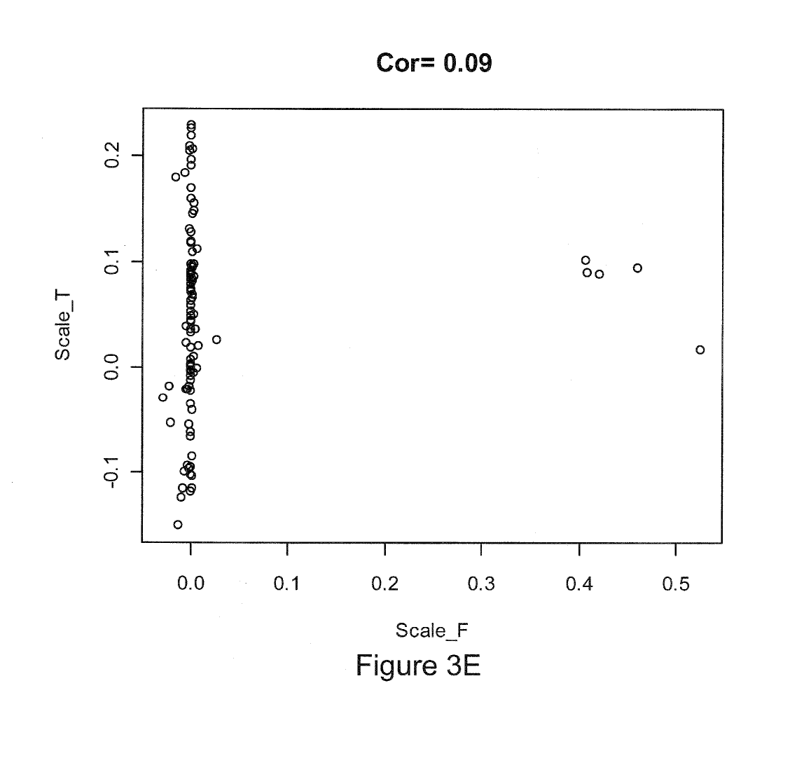

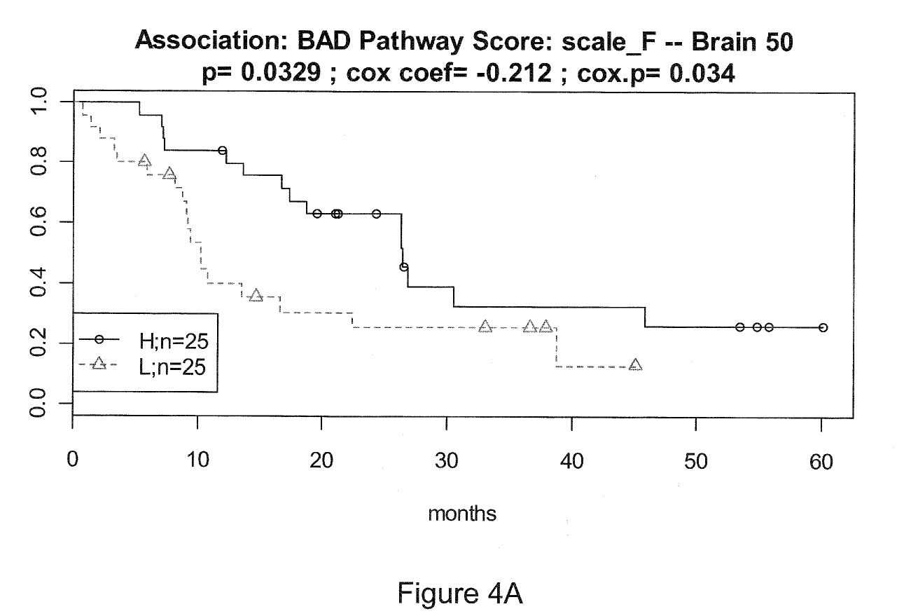

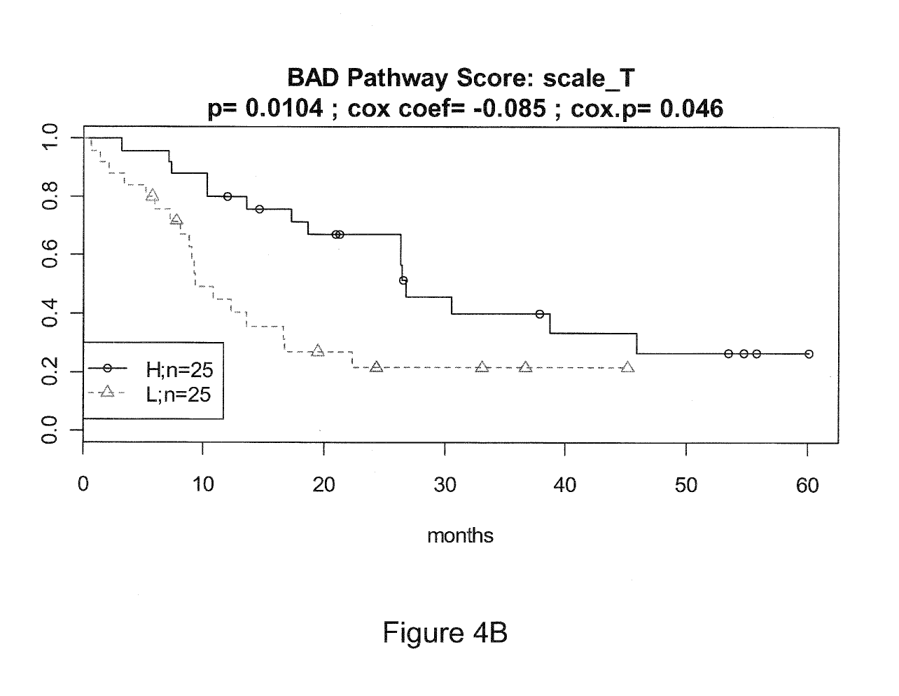

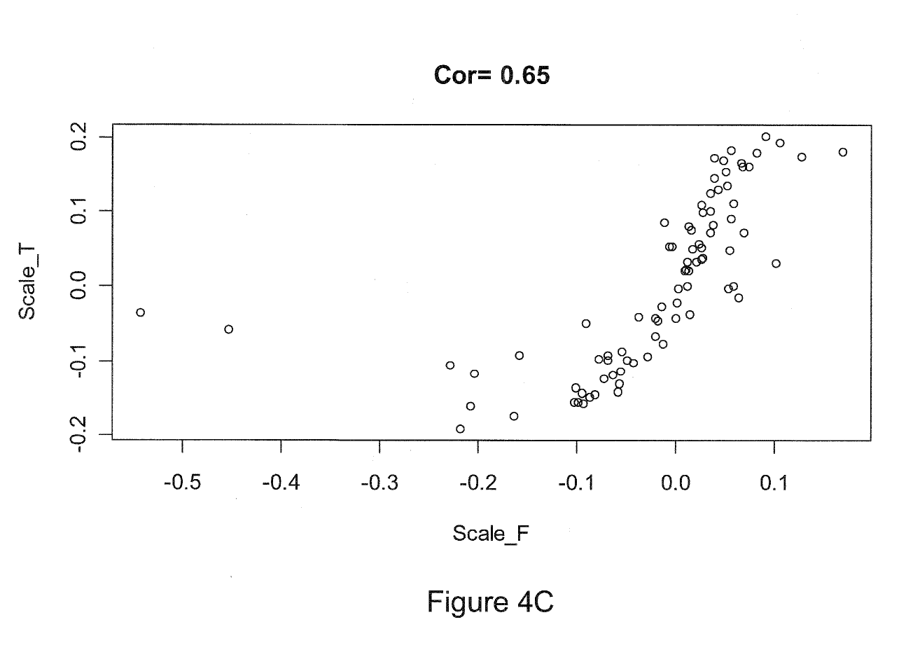

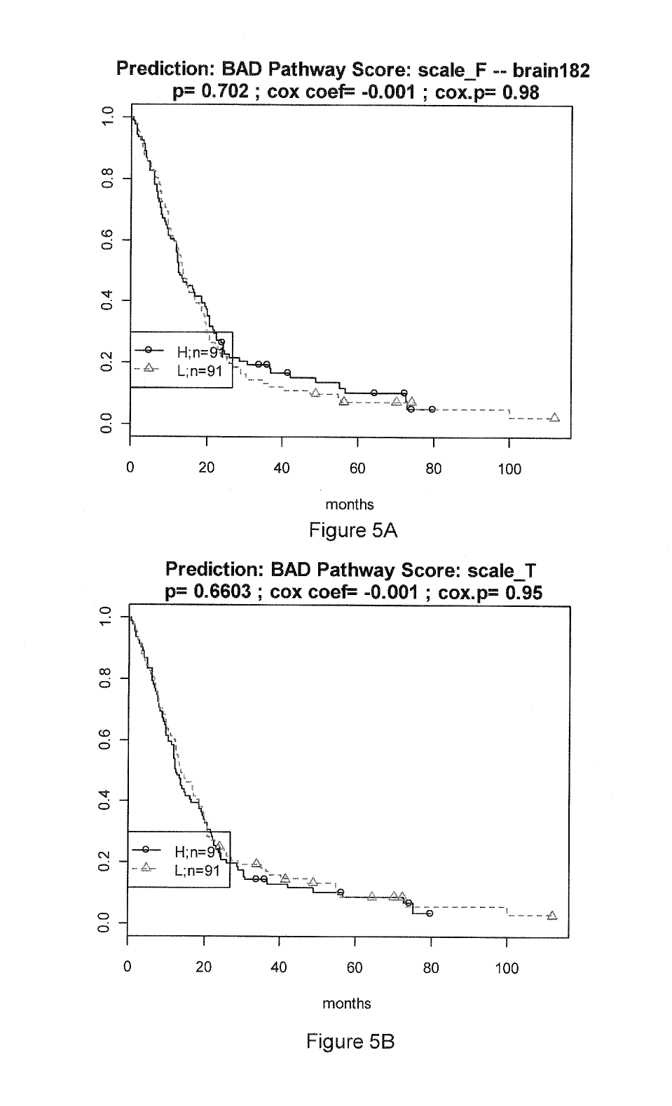

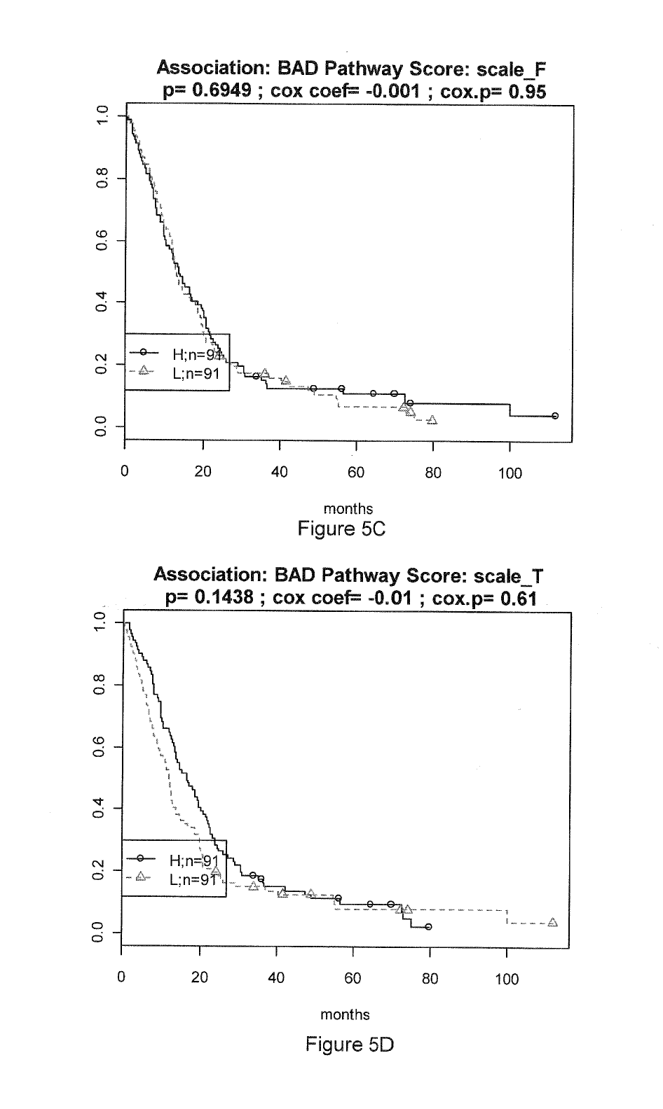

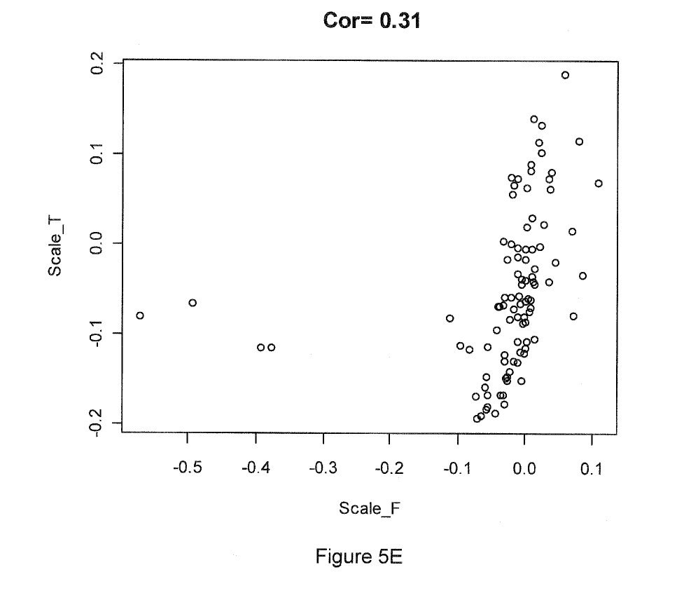

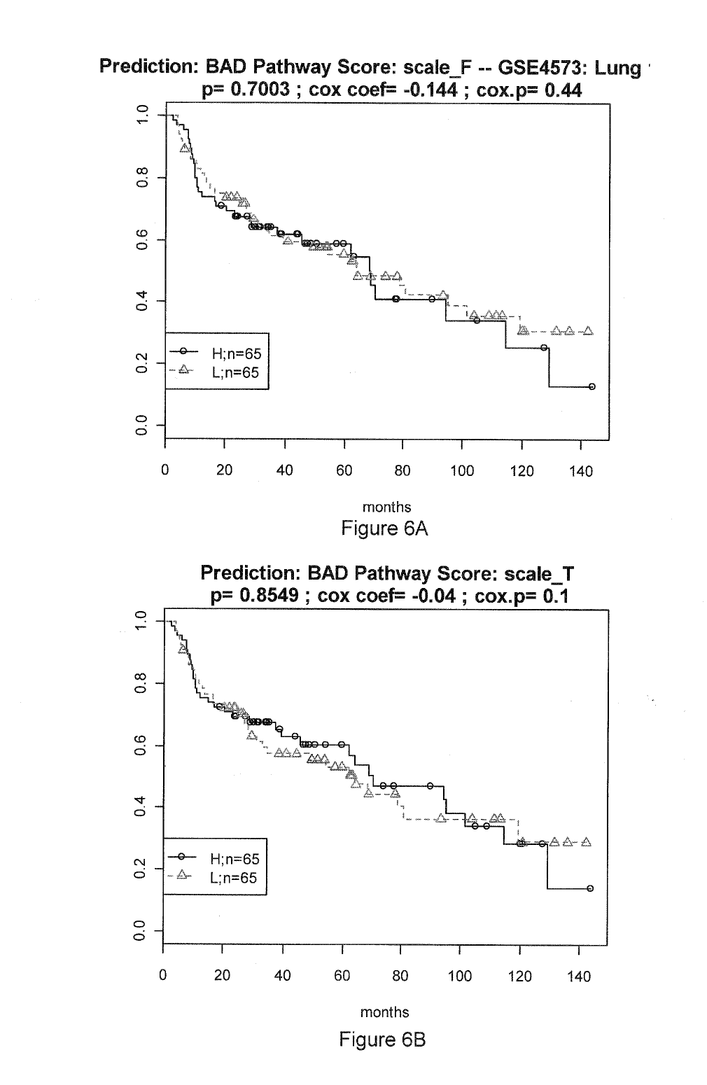

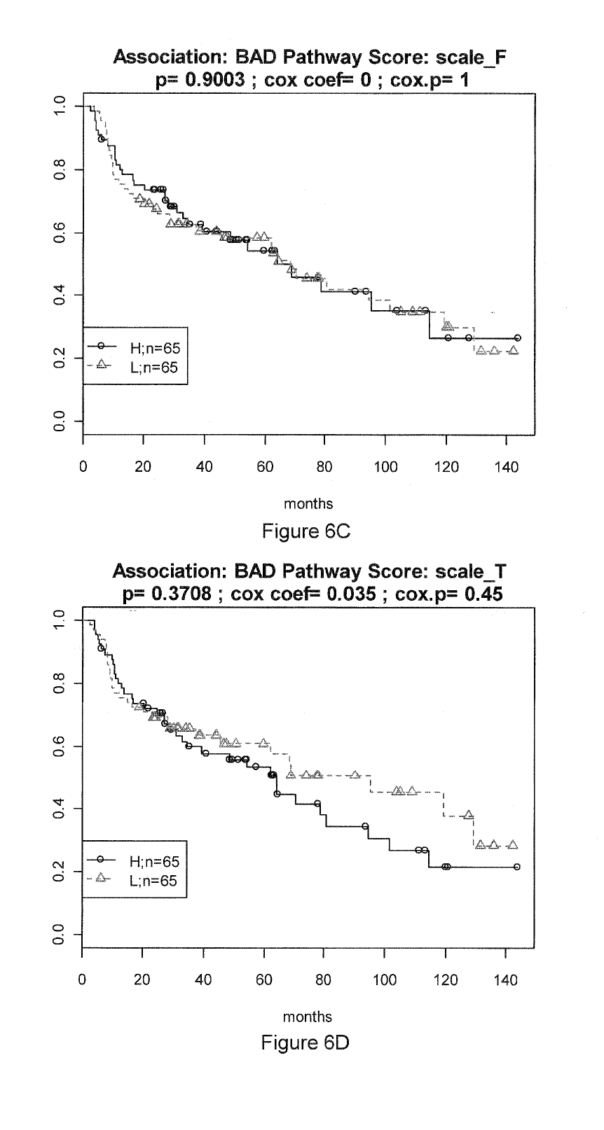

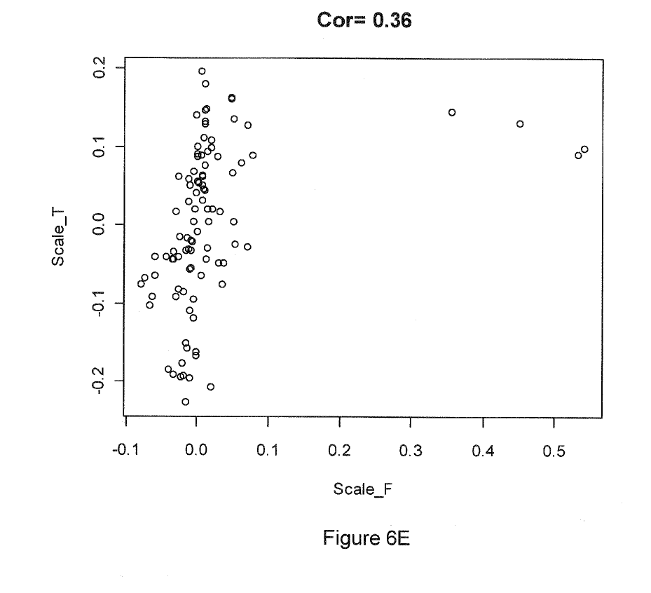

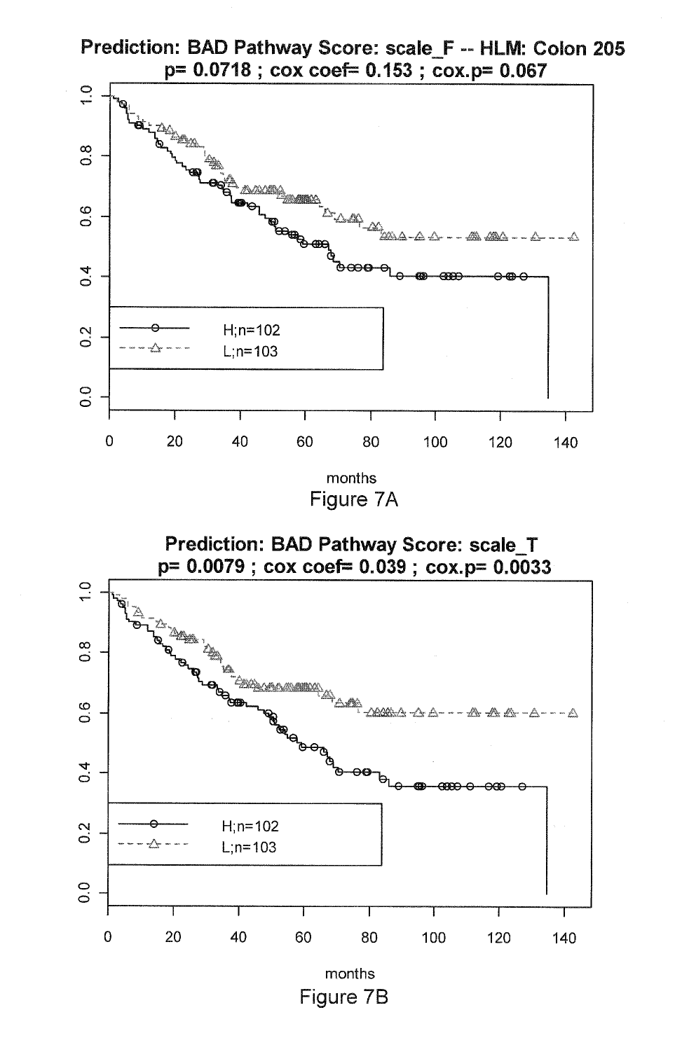

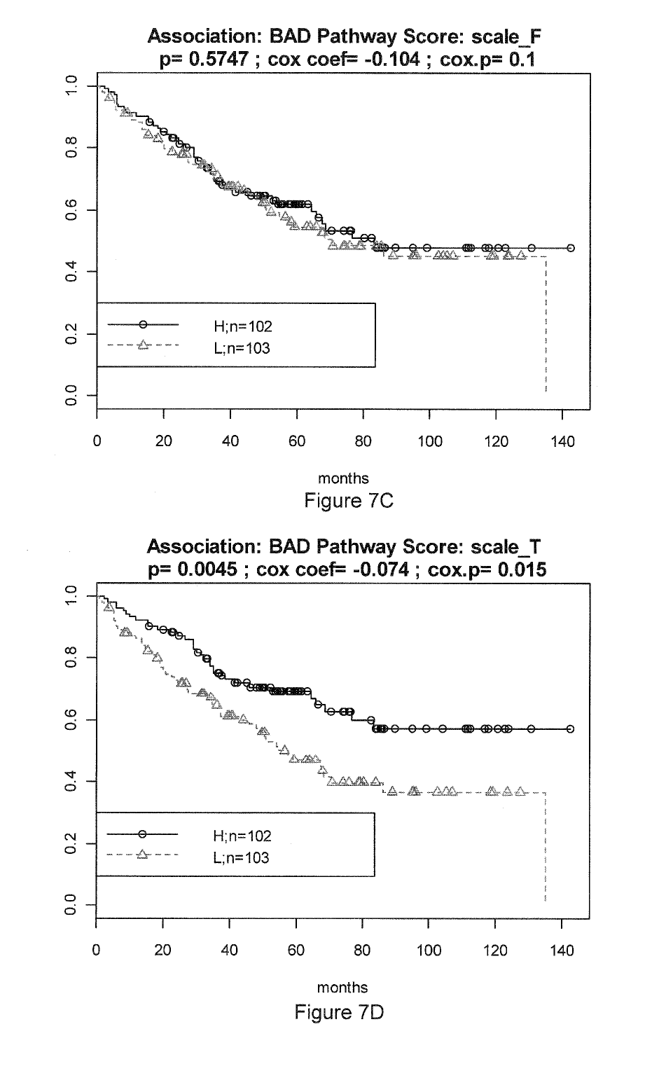

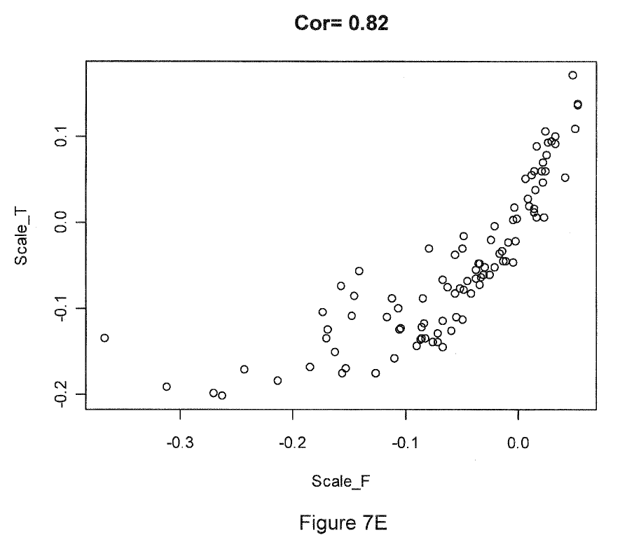

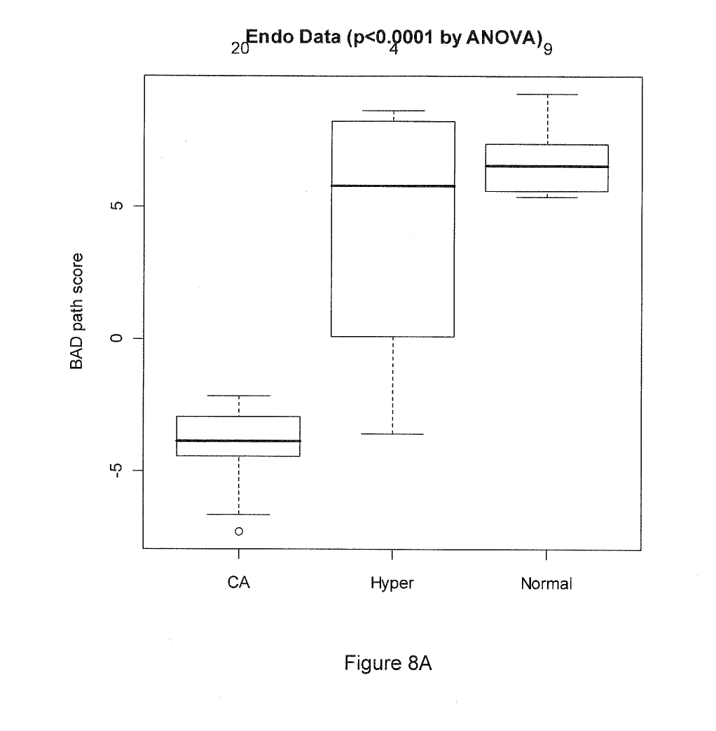

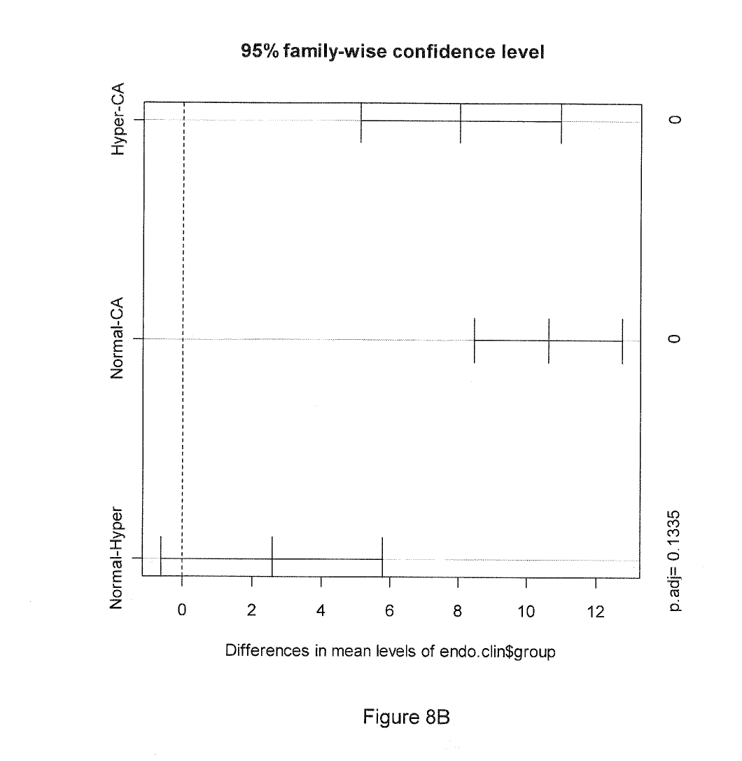

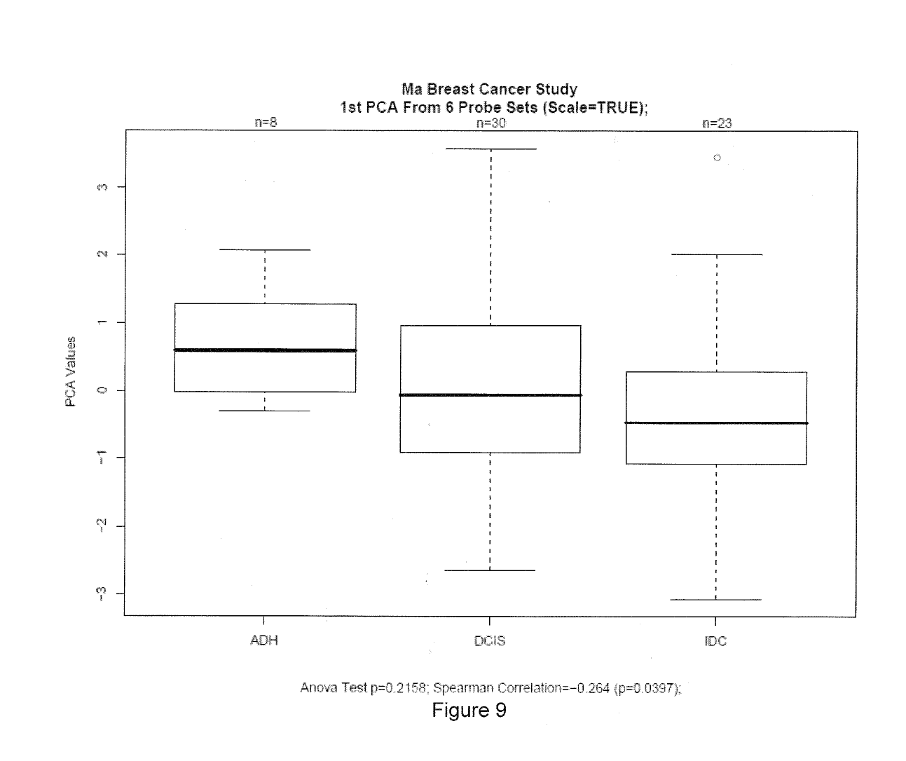

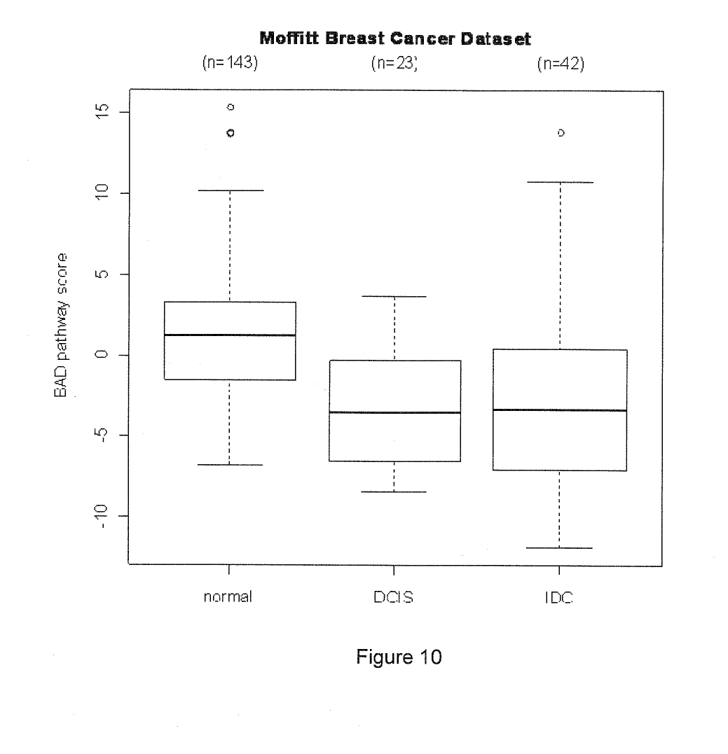

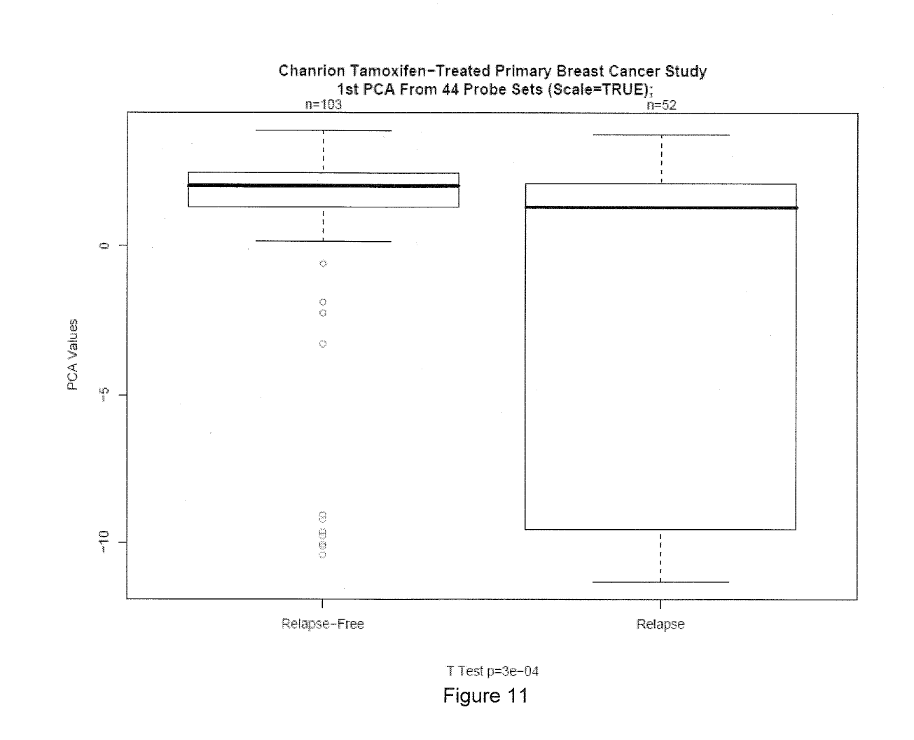

The BAD pathway score showed a statistically significant association with overall survival, using the Log-rank (Mantel Cox) Test (p<0.0001; FIG. 1A). As might be expected, patients who experienced a complete response (CR) to primary platinum-based therapy following primary surgery experienced superior survival (p<0.0001; Log-rank, Mantel-Cox test) compared to those patients who experienced an in-complete response (IR) to primary surgery and platinum-based therapy (FIG. 1B). Evaluating genomic data from those patients who experienced a CR (n=104), revealed that CR patients with a BAD pathway score (PC1) higher than the median score (defined as “High BAD pathway score, PC1”), had a statistically significant survival advantage (p=<0.0001; Log-rank, Mantel-Cox test) compared to those CR patients with a BAD pathway score (PC1) lower than the median score (defined as “Low BAD pathway score, PC1”; FIG. 1C). Similarly, evaluating genomic data from those patients who experienced an IR (n=39), revealed that 1R patients with a high BAD pathway score (PC1), had a survival advantage compared to those IR patients with a low BAD pathway score (FIG. 1D), though this survival difference did not reach statistical significance (p=0.6072; Log-rank, Mantel-Cox test). These differences are also illustrated in FIG. 1E.

Patients who underwent primary surgical cytoreduction (debulking) and were left with residual disease less than 1 cm in greatest diameter (optimal debulking), experienced mean survival of 62 months compared to 50 months (p=0.1942; FIG. 1F) for those patients who underwent primary surgical debulking and were left with residual disease greater than 1 cm in greatest diameter (sub-optimal debulking) For those 74 patients who underwent optimal debulking, evaluation of BAD pathway scores revealed a superior survival for patients with high versus low BAD pathway scores (p=0.0007, FIG. 1G). This pattern was also observed for the 68 patients who experienced sub-optimal debulking; in this group, patients with high BAD pathway scores experienced a superior survival compared to those patients with a low BAD pathway score (p=0.0288, FIG. 1H). When debulking status and BAD pathway score data was integrated, and evaluated with overall survival it became clear that patients who underwent optimal debulking with high BAD pathway scores had the best survival. Consistently, worst survival was observed in patients who underwent sub-optimal debulking with low BAD pathway scores (FIG. 1I).

B. GSE9891: 240 Patients with Ovarian Cancer Treated in Australia (the Australian OVCA Dataset):

The pathway score showed a statistically significant association with overall survival, using the median cutoff BAD pathway score (p=0.024; log-rank test).

The 1^{st} column is the results for prediction (derived from Moffitt dataset) using un-standardized expression data (scale=F) and standardized expression data (scale=T).

The 2^{nd} column is the results for association using un-standardized expression data (scale=F) and standardized expression data (scale=T).

The 3^{rd} column is the correlation of weight (loading coefficient) between un-standardized versus standardized expression data.

Correlation matrix of predicted weights (derived from Moffitt dataset) and the weights using the own dataset:

$$\begin{matrix} & scale\_F & scale\_T & scale\_F & scale\_T \\ scale\_F & 1.00 & 0.40 & - 0.08 & - 0.02 \\ scale\_T & 0.40 & 1.00 & - 0.36 & - 0.53 \\ scale\_F & - 0.08 & - 0.36 & 1.00 & 0.84 \\ scale\_T & - 0.02 & - 0.53 & 0.84 & 1.00\end{matrix}$$

C. GSE2034: 286 Patients with Breast Cancer, and Followed for Both Relapse Free Survival and Also Distant Metastasis Free Survival (the Breast Cancer 286 Dataset).

The inventors analyzed 286 cell files, all processed on Affymetrix U133A chips for relapse free survival (RFS). Kaplan Meier survival analysis (survival time represented as “time to relapse or last follow up”) demonstrated a statistically significant association with relapse free survival, using the median cutoff BAD pathway score (p=0.024; log-rank test).

The pathway score showed a statistically significant association with overall survival, using the median cutoff BAD pathway score (p=0.00097; log-rank test).

The 1^{st} column is the results for prediction (derived from Moffitt dataset) using un-standardized expression data (scale=F) and standardized expression data (scale=T).

The 2^{nd} column is the results for association using un-standardized expression data (scale=f) and standardized expression data (scale=T).

The 3^{rd} column is the correlation of weight (loading coefficient) between un-standardized versus standardized expression data.

Correlation matrix of predicted weights (derived from Moffitt dataset) and the weights using the own dataset:

$$\begin{matrix} & scale\_F & scale\_T & scale\_F & scale\_T \\ scale\_F & 1.00 & 0.09 & 0.00 & 0.15 \\ scale\_T & 0.09 & 1.00 & 0.00 & - 0.09 \\ scale\_F & 0.00 & 0.00 & 1.00 & 0.84 \\ scale\_T & 0.15 & - 0.09 & 0.84 & 1.00\end{matrix}$$

D. The 50 Brain Cancers:

The pathway score showed a statistically significant association with overall survival, using the median cutoff BAD pathway score (p=0.01; log-rank test).

The 1^{st} row is the results for association using un-standardized expression data (scale=F) and standardized expression data (scale=T).

The 2^{nd} row is the correlation of weight (loading coefficient) between un-standardized versus standardized expression data.

E. The 182 Brain Cancers:

The pathway score did not show a statistically significant association or prediction with overall survival, using the median cutoff BAD pathway score.

The 1^{st} column is the results for prediction (derived from Moffitt dataset) using un-standardized expression data (scale=F) and standardized expression data (scale=T).

The 2^{nd} column is the results for association using un-standardized expression data (scale=F) and standardized expression data (scale=T).

The 3^{rd} column is the correlation of weight (loading coefficient) between un-standardized versus standardized expression data.

Correlation matrix of predicted weights (derived from Moffitt dataset) and the weights using the own dataset:

$$\begin{matrix} & scale\_F & scale\_T & scale\_F & scale\_T \\ scale\_F & 1.00 & 0.31 & 0.11 & 0.10 \\ scale\_T & 0.31 & 1.00 & 0.31 & 0.32 \\ scale\_F & 0.11 & 0.31 & 1.00 & 0.84 \\ scale\_T & 0.10 & 0.32 & 0.84 & 1.00\end{matrix}$$

F. GSE4573 (Lung 130):

The pathway score did not show a statistically significant association or prediction with overall survival, using the median cutoff BAD pathway score.

The 1^{st} column is the results for prediction (derived from Moffitt dataset) using un-standardized expression data (scale=F) and standardized expression data (scale=T).

The 2^{nd} column is the results for association using un-standardized expression data (scale=F) and standardized expression data (scale=T).

The 3^{rd} column is the correlation of weight (loading coefficient) between un-standardized versus standardized expression data.

Correlation matrix of predicted weights (derived from Moffitt dataset) and the weights using the own dataset:

$$\begin{matrix} & scale\_F & scale\_T & scale\_F & scale\_T \\ scale\_F & 1.00 & 0.36 & - 0.17 & - 0.20 \\ scale\_T & 0.36 & 1.00 & - 0.45 & - 0.39 \\ scale\_F & - 0.17 & - 0.45 & 1.00 & 0.84 \\ scale\_T & - 0.20 & - 0.39 & 0.84 & 1.00\end{matrix}$$

G. 205 Patients with Colon Cancer Treated at Moffitt Cancer Center (The MCC Colon Dataset):

High BAD pathway score was associated with favorable survival.

H. 33 Endometrial Samples.

Boren et al. August; 110(2):206-15. Gynecol Oncol. 2008. (The MCC endometrial dataset): BAD pathway score was associated with the transition from normal to atypical hyperplasia to invasive cancer. The BAD pathway score was highest in normal endometrial tissue samples and lowest in invasive endometrial cancer samples. Endometrial atypical hyperplasia samples had a score intermediate between normal and invasive cancer samples.

I. 61 Breast Samples (Atypical Ductal Hyperplasia, Ductal Carcinoma In Situ, and Invasive Ductal Carcinoma) (The Ma et al. Dataset):

BAD pathway score was associated with the transition from atypical ductal hyperplasia (ADH) to ductal carcinoma in situ (DCIS) to invasive ductal carcinoma (IDC). The BAD pathway score was highest in atypical ductal hyperplasia tissue samples and lowest in invasive ductal carcinoma samples. Ductal carcinoma in situ, samples had a score intermediate between atypical ductal hyperplasia and invasive cancer samples.

J. 197 Breast Samples (Normal, Ductal Carcinoma In Situ, and Invasive Ductal Carcinoma) (The MCC 197 Breast Dataset):

BAD pathway score was associated with the transition from normal breast tissue to ductal carcinoma in situ (DCIS) to invasive ductal carcinoma (IDC). The BAD pathway score was highest in normal breast tissue samples and lowest in invasive ductal carcinoma samples. Ductal carcinoma in situ, samples had a score intermediate between normal and invasive cancer samples.

K. Chanrion Tamoxifen-Treated Primary Breast Cancer Study (Relapse-Free vs. Relapse) (Charion dataset):

BAD pathway score was associated with relapse-free survival. The BAD pathway score was highest in relapse-free breast cancer samples and lowest in breast cancer samples that went on to relapse. Relapse-Free vs. Relapse: p=0.0003.

The inventors have identified a genomic signature, based on gene expression of members of the BAD pathway that can discriminate between patients with longer versus shorter survival from many human cancers. This signature, developed using in vitro analysis of ovarian cancer cells, has shown statistical association with overall survival or relapse-free survival in two large ovarian cancer patient datasets, one brain cancer datasets, and a large breast cancer dataset. This signature has clinical utility as a prognostic biomarker for patients with many types of cancer. Moreover, it can enable physicians to identify patients with poor prognosis, who may benefit from more aggressive therapy. In this regard, the BAD pathway is a viable therapeutic target.

The inventors have demonstrated previously that manipulation of the phosphorylation status of BAD, either directly or indirectly, influences sensitivity to cytotoxic therapy. Thus, for patients that have evidence of poor prognosis disease on the basis of their BAD pathway score, it may be beneficial to add additional agents that target the BAD pathway including those listed in Table 2.

TABLE 2

Drugs that target the BAD pathway
Retinoic Acid
Panitumumab
BMS690514
IMC11F8
ICR62
Quercetin
SU6668
Erlotinib
Vatalanib
Vandetanib
Cetuximab
Pelitinib
BIBX1382
ARRY334543
Lapatinib
EMD55900
Suramin
Dovitinib
D69491
Semaxanib
Levodopa
RG83852
Canertinib
BMS599626
TGF Alpha
PE40
MDX214
AV412
XL647
Nimotuzumab
Midostaurin
DAB389EGF
Genistein
Gefitinib
Metformin
Zalutumumab
AEE788
BIBW2992
HKI272
PKI166
Matuzumab
PD153035
DPCPX
Lypressin
Theophylline
Flavoxate
Midostaurin
Docosahexaendic Acid
(all-Z) Eicosapentaenoic Acid
Colchicine
Vatalanib
Roscovitine
Cep5214
Mictefosine
P276-00
Phorbol 12-Myristate 13- Alestate
Suramin
Estradiol
(3R, 4S) Flavopiridol
Terameprolol
PD153035
(R)-Rescovitine
Genistein
Quercetin
R547
Etodozal Ralemic
Nitris Oxide (NO)
Imatinib
Troglitazone
Pallitaxel
Vatalinib
Celecoxib
Ruboxistaurin
XL418
Quercetin
Ave1642
Masoprolol
Pudophyllotoxin
SU 6668
Semaxanib
Mecasermin
Onapristone
AM111
Adenine
Vatalanib
Idarubicin
Docetaxel
Estradiol
Fluoxetine
Curcumin
Ethinyz Estradiol
Obatoclax
Glutamione
Metoxantrone
Chlorambucil
Mafosfamide
Calcitriol
Fluoxetine
Cisplatin
Pirfenidone
Cyclosporina
Rituximab
PD0325901
Arry438162
CI1040
Arry142886
Chloroquine
Sorafenib

TABLE 3
NCI60 compounds with correlation to
BAD PC1 score (adjusted p<0.01)

| NSC.Code | pearson.score | p.value | fdr.adjust |
|---|---|---|---|
| 150412 | 0.679094064 | 4.64E−09 | 0.000189405 |
| 716015 | 0.631125871 | 1.09E−07 | 0.00222469 |
| 623794 | 0.663630802 | 2.02E−07 | 0.002520635 |
| 673835 | −0.63062654 | 2.47E−07 | 0.002520635 |
| 726086 | 0.623057396 | 3.77E−07 | 0.002687317 |
| 706009 | 0.76245378 | 3.95E−07 | 0.002687317 |
| 684389 | 0.628549488 | 4.67E−07 | 0.002723277 |
| 76751 | 0.598458271 | 8.80E−07 | 0.00427668 |
| 617277 | 0.586793469 | 1.04E−06 | 0.00427668 |
| 651010 | 0.629418098 | 1.27E−06 | 0.00427668 |
| 325661 | 0.600216582 | 1.27E−06 | 0.00427668 |
| 613680 | 0.604271973 | 1.31E−06 | 0.00427668 |
| 663593 | 0.58762312 | 1.54E−06 | 0.00427668 |
| 690147 | 0.63384605 | 1.73E−06 | 0.00427668 |
| 683901 | 0.590742553 | 2.06E−06 | 0.00427668 |
| 665712 | 0.581196629 | 2.13E−06 | 0.00427668 |
| 686974 | 0.588848812 | 2.26E−06 | 0.00427668 |
| 669637 | 0.64433033 | 2.36E−06 | 0.00427668 |
| 666700 | 0.595378696 | 2.57E−06 | 0.00427668 |
| 666991 | 0.575039479 | 2.89E−06 | 0.00427668 |
| 666463 | 0.586794538 | 3.11E−06 | 0.00427668 |
| 115493 | 0.57306156 | 3.18E−06 | 0.00427668 |
| 366369 | 0.594692189 | 3.34E−06 | 0.00427668 |
| 669630 | 0.614453452 | 3.39E−06 | 0.00427668 |
| 721859 | 0.571570849 | 3.42E−06 | 0.00427668 |
| 681484 | 0.579470907 | 3.56E−06 | 0.00427668 |
| 112929 | 0.566249873 | 3.61E−06 | 0.00427668 |
| 655504 | 0.623835 | 3.62E−06 | 0.00427668 |
| 647579 | 0.564472821 | 3.93E−06 | 0.00427668 |
| 57850 | 0.577300551 | 3.95E−06 | 0.00427668 |
| 678357 | 0.567532277 | 4.15E−06 | 0.00427668 |
| 698052 | 0.579409066 | 4.42E−06 | 0.00427668 |
| 677746 | 0.574765102 | 4.45E−06 | 0.00427668 |
| 664228 | 0.570238772 | 4.48E−06 | 0.00427668 |
| 668434 | 0.619113652 | 4.50E−06 | 0.00427668 |
| 719276 | 0.565747552 | 4.52E−06 | 0.00427668 |
| 702156 | 0.578589008 | 4.59E−06 | 0.00427668 |
| 664016 | 0.557065959 | 4.60E−06 | 0.00427668 |
| 683654 | 0.564108575 | 4.88E−06 | 0.00427668 |
| 134054 | 0.591347176 | 4.89E−06 | 0.00427668 |
| 678359 | 0.563950491 | 4.92E−06 | 0.00427668 |
| 122896 | 0.572441552 | 4.96E−06 | 0.00427668 |
| 45541 | 0.567872223 | 5.00E−06 | 0.00427668 |
| 676998 | 0.559228748 | 5.04E−06 | 0.00427668 |
| 697702 | 0.571502132 | 5.18E−06 | 0.00427668 |
| 669629 | 0.599404695 | 5.34E−06 | 0.00427668 |
| 675213 | 0.566347617 | 5.37E−06 | 0.00427668 |
| 683900 | 0.574866924 | 5.46E−06 | 0.00427668 |
| 665670 | 0.574674155 | 5.51E−06 | 0.00427668 |
| 682742 | 0.570135001 | 5.52E−06 | 0.00427668 |
| 658199 | 0.565675357 | 5.55E−06 | 0.00427668 |
| 663998 | 0.561111887 | 5.62E−06 | 0.00427668 |
| 661098 | 0.560676259 | 5.73E−06 | 0.00427668 |
| 674192 | 0.551375546 | 6.02E−06 | 0.00427668 |
| 84922 | 0.563443059 | 6.15E−06 | 0.00427668 |
| 58571 | 0.596232037 | 6.17E−06 | 0.00427668 |
| 743379 | 0.55844017 | 6.36E−06 | 0.00427668 |
| 693426 | 0.571446878 | 6.39E−06 | 0.00427668 |
| 683283 | 0.562317938 | 6.48E−06 | 0.00427668 |
| 679196 | 0.553254784 | 6.67E−06 | 0.00427668 |
| 682071 | 0.565922854 | 6.71E−06 | 0.00427668 |
| 661813 | 0.552946973 | 6.76E−06 | 0.00427668 |
| 661906 | 0.552946973 | 6.76E−06 | 0.00427668 |
| 661914 | 0.552946973 | 6.76E−06 | 0.00427668 |
| 661101 | 0.561223355 | 6.81E−06 | 0.00427668 |
| 177407 | 0.546581586 | 7.51E−06 | 0.004644821 |
| 661096 | 0.553875176 | 7.84E−06 | 0.004730318 |
| 718565 | 0.549614269 | 7.88E−06 | 0.004730318 |
| 694277 | 0.575031223 | 8.24E−06 | 0.00479635 |
| 674611 | 0.544380818 | 8.31E−06 | 0.00479635 |
| 655460 | 0.561036386 | 8.37E−06 | 0.00479635 |
| 669580 | 0.569787105 | 8.46E−06 | 0.00479635 |
| 705894 | 0.569325601 | 8.64E−06 | 0.004831299 |
| 661102 | 0.555235708 | 8.94E−06 | 0.004839318 |
| 661103 | 0.555235708 | 8.94E−06 | 0.004839318 |
| 663313 | 0.555068653 | 9.01E−06 | 0.004839318 |
| 113943 | 0.544936704 | 9.75E−06 | 0.005153525 |
| 734976 | 0.557096033 | 9.98E−06 | 0.005153525 |
| 666981 | 0.570437585 | 1.01E−05 | 0.005153525 |
| 688931 | 0.580033753 | 1.01E−05 | 0.005153525 |
| 698965 | 0.560871642 | 1.03E−05 | 0.005190691 |
| 154020 | 0.539334358 | 1.05E−05 | 0.005226951 |
| 722509 | 0.555587479 | 1.07E−05 | 0.005248286 |
| 602881 | 0.542752375 | 1.08E−05 | 0.005248286 |
| 697567 | 0.614847914 | 1.15E−05 | 0.005442667 |
| 629108 | 0.567349704 | 1.16E−05 | 0.005442667 |
| 716421 | 0.549372677 | 1.16E−05 | 0.005442667 |
| 118767 | 0.562032596 | 1.19E−05 | 0.005519977 |
| 38297 | 0.535838655 | 1.22E−05 | 0.005578733 |
| 108944 | 0.548030793 | 1.23E−05 | 0.005578733 |
| 658775 | 0.589632204 | 1.29E−05 | 0.005639052 |
| 31678 | 0.546663177 | 1.31E−05 | 0.005639052 |
| 21970 | 0.55077833 | 1.32E−05 | 0.005639052 |
| 715754 | 0.542149647 | 1.33E−05 | 0.005639052 |
| 668422 | 0.599905947 | 1.33E−05 | 0.005639052 |
| 677206 | 0.533930817 | 1.33E−05 | 0.005639052 |
| 691982 | 0.578550625 | 1.34E−05 | 0.005639052 |
| 668448 | 0.593438899 | 1.38E−05 | 0.005667243 |
| 649240 | 0.53301518 | 1.39E−05 | 0.005667243 |
| 671902 | 0.532956664 | 1.39E−05 | 0.005667243 |
| 667746 | 0.553459735 | 1.42E−05 | 0.005667243 |
| 721662 | 0.54886341 | 1.43E−05 | 0.005667243 |
| 711669 | 0.532245326 | 1.43E−05 | 0.005667243 |
| 717821 | 0.535136051 | 1.51E−05 | 0.005853434 |
| 683604 | 0.551928679 | 1.52E−05 | 0.005853434 |
| 710352 | 0.565619525 | 1.52E−05 | 0.005853434 |
| 677028 | 0.532395888 | 1.70E−05 | 0.006460209 |
| 663781 | 0.536107501 | 1.72E−05 | 0.006460209 |
| 674495 | 0.527476698 | 1.77E−05 | 0.006460209 |
| 665007 | 0.543934743 | 1.77E−05 | 0.006460209 |
| 700202 | 0.531243357 | 1.78E−05 | 0.006460209 |
| 618495 | 0.58727392 | 1.78E−05 | 0.006460209 |
| 716186 | 0.55702919 | 1.80E−05 | 0.006460209 |
| 700132 | 0.539162331 | 1.81E−05 | 0.006460209 |
| 701994 | 0.526774428 | 1.82E−05 | 0.006460209 |
| 694859 | 0.534206264 | 1.87E−05 | 0.006580466 |
| 704548 | 0.540691947 | 2.03E−05 | 0.007082444 |
| 704225 | 0.531774888 | 2.07E−05 | 0.007160797 |
| 665962 | 0.535376032 | 2.12E−05 | 0.007272134 |
| 707071 | 0.530139175 | 2.22E−05 | 0.0075517 |
| 674068 | 0.538151709 | 2.26E−05 | 0.007624231 |
| 684350 | 0.533474201 | 2.30E−05 | 0.007695574 |
| 658004 | 0.533313456 | 2.32E−05 | 0.007699382 |
| 691422 | 0.524106713 | 2.42E−05 | 0.007902752 |
| 683610 | 0.540803917 | 2.42E−05 | 0.007902752 |
| 670117 | 0.569053248 | 2.44E−05 | 0.007904825 |
| 711670 | 0.517803473 | 2.67E−05 | 0.008581843 |
| 625587 | 0.517397776 | 2.71E−05 | 0.008642359 |
| 11243 | 0.581960375 | 2.75E−05 | 0.008701938 |
| 308819 | 0.519633254 | 2.91E−05 | 0.0091374 |
| 691538 | 0.519263012 | 2.96E−05 | 0.00922345 |
| 676808 | 0.522898382 | 3.01E−05 | 0.009308197 |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Chen D T, Nasir A, Culhane A, Venkataramu C, Fulp W, Rubio R, Wang T, Agrawal D, McCarthy S M, Gruidl M, Bloom G, Anderson T, White J, Quackenbush J, and Yeatman T, Proliferative genes dominate malignancy-pathway gene signature in histologically-normal breast tissue. Breast cancer research and treatment, 2009. PMID:19266279.
2. Chen D T, Nasir A, Venkataramu C, Fulp W, Gruidl M, and Yeatman T, Evaluation of malignancy-pathway gene signature in breast cancer patients. Breast cancer research and treatment, 2009. PMID:19306088.
3. Tothill R W, Tinker A V, George J, Brown R et al. Novel molecular subtypes of serous and endometrioid ovarian cancer linked to clinical outcome. Clin Cancer Res 2008 Aug. 15; 14(16):5198-208. PMID: 18698038 (Ovarian 286)
4. Carroll J S, Meyer C A, Song J, Li W et al. Genome-wide analysis of estrogen receptor binding sites. Nat Genet 2006 November; 38(11):1289-97. PMID: 17013392 (breast 286)
5. Nutt C L, Mani D R, Betensky R A, Tamayo P, Cairncross J G, Ladd C, Pohl U, Hartmann C, McLaughlin M E, Batchelor T T, Black P M, von Deimling A, Pomeroy S L, Golub T R, Louis D N. Gene expression-based classification of malignant gliomas correlates better with survival than histological classification. Cancer Res. 2003 Apr. 1; 63(7):1602-7.PMID: 12670911 (brain 50)
6. Lee Y, Scheck A C, Cloughesy T F, Lai A et al. Gene expression analysis of glioblastomas identifies the major molecular basis for the prognostic benefit of younger age. BMC Med Genomics 2008 Oct. 21; 1:52. PMID: 18940004 (brain 182)
7. Raponi M, Zhang Y, Yu J, Chen G et al. Gene expression signatures for predicting prognosis of squamous cell and adenocarcinomas of the lung. Cancer Res 2006 Aug. 1; 66(15):7466-72. PMID: 16885343 (lung 130)

### Example 2

### BAD Pathway Gene Signature as Determinant of Chemoresistance and Clinical Outcome

The development of chemoresistance dramatically affects clinical outcomes in cancer patients. As such, targeted therapies that enhance cancer cell sensitivity to cytotoxic agents can significantly improve these outcomes. The clinical consequences of acquired resistance to chemotherapy are exemplified in advanced-stage ovarian cancer (OVCA) patients, undoubtedly contributing to the high mortality associated with this disease. Although the majority of patients with OVCAs demonstrate remarkable sensitivity to platinum-based chemotherapy during primary therapy, most of these patients eventually develop platinum-resistant recurrent disease.^{1,2} Once platinum-resistance has developed, few active therapeutic options exist and patient survival is generally short-lived.³ In this context, platinum resistance is frequently viewed as a surrogate clinical marker for more generic chemoresistance, and it is likely that defining the molecular changes that drive the evolution of the platinum-resistant phenotype will contribute to a broader understanding of human cancer chemoresistance. Changes in cellular drug efflux, increased cellular glutathione levels, increased DNA repair, and drug tolerance have all been shown to contribute to platinum resistance.⁴⁻⁶ More recently, genomic studies have defined gene expression signatures that may discriminate between cancers that are innately chemosensitive versus chemoresistant.⁷⁻⁹ However, the genome-wide expression changes associated with the progression of a cancer cell from chemosensitive to chemoresistant are less clear, and the discrete biologic pathways that drive the process are unknown. Moreover, how these pathways influence clinical outcomes and their potential as therapeutic targets remain to be defined.

In this study, using OVCA as a model, the inventors measured the genomic changes associated with the development of chemoresistance and have evaluated the BCL2 antagonist of cell death (BAD) apoptotic pathway as an important determinant of human cancer response to therapy and clinical outcome and also as a potential therapeutic target. The inventors analyzed specimens and/or genomic data from 1,406 patients and 116 cancer cell lines. Genome-wide expression changes and cisplatin-resistance were evaluated in OVCA cell lines subjected to a total of 144 (cisplatin)-treatment/recovery cycles. Pathway analysis was performed on genes associated with increasing cisplatin-resistance. BAD protein phosphorylation was studied in patient samples and cell lines, and small interfering RNAs (siRNA) were used to explore the pathway as a therapeutic target. The inventors evaluated the influence of BAD-pathway expression on chemosensitivity and/or clinical outcome using genomic data from 60 human cancer cell lines and ovarian, breast, colon, and brain cancers from 1,258 patients.

The BAD pathway was associated with evolution of OVCA cell line cisplatin-resistance (P<0.001) and resistance of 7 human cancer cell types to 8 cytotoxic agents (P<0.05). OVCA chemoresistance was associated with BAD protein phosphorylation, and targeted siRNA modulation produced corresponding changes in chemosensitivity. Expression of a 47-gene BAD-pathway signature was associated with survival of 1,258 patients with ovarian, breast, colon, and brain cancer. The OVCA BAD-pathway signature survival advantage was independent of surgical cytoreductive status.

The BAD apoptosis pathway influences the sensitivity of human cancers to a variety of chemotherapies, likely via modulation of BAD-phosphorylation. The pathway has clinical relevance as a biomarker of therapeutic response, patient survival, and as a promising therapeutic target.

### Materials and Methods

Induction of In Vitro Platinum Resistance.

Acquisition and culture conditions for the expansion of OVCA cells lines (T8, OVCAR5, OV2008, IGROV1, C13, A2780S, A2780CP, and A2008) were reported previouslyl¹⁰. Cells were subjected to sequential treatment with increasing doses of cis-diammine-dichloroplatinum (cisplatin), using three dosing schedules resulting in 144 treatment/expansion cycles. Treatment schedules A, B, and C included three treatments with 1, 2, and 3 μg/mL cisplatin, respectively, followed by three treatments with 3, 4, and 5 μg/mL, respectively. Each cisplatin treatment was followed by a cell recovery/expansion phase. Both cisplatin-resistance and genome-wide expression changes were measured serially in each cell line at baseline and after 3 and 6 cisplatin-treatment/expansion cycles. Cisplatin-resistance was quantified using CellTiter-96 MTS proliferation assays (Fisher Scientific) and analyzed genome-wide expression using Affymetrix Human U133 Plus 2.0 GeneChips as previously described^{10,11} (Gene Expression Omnibus (GEO) accession number GSE23553).

Statistical Analysis of Cell Line Array Data.

Pearson correlation was used to identify genes associated with OVCA development of cisplatin-resistance (EC50). Expression was calculated using the robust multi-array average algorithm¹² implemented in Bioconductor (http://www.bioconductor.org) extensions to the R-statistical programming environment as described previously.¹³ Probe sets with expression ranges <2-fold (maximum/minimum) and control probes (i.e., AFFX_*probe sets) were excluded from the analysis. For each cell line, Pearson correlation coefficients were calculated for expression data and cisplatin EC50. Genes/probe sets demonstrating expression/EC50 correlations (IR>0.85) were subjected to biological pathway analysis using GeneGo/MetaCore™ software. Maps/pathways were identified using the GeneGo/MetaCore™ statistical test for significance (P<0.001).

Primary OVCA Patient Samples.

The inventors evaluated the role of BAD-pathway mRNA and protein levels in chemoresponse in 290 advanced-stage (III/IV) serous epithelial OVCAs, resected at the time of primary surgery from patients who would receive platinum-based therapy. All tissues, acquired with Institutional Review Board approval, were processed as previously reported.^{8,10} Microarray gene expression data (Affymetrix HG-U133A) were analyzed for 142 patients (114 samples previously reported⁸ and 28 Moffitt Cancer Center (MCC) samples; GEO accession number GSE23554). The inventors analyzed an additional 148 primary OVCAs (98 from MCC and 50 from University of Minnesota) for BAD-protein levels using immunofluorescence. Using medical record review, the inventors characterized all 290 OVCA samples as complete responders (CR) or incomplete responders (IR) to primary platinum-based therapy using criteria described previously⁸.

Statistical Analysis of Primary OVCA Genomic Data.

Probe sets differentially expressed between CR (n=101) and IR (n=41) samples were identified using Student's t-test (P<0.01) and subjected to GeneGo/MetaCore™ pathway analysis.

Characterization of the BAD-Apoptosis Pathway Proteins in Primary OVCAs and Cell Lines.

Total BAD, phosphorylated BAD (serine-112, -136, -155), non-phosphorylated BAD (Genscript), and BAD phosphatase PP2C/PPM1A (Santa Cruz Biotechnology) protein levels were evaluated in a subset of the cell line panel (8 cisplatin-treated OVCA cell lines) and in 148 primary OVCA samples (81 CR and 67 IR) by Western blot or by immunofluorescence as previously described^{14,15}.

siRNA Transfection.

RNA duplexes for PP2C/PPM1A (s10909 from ABI), cAMP-dependent protein kinase (PKA; 6406 from Cell Signaling), and vectors containing mutated BAD (pFlag-600, a kind gift from Dr. Hong Gang Wang, MCC) were transfected using the Nucleofector transfection kit, with non-targeting Silencer negative control #2 siRNA (ABI), according to manufacturer's protocols (Amaxa).

Analysis of Apoptotic Nuclei.

Morphologic assessment of condensed chromatin and fragmented DNA quantified percent apoptotic nuclei. Cells were fixed in 4% paraformaldehyde, and nuclei were stained with bis-benzimide trihydrochloride (0.5 μg/ml; Molecular Probes) and quantified using fluorescence microscopyl⁵.

Analysis of Data of 60 NCI Cell Lines.

Affymetrix HG-U133A expression and GI50 chemosensitivity data for the 60 NCI cancer cell lines (6 leukemia, 9 melanoma, 9 non-small cell lung, 7 colon, 6 central nervous system, 7 ovarian, 8 renal, 2 prostate, and 6 breast cancer cell lines) to cisplatin, carboplatin, cyclophosphamide, doxorubicin, gemcitabine, paclitaxel, docetaxel, and topotecan were obtained from NCI Web sites (http://discover.nci.nih.gov/cellminer/loadDownload.do and http://dtp.nci.nih.gov/dtpstandard/cancerscreeningdata/index.jsp). For each of the eight drugs, gene expression data from the most sensitive and resistant cell lines (cutoff=mean GI50+standard deviation) were compared using significance analysis of microarrays t-test (false discover rate of <20%)^{16,17} and subjected to GeneGo/MetaCore™ pathway analyses.

Development of a BAD-Pathway Gene Expression Signature.

Principal component analysis was used to derive a BAD-pathway gene expression signature with a corresponding “pathway score” that represents overall gene expression levels for BAD-pathway genes. The influence of the signature was evaluated in 7 external clinical-genomic expression datasets from 1,258 patients, including 1) 142 OVCA samples from MCC and Duke University Medical Center (North American OVCA dataset), 2) 238 OVCA samples from Melbourne, Australia¹⁸, 3) 205 colon cancers (MCC), 4) 50 malignant gliomas¹⁹, 5) 182 glioblastomas²⁰, 6) 286 breast cancers,^{21,22} and 7) 155 tamoxifen-treated breast cancers.²³ The association between BAD-pathway score (high versus low score based on median BAD-score cutoff) and clinical outcome was evaluated. Kaplan-Meier survival curves were generated, and high/low BAD-pathway score survival differences were evaluated using a log-rank test.

### Results

A. Expression of BAD-Pathway Genes Correlates with the Evolution of Platinum Resistance:

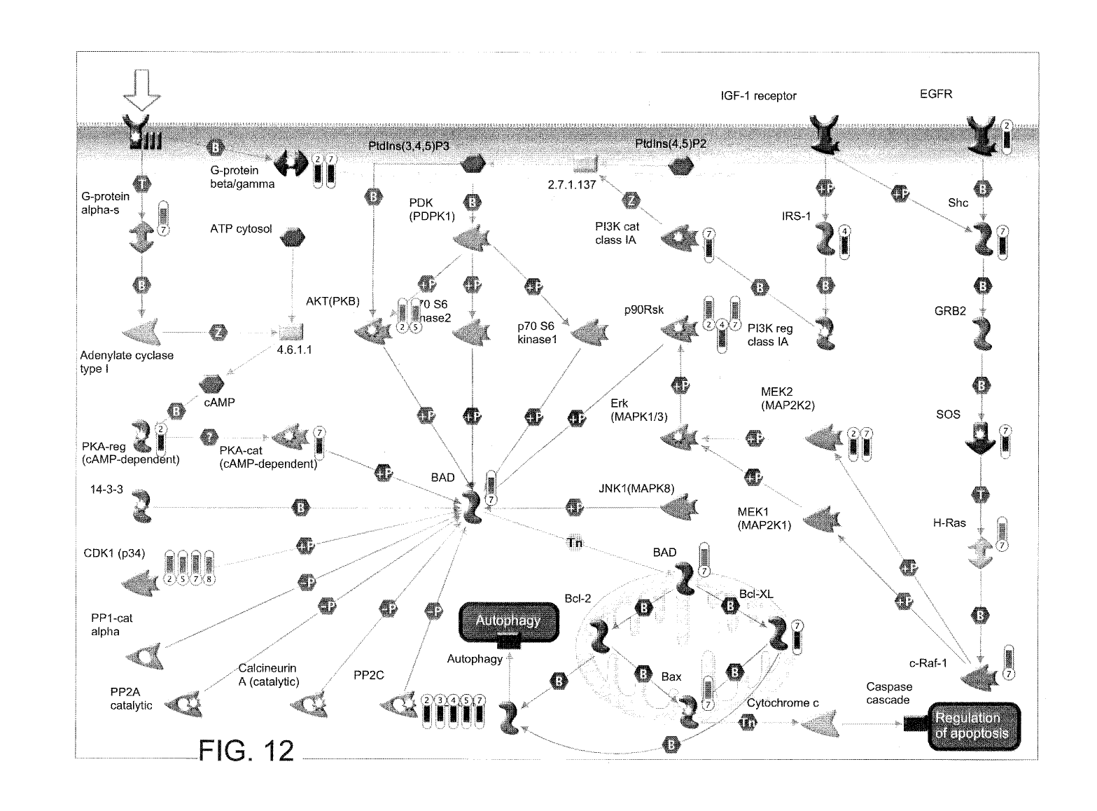

In the OVCA cell lines subjected to serial cisplatin treatment, expression of 3,111 unique probe sets, representing 2,434 unique genes, correlated across dose levels with cisplatin-resistance measured by EC50 (Pearson correlation coefficients >0.85, absolute value). GeneGO MetaCore™ analysis identified representation of the “BAD phosphorylation, apoptosis and survival” pathway to be associated with development of in vitro cisplatin-resistance (P<0.001) (FIG. 12). Statistical significance was derived from the number of genes imputed into the analysis software, the number of imputed genes present in a specific pathway, and the actual number of genes in that pathway. Thus, the P value represents the probability that mapping a set of genes to a particular pathway occurs by chance. BAD-pathway genes found to be associated with the evolution of in vitro cisplatin-resistance included BAD, Bax, BcL-XL, PP2C/PPM1A, AKT, EGFR, IRS-1, She, H-Ras, CDK1, G-protein alpha-s, G-protein beta/gamma, PI3K cat class 1A, c-Raf-1, p90Rsk, MEK2 (MAP2K2), PKA-cat, and PKA-reg (FIG. 12).

B. Genes Associated with Patient OVCA Platinum Response:

In 142 OVCA patient samples, 397 probe sets, representing 347 unique genes, were identified as differentially expressed (P<0.01) between CR versus IR primary OVCAs (Supplementary Table S2). Pathway analysis of these 347 unique genes demonstrated representation of the “BAD-phosphorylation, apoptosis and survival” pathway approaching statistical significance (P<0.08).

C. BAD Phosphorylation Status is Associated with In Vitro and In Vivo Chemoresistance:

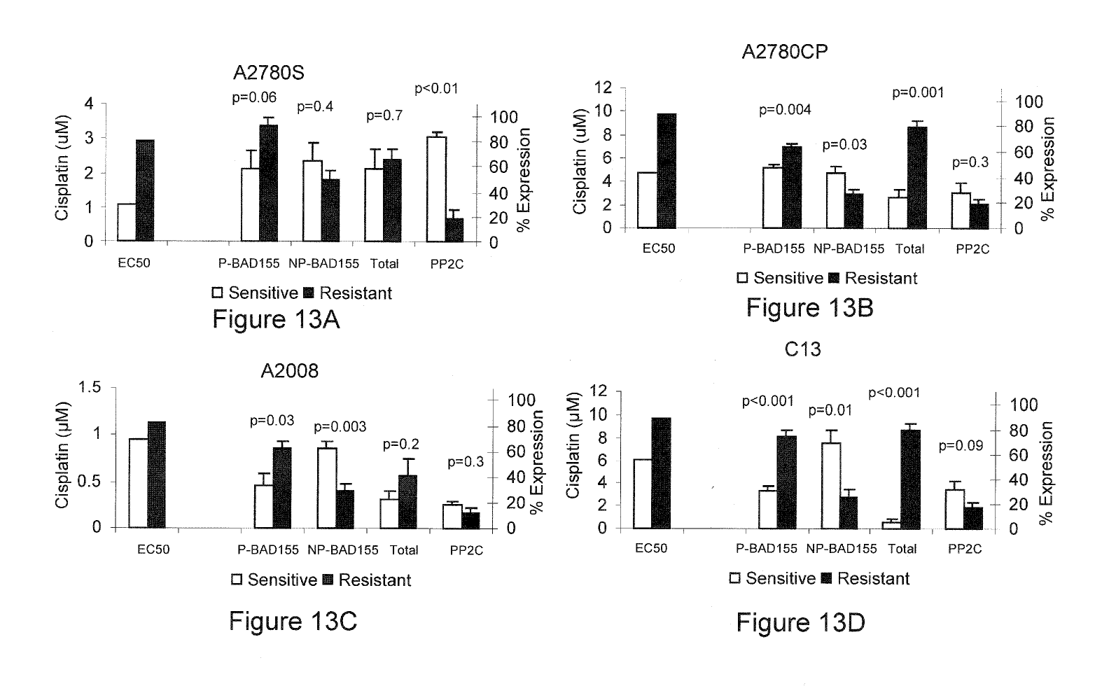

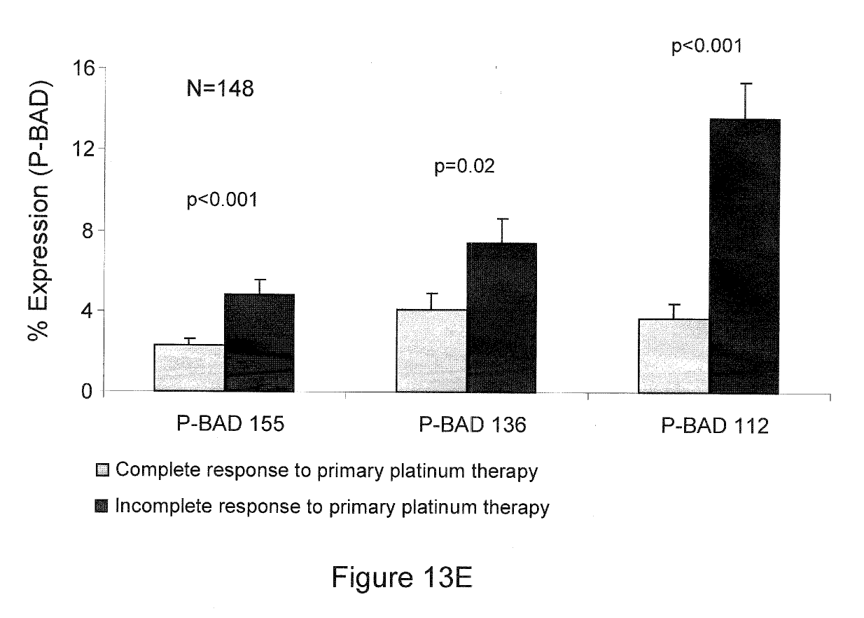

Many of the BAD-pathway genes found to be associated with evolution of in vitro cisplatin-resistance are known to influence BAD phosphorylation (FIG. 12). The inventors therefore tested the hypothesis that BAD-phosphorylation status is associated with OVCA cisplatin-resistance. Protein levels of total BAD, phosphorylated BAD (pBAD; serine-112, -136, and -155), the non-phosphorylated form of BAD (serine-155), and the BAD phosphatase PP2C/PPM1A were evaluated by immunofluorescence in 1) OVCA cell lines subjected to serial cisplatin treatment (A2780S, A2780CP, A2008, C13) and 2) 148 primary OVCA samples. OVCA cell lines subjected to in vitro cisplatin-treatment/expansion cycles demonstrated higher cisplatin EC50 values and corresponding higher levels of both pBAD (serine-55) and total BAD than those cells prior to serial cisplatin treatment (FIGS. 13A-D). In contrast, protein levels of the non-phosphorylated form of BAD (serine-155) and PP2C/PPM1A were expressed at lower levels in serially cisplatin-treated cells. Analysis of 148 patient primary OVCAs consistently revealed higher levels of pBAD (serine-112, -136, and -155) in platinum-resistant (IR) than in platinum-sensitive (CR) samples (P<0.001, P=0.02, P<0.001, respectively) (FIG. 13E).

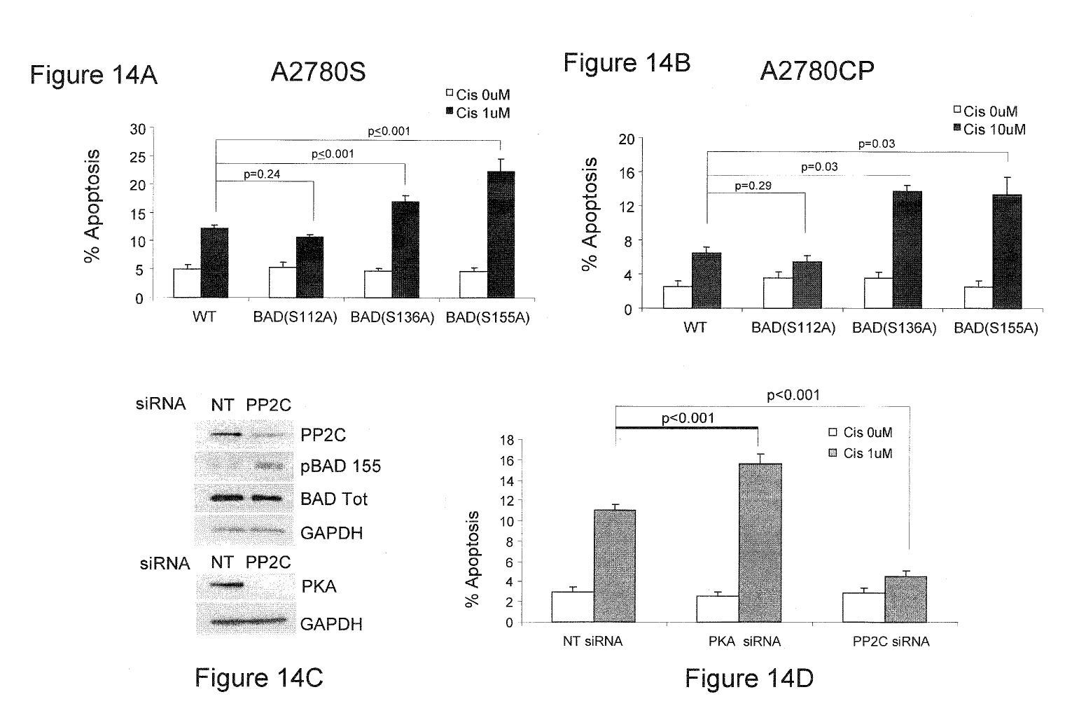

Direct and indirect modulation of BAD phosphorylation status influenced cisplatin sensitivity in OVCA cell lines. Over-representation of non-phosphorylated BAD by transfection of A2780S and A2780CP cells with vectors containing serine (S) to alanine (A) mutations (BAD[S136A], BAD[S155A]) in BAD (site mutations that prevent phosphorylation of the BAD protein) resulted in increased cisplatin-induced apoptosis compared to cells transfected with wild-type BAD (FIGS. 14A and 14B). In contrast, cells transfected with BAD[112A] had no effect on cisplatin sensitivity (FIGS. 14A and 14B).

The role of pBAD (serine-155) in cisplatin sensitivity was further evaluated in A2780S cells by depletion of PKA and PP2C/PPM1A using siRNA. Depletion of PKA and PP2C/PPM1A resulted in reduced target protein expression (FIG. 14C). Depletion of PKA decreased pBAD levels and increased cisplatin-induced apoptosis compared to cells transfected with a non-targeting control siRNA. In contrast, cells depleted of PP2C/PPM1A demonstrated increased pBAD levels and decreased cisplatin-induced apoptosis (FIGS. 14C and 14D).

D. Analysis of the BAD Pathway in the 60 NCI Cancer Cell Line Panel:

To explore the influence of the BAD pathway on the chemosensitivity of several cancer cell types, the inventors evaluated genomic and chemosensitivity data for the 60 NCI cell line panel. Analyzing all cell types together, GeneGo/MetaCore™ identified representation of the BAD pathway in genes differentially expressed in cells sensitive versus those resistant to carboplatin (P=0.001), paclitaxel (P=0.015), gemcitabine (P=0.001), and cyclophosphamide (P=0.001) but not to docetaxel, doxorubicin, topotecan, or cisplatin (Table S3 in Supplementary Appendix). Similarly, this NCI dataset was analyzed by cancer cell type for representation of the BAD pathway associated with sensitivity to individual drugs. Thus, the inventors found that the BAD pathway was associated with chemosensitivity of OVCA cells to carboplatin (P=0.01), breast cancer cells to carboplatin (P=0.04) and topotecan (P=0.03), leukemia cells to carboplatin and gemcitabine (P=0.03), melanoma cells to paclitaxel (P=0.02), non-small cell lung cancer cells to cyclophosphamide (P=0.02), and colon cancer cells to paclitaxel and docetaxel (P=0.03) (Table S4 in Supplementary Appendix).

E. A 47-Gene BAD-Pathway mRNA Gene Signature is Associated with Human Cancer Clinical Outcome:

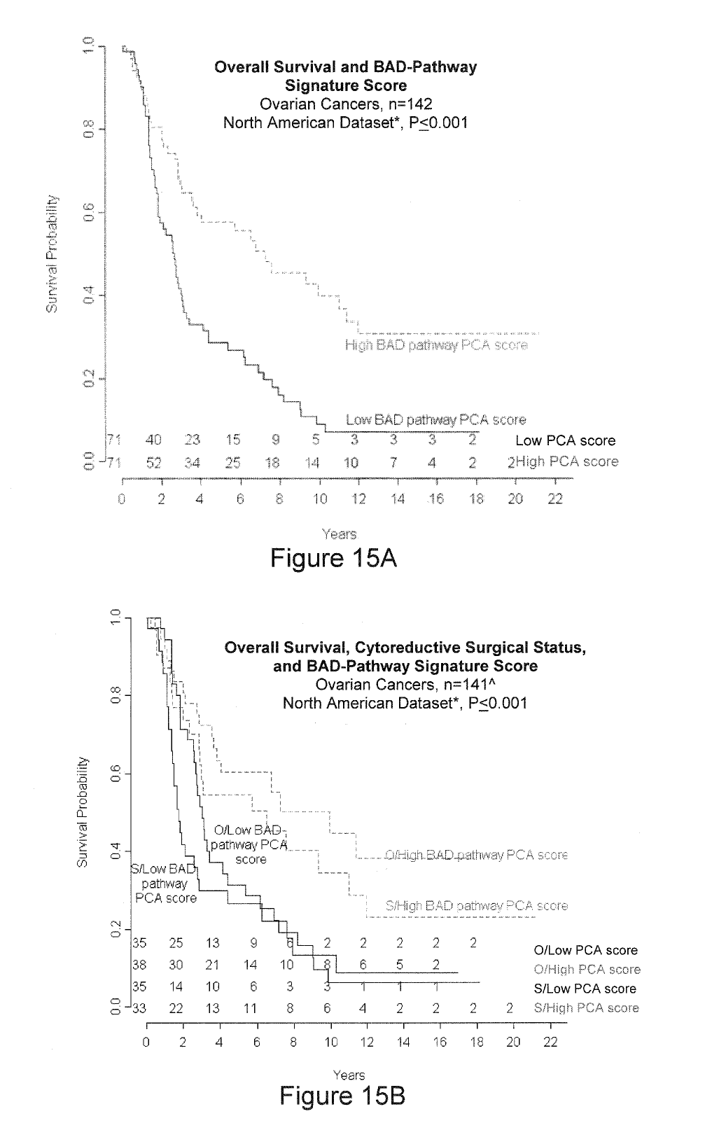

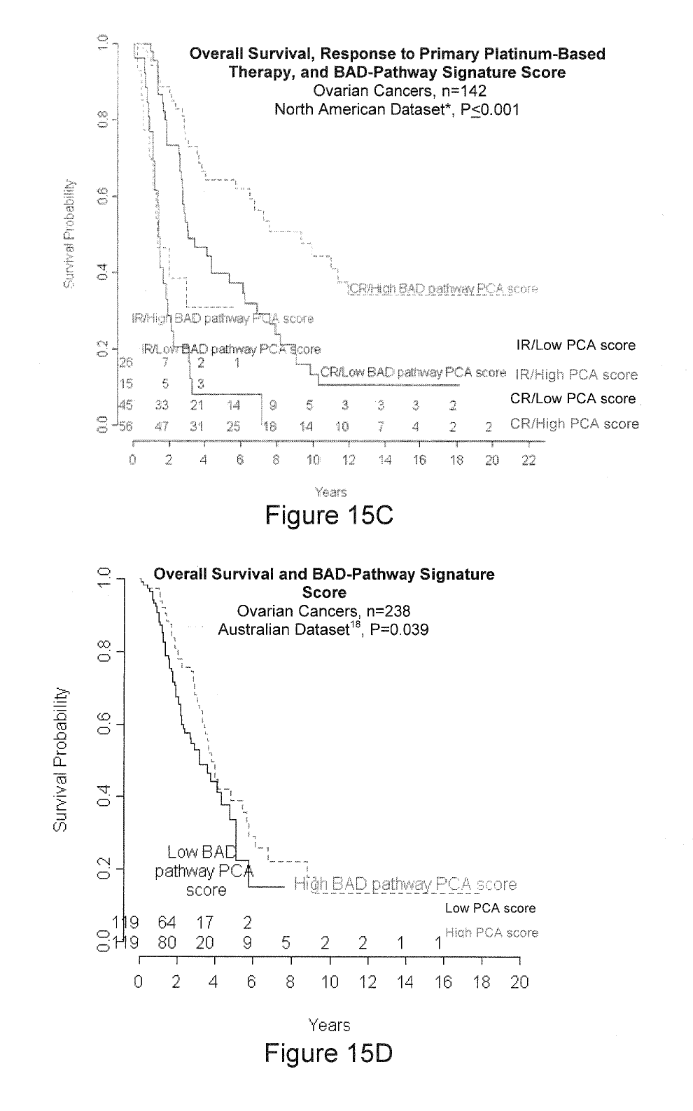

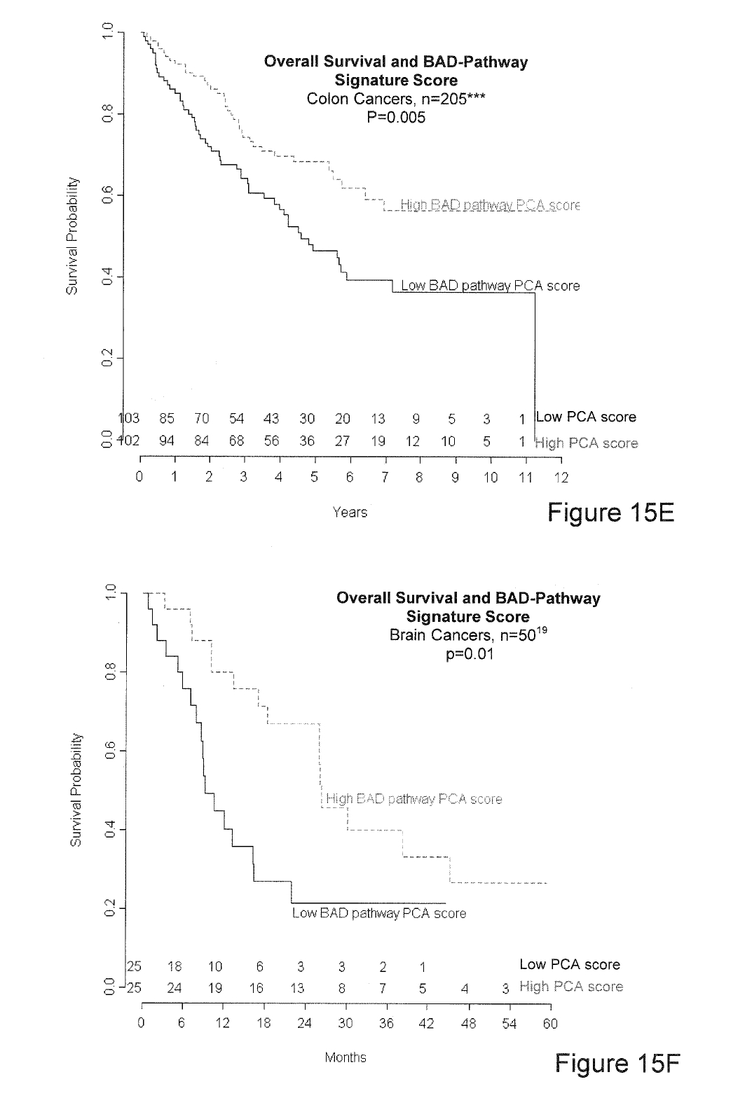

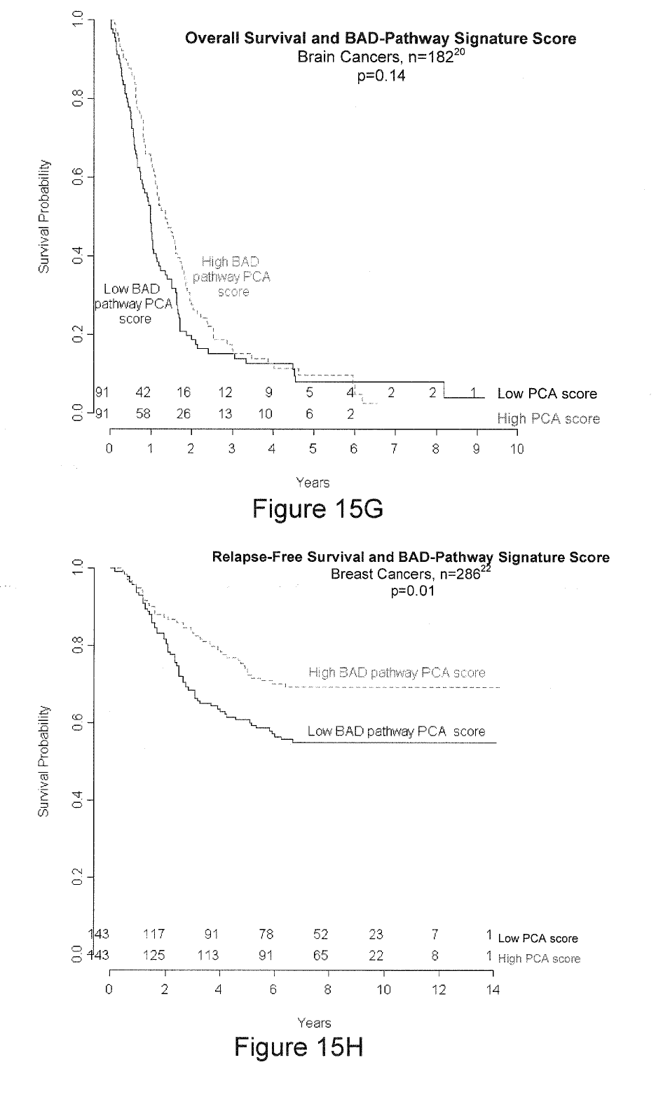

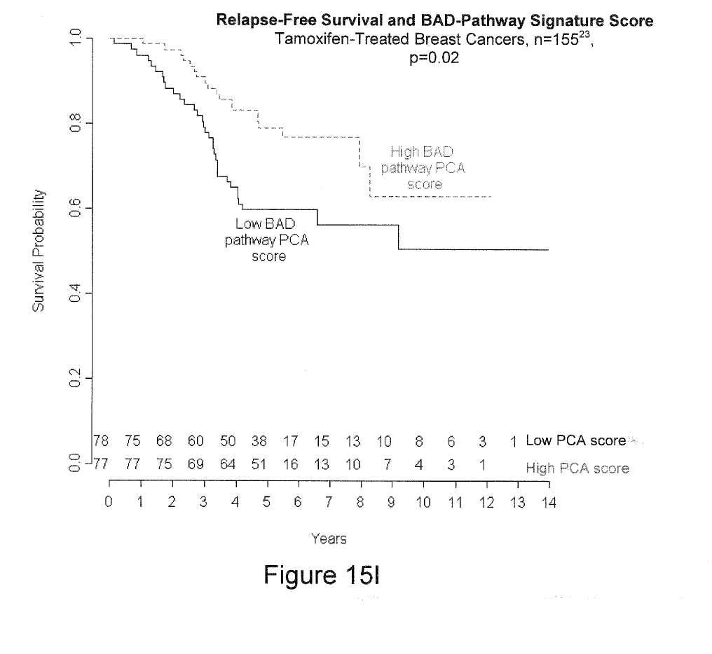

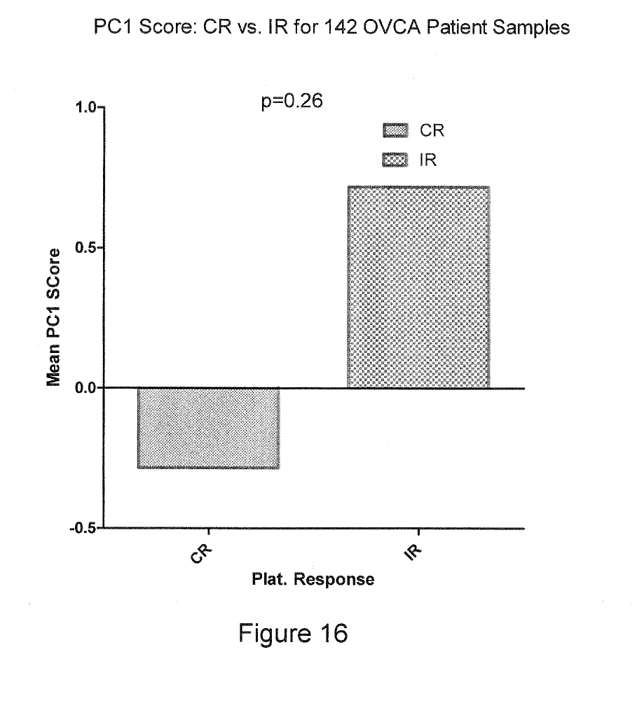

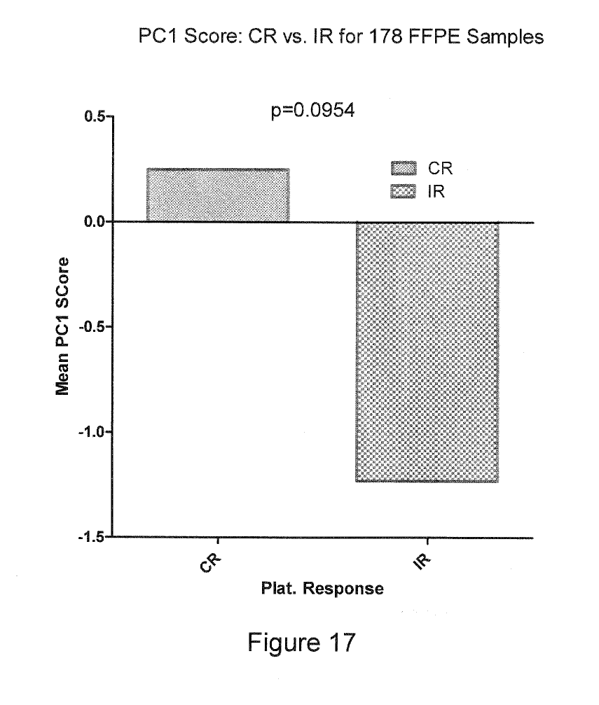

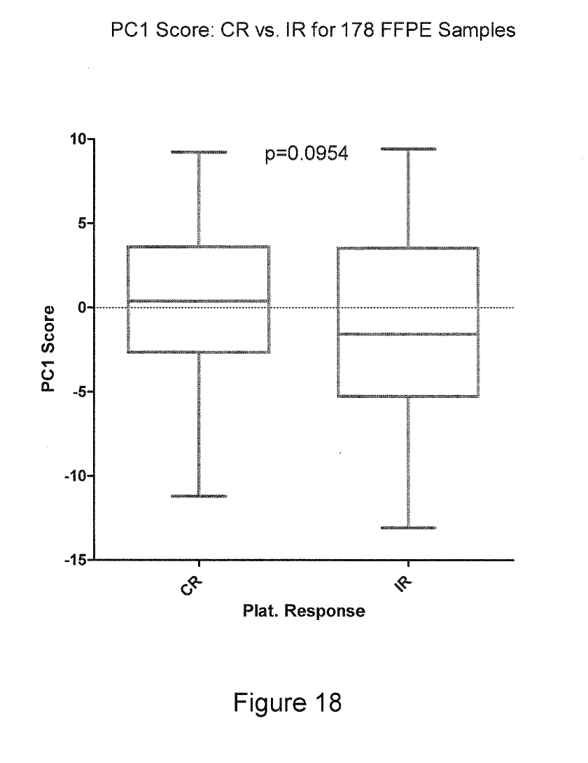

Based on the above data, the inventors designed and evaluated a 47-gene BAD-pathway mRNA signature (Table S5 in Supplementary Appendix). A BAD-pathway signature score was calculated based on the first principal component to represent the overall expression level for the BAD pathway and was evaluated in 7 external clinical-genomic datasets representing multiple different tumor types from 1,258 patient samples. The BAD-pathway score was associated with overall survival from OVCA (2 datasets: n=142, P=0.001, FIG. 15A; n=238, P=0.04, FIG. 15D), colon cancer (n=205, P=0.005, FIG. 15E), brain cancer (2 datasets: n=50, P=0.01, FIG. 15F; n=182, P=0.14, FIG. 15G), and relapse-free survival from breast cancer (2 datasets: n=286, P=0.01, FIG. 15H; n=155, P=0.02, FIG. 15I). Furthermore, the North American OVCA dataset was evaluated with regard to BAD-pathway score and surgical cytoreductive (debulking) status (optimal: <1 cm; suboptimal: >1 cm residual tumor at conclusion of surgery, P=0.001, FIG. 15B) and also response to primary platinum-based therapy (CR or IR, P=0.001, FIG. 15C). The association of high BAD-pathway score and favorable outcome was observed in patients who underwent optimal and suboptimal debulking (optimal: P=0.003, suboptimal: P=0.014). Most importantly, OVCA patients with a high BAD-pathway score who underwent suboptimal debulking had a survival that trended toward superiority compared to patients with a low BAD-pathway score who underwent optimal debulking (P=0.064). Similarly, patients who demonstrated an IR to primary platinum-based therapy but had a high BAD-pathway score had a survival equivalent to those patients who demonstrated a CR but had a low BAD-pathway score (P=0.684). When evaluated with debulking status and response to primary platinum-based therapy, grade, and age, the BAD-pathway score was an independent variable associated with survival (P=0.018).

Few clinical or biologic events impact patient outcome more than response to chemotherapy. In the current study, using a novel in vitro strategy and OVCA as a model, the inventors identified and characterized the BAD-apoptosis pathway to be influential in the response shown in a range of human cancers to a variety of chemotherapies, likely via modulation of BAD phosphorylation; the inventors also identified this pathway to be independently associated with clinical outcome for many human cancers. The inventors provide extensive validation of these findings (and the importance of the BAD pathway), with in-vitro functional studies in addition to in vivo and in silico analyses of >1,400 patient specimens and/or datasets. The inventors' findings are consistent with prior studies reporting that some of these BAD pathway signature genes, including RAF1, BAD, GNG5, PPM1B, PPM1F, GNAS, PRKAR1A, BAX, PIK3CD, and PTPN11, are associated with OVCA chemoresponse.^{24,25}

Using an in vitro model to induce OVCA cisplatin-resistance, the inventors have identified expression of BAD-apoptosis pathway genes to be associated with the evolution of cisplatin-resistance and recognized that many of these genes are kinases or phosphatases that influence the phosphorylation status of the BAD protein. Consistently, the inventors found that levels of pBAD increased with OVCA cisplatin-resistance in both the cell lines and primary patient samples that were analyzed. To validate these findings and the importance of the BAD pathway, the inventors demonstrated that in vitro manipulation of BAD-phosphorylation levels (by siRNA depletion of a BAD kinase or BAD phosphatase or by targeted mutagenesis of key BAD-phosphorylation sites) resulted in a corresponding change in cisplatin sensitivity. Further validation of the in vitro and in vivo findings is provided by in silico analysis of genomic and chemosensitivity data from 60 cancer cell lines representing 9 tumor types and 8 different chemotherapeutics: a similar representation was shown of BAD-pathway genes associated with chemosensitivity, suggesting that the pathway may not only influence OVCA cell sensitivity to platinum but may also influence many other cancer cell types to a range of different chemotherapeutic agents. To support and further explore the clinical relevance of these findings, the inventors developed a 47-gene BAD-pathway signature and evaluated it in 7 discrete clinical-genomic datasets obtained from >1,200 patients worldwide and demonstrated that a high BAD-pathway signature score is associated with favorable disease-free and/or survival in all tumor types examined. Importantly, analysis of OVCA genomic data from 142 patients with advanced-stage disease suggested that the influence of the BAD pathway on overall survival may be more important than the volume of residual disease at the completion of primary surgery, traditionally one of the most important clinical determinants of outcome for patients with OVCA. These findings may be used to improve clinical treatment of patients with this disease.

In addition to characterizing a mechanism by which human cancers develop resistance to chemotherapy, the inventors have identified a pathway that has significant clinical relevance as a potential biomarker of therapeutic response, overall patient survival, and also as a promising therapeutic target.

### REFERENCES

1. Baker W. Salvage therapy for recurrent epithelial ovarian cancer. Hematol Oncol Clin North Am 2003; 17:977-988.
2. Hansen H H, Eisenhauer E A, Hansen M, et al. New cytostatic drugs in ovarian cancer. Ann Oncol 1993; 4 Suppl 4:63-70.
3. Herrin V E, Thigpen J T. Chemotherapy for ovarian cancer: current concepts. Semin Surg Oncol 1999; 17:181-188.
4. Godwin A K, Meister A, O'Dwyer P J, Huang C S, Hamilton T C, Anderson M E. High resistance to cisplatin in human ovarian cancer cell lines is associated with marked increase of glutathione synthesis. Proc Natl. Acad Sci USA 1992; 89:3070-3074.
5. Johnson S W, Laub P B, Beesley J S, Ozols R F, Hamilton T C. Increased platinum-DNA damage tolerance is associated with cisplatin resistance and cross-resistance to various chemotherapeutic agents in unrelated human ovarian cancer cell lines. Cancer Res 1997; 57:850-856.
6. Johnson S W, Swiggard P A, Handel L M, et al. Relationship between platinum-DNA adduct formation and removal and cisplatin cytotoxicity in cisplatin-sensitive and -resistant human ovarian cancer cells. Cancer Res 1994; 54:5911-5916.
7. Benedetti V, Perego P, Luca Beretta G, et al. Modulation of survival pathways in ovarian carcinoma cell lines resistant to platinum compounds. Mol Cancer Ther 2008; 7:679-687.
8. Dressman H K, Berchuck A, Chan G, et al. An integrated genomic-based approach to individualized treatment of patients with advanced-stage ovarian cancer. J Clin Oncol 2007; 25:517-525.
9. Jazaeri A A, Awtrey C S, Chandramouli G V, et al. Gene expression profiles associated with response to chemotherapy in epithelial ovarian cancers. Clin Cancer Res 2005; 11:6300-6310.
10. Boren T, Xiong Y, Hakam A, et al. MicroRNAs and their target messenger RNAs associated with ovarian cancer response to chemotherapy. Gynecol Oncol 2009; 113:249-255.
11. Bild A H, Yao G, Chang J T, et al. Oncogenic pathway signatures in human cancers as a guide to targeted therapies. Nature 2006; 439:353-357.
12. Irizarry R A, Hobbs B, Collin F, et al. Exploration, normalization, and summaries of high density oligonucleotide array probe level data. Biostatistics 2003; 4:249-264.
13. Bolstad B M, Irizarry R A, Astrand M, Speed T P. A comparison of normalization methods for high density oligonucleotide array data based on variance and bias. Bioinformatics 2003; 19:185-193.
14. Chen N, Kamath S, Newcomb J, et al. Trophic factor induction of human umbilical cord blood cells in vitro and in vivo. J Neural Eng 2007; 4:130-145.
15. Marchion D C, Bicaku E, Turner J G, Daud Al, Sullivan D M, Munster P N. Synergistic interaction between histone deacetylase and topoisomerase II inhibitors is mediated through topoisomerase Ilbeta. Clin Cancer Res 2005; 11:8467-8475.
16. Efron B, Tibshirani R. Empirical Bayes methods and false discovery rates for microarrays. Genet Epidemiol 2002; 23:70-86.
17. Tusher V G, Tibshirani R, Chu G. Significance analysis of microarrays applied to the ionizing radiation response. Proc Natl Acad Sci USA 2001; 98:5116-5121.
18. Tothill R W, Tinker A V, George J, et al. Novel molecular subtypes of serous and endometrioid ovarian cancer linked to clinical outcome. Clin Cancer Res 2008; 14:5198-5208.
19. Nutt C L, Mani D R, Betensky R A, et al. Gene expression-based classification of malignant gliomas correlates better with survival than histological classification. Cancer Res 2003; 63:1602-1607.
20. Lee Y, Scheck A C, Cloughesy T F, et al. Gene expression analysis of glioblastomas identifies the major molecular basis for the prognostic benefit of younger age. BMC Med Genomics 2008; 1:52.
21. Carroll J S, Meyer C A, Song J, et al. Genome-wide analysis of estrogen receptor binding sites. Nat Genet 2006; 38:1289-1297.
22, Wang Y, Klijn J G, Zhang Y, et al. Gene-expression profiles to predict distant metastasis of lymph-node-negative primary breast cancer. Lancet 2005; 365:671-679.
23. Chanrion M, Negre V, Fontaine H, et al. A gene expression signature that can predict the recurrence of tamoxifen-treated primary breast cancer. Clin Cancer Res 2008; 14:1744-1752.
24. Klumpp S, Selke D, Krieglstein J. Protein phosphatase type 2C dephosphorylates BAD. Neurochem Int 2003; 42:555-560.
25. Yang L, Omori K, Suzukawa J, Inagaki C. Calcineurin-mediated BAD Ser155 dephosphorylation in ammonia-induced apoptosis of cultured rat hippocampal neurons. Neurosci Lett 2004; 357:73-75.
26. Danial N N, Korsmeyer S J. Cell death: critical control points. Cell 2004; 116:205-219.
27. Dejean L M, Martinez-Caballero S, Guo L, et al. Oligomeric Bax is a component of the putative cytochrome c release channel MAC, mitochondrial apoptosis-induced channel. Mol Biol Cell 2005; 16:2424-2432.
28. Desagher S, Osen-Sand A, Nichols A, et al. Bid-induced conformational change of Bax is responsible for mitochondrial cytochrome c release during apoptosis. J Cell Biol 1999; 144:891-901.
29. Kuwana T, Mackey M R, Perkins G, et al. Bid, Bax, and lipids cooperate to form supramolecular openings in the outer mitochondrial membrane. Cell 2002; 111:331-342.
30. Yang E, Zha J, Jockel J, Boise L H, Thompson C B, Korsmeyer S J. Bad, a heterodimeric partner for Bcl-XL and Bcl-2, displaces Bax and promotes cell death. Cell 1995; 80:285-291.
31. del Peso L, Gonzalez-Garcia M, Page C, Herrera R, Nunez G. Interleukin-3-induced phosphorylation of BAD through the protein kinase Akt. Science 1997; 278:687-689.
32. Lizcano J M, Mon-ice N, Cohen P. Regulation of BAD by cAMP-dependent protein kinase is mediated via phosphorylation of a novel site, Ser155. Biochem J 2000; 349:547-557.
33. Tan Y, Demeter M R, Ruan H, Comb M J. BAD Ser-155 phosphorylation regulates BAD/Bcl-XL interaction and cell survival. J Biol Chem 2000; 275:25865-25869.
34. Zhou X M, Liu Y, Payne G, Lutz R J, Chittenden T. Growth factors inactivate the cell death promoter BAD by phosphorylation of its BH3 domain on Ser155. J Biol Chem 2000; 275:25046-25051.

1. A method for preparing a gene expression profile indicative of cancer prognosis, chemo-resistance or chemo-sensitivity, comprising determining the level of expression for a plurality of genes of the BCL2 antagonist of cell death (BAD) pathway in a biological sample, thereby preparing the gene expression profile.

2. The method of claim 1, wherein the prognosis is with respect to at least one factor selected from the group consisting of overall survival, disease- or relapse-free survival, relapse, and disease progression.

3-4. (canceled)

5. The method of claim 1, wherein the plurality of genes of the BAD pathway comprises a plurality of genes listed in Table 1.

6. (canceled)

7. The method of claim 5, wherein the plurality of genes comprises or the following 53 BAD pathway genes:
MAP2K2, RAF1, HRAS, SCH1, EGFR, IRS1, PIK3CA, PIK3CB, PIK3CD, RPS6KA1, RPS6KA2, RPS6KA3, BAD, BCL2L1, BAX, AKT1, AKT2, AKT3, GNB1, GNB2, GNB3, GNB4, GNB5, GNG10, GNG11, GNG12, GNG13, GNG2, GNG3, GNG4, GNG5, GNG7, GNG8, GNGT1, GNGT2, PPM1A, PPM1B, PPM1D, PPM1F, PPM1G, PPM1L, PPM2C, PPTC7, PTPN11, GNAS, PRKAR1A, PRKAR1B, PRKAR2A, PRKAR2B, CDC2, PRKACA, PRKACB, and PRKACG.

8. (canceled)

9. The method of claim 5, wherein the plurality of genes comprises the following 47 BAD pathway genes:
MAP2K2, RAF1, HRAS, SCH1, EGFR, IRS1, PIK3CA, PIK3CB, PIK3CD, RPS6KA1, RPS6KA2, RPS6KA3, BAD, BCL2L1, BAX, AKT1, AKT2, AKT3, GNB1, GNB2, GNB3, GNB5, GNG10///LOC552891, GNG11, GNG12, GNG13, GNG3, GNG4, GNG5, GNG7, GNGT1, PPM1A, PPM1B, PPM1D, PPM1F, PPM1G, PPM2C, PTPN11, GNAS, PRKAR1A, PRKAR1B, PRKAR2A, PRKAR2B, CDC2, PRKACA, PRKACB, and PRKACG.

10. The method of claim 1, wherein the gene expression profile is expressed as a pathway score representative of the overall expression level for the plurality of genes.

11. The method of claim 10, wherein the pathway score is defined as:
Σwᵢxᵢ, a weighted average expression among the plurality of BAD pathway genes, where xᵢ represents gene i expression level, wᵢ is the corresponding weight (loading coefficient) with Σwᵢ²=1, and the wᵢ values maximize the variance of Σwᵢxᵢ.

12-20. (canceled)

21. A method for assessing the chemo-sensitivity or chemo-resistance of a cancer, or the prognosis of cancer in a subject, comprising determining the level of expression of a plurality of Genes of the BCL2 antagonist of cell death (BAD) pathway in a biological sample; and comparing the determined level of expression in the sample to a reference BAD pathway gene expression level.

22. The method of claim 21, wherein the sample gene expression profile and the reference gene expression are each expressed as a pathway score representative of the overall expression level for the plurality of genes.

23. The method of claim 22, wherein the pathway score is defined as:
Σwᵢxᵢ, a weighted average expression among the plurality of BAD pathway genes, where x, represents gene i expression level, wᵢ is the corresponding weight (loading coefficient) with Σwᵢ²=1, and the wᵢ values maximize the variance of Σwᵢxᵢ.

24-25. (canceled)

26. The method of claim 21, wherein the prognosis is with respect to at least one factor selected from the group consisting of overall survival, disease- or relapse-free survival, relapse, and disease progression.

27-28. (canceled)

29. The method of claim 21, wherein the plurality of genes of the BAD pathway comprises a plurality of genes listed in Table 1.

30. (canceled)

31. The method of claim 21, wherein the plurality of genes comprises the following 53 BAD pathway genes:
MAP2K2, RAF1, HRAS, SCH1, EGFR, IRS1, PIK3CA, PIK3CB, PIK3CD, RPS6KA1, RPS6KA2, RPS6KA3, BAD, BCL2L1, BAX, AKT1, AKT2, AKT3, GNB1, GNB2, GNB3, GNB4, GNB5, GNG10, GNG11, GNG12, GNG13, GNG2, GNG3, GNG4, GNG5, GNG7, GNG8, GNGT1, GNGT2, PPM1A, PPM1B, PPM1D, PPM1F, PPM1G, PPM1L, PPM2C, PPTC7, PTPN11, GNAS, PRKAR1A, PRKAR1B, PRKAR2A, PRKAR2B, CDC2, PRKACA, PRKACB, and PRKACG.

32. (canceled)

33. The method of claim 21, wherein the plurality of genes comprises the following 47 BAD pathway genes:
MAP2K2, RAF1, HRAS, SCH1, EGFR, IRS1, PIK3CA, PIK3CB, PIK3CD, RPS6KA1, RPS6KA2, RPS6KA3, BAD, BCL2L1, BAX, AKT1, AKT2, AKT3, GNB1, GNB2, GNB3, GNB5, GNG10///LOC552891, GNG11, GNG12, GNG13, GNG3, GNG4, GNG5, GNG7, GNGT1, PPM1A, PPM1B, PPM1D, PPM1F, PPM1G, PPM2C, PTPN11, GNAS, PRKAR1A, PRKAR1B, PRKAR2A, PRKAR2B, CDC2, PRKACA, PRKACB, and PRKACG.

34-47. (canceled)

48. A method for treating cancer in a subject, comprising:
(a) administering an agent that targets the BCL2 antagonist of cell death (BAD) pathway; or
(b) assessing the prognosis of cancer in the subject, comprising comparing the level of expression of a plurality of genes of the BCL2 antagonist of cell death (BAD) pathway in a sample from the subject to a reference BAD pathway gene expression level; and administering an agent that targets the BAD pathway to the subject if the subject is assessed to have a poor or undesirable prognosis; or
(c) administering a chemotherapeutic agent to the subject, wherein the cancer is predetermined to be chemo-sensitive based on the level of expression of a plurality of genes of the BCL2 antagonist of cell death (BAD) pathway; or
(d) assessing the chemo-sensitivity or chemo-resistance of the cancer, comprising comparing the level of expression of a plurality of genes of the BCL2 antagonist of cell death (BAD) pathway in a sample of the cancer to a reference BAD pathway gene expression level; and (b) administering a chemotherapeutic agent to the subject if the cancer is determined to be chemo-sensitive (sensitive to the chemotherapeutic agent) based on said assessing.

49. The method of claim 48, comprising (a), wherein the subject is predetermined to have a poor cancer prognosis based on the level of expression of a plurality of genes of the BAD pathway.

50-56. (canceled)

57. A composition of matter comprising:
(a) a computer system for performing:
(i) a method for preparing a gene expression profile indicative of cancer prognosis, chemo-resistance or chemo-sensitivity, comprising: determining the level of expression for a plurality of genes of the BCL2 antagonist of cell death (BAD) pathway in a biological sample, thereby preparing the gene expression profile; or
(ii) a method for assessing the chemo-sensitivity or chemo-resistance of a cancer, or the prognosis of cancer in a subject, comprising determining the level of expression of a plurality of genes of the BCL2 antagonist of cell death (BAD) pathway in a biological sample; and comparing the determined level of expression in the sample to a reference BAD pathway gene expression level; or
(iii) a method for treating cancer in a subject, comprising administering an agent that targets the BCL2 antagonist of cell death (BAD) pathway; or
(iv) a method for treating cancer in a subject, comprising: (a) assessing the prognosis of cancer in the subject, comprising comparing the level of expression of a plurality of genes of the BCL2 antagonist of cell death (BAD) pathway in a sample from the subject to a reference BAD pathway gene expression level; and (b) administering an agent that targets the BAD pathway to the subject if the subject is assessed to have a poor or undesirable prognosis; or
(v) A method for treating cancer in a subject, comprising: (a) assessing the prognosis of cancer in the subject, comprising comparing the level of expression of a plurality of genes of the BCL2 antagonist of cell death (BAD) pathway in a sample from the subject to a reference BAD pathway gene expression level; and (b) administering an agent that targets the BAD pathway to the subject if the subject is assessed to have a poor or undesirable prognosis; or
(vi) a method for treating cancer in a subject, comprising: (a) assessing the chemo-sensitivity or chemo-resistance of the cancer, comprising comparing the level of expression of a plurality of genes of the BCL2 antagonist of cell death (BAD) pathway in a sample of the cancer to a reference BAD pathway gene expression level; and (b) administering a chemotherapeutic agent to the subject if the cancer is determined to be chemo-sensitive (sensitive to the chemotherapeutic agent) based on the assessment of (a); or
(b) probe array or probe set for performing the method of any one of (a)(i)-(a)(vi), comprising a plurality of probes that hybridize to a plurality of nucleic acids of the BCL2 antagonist of cell death (BAD) pathway; or
(c) a kit for performing the method of any one of (a)(i)-(a)(vi), comprising the probe array or probe set of (b).
